(19) United States
(12) Patent Application Publication
(10) Pub. No.: US 2024/0197872 A1
(43) Pub. Date: Jun. 20, 2024

(54) NOVEL COMPOSITIONS ENRICHED IN GAMMA DELTA T CELLS, METHODS OF PREPARATION, AND USES THEREOF

(71) Applicant: Acepodia Biotechnologies Ltd., New Taipei City (TW)

(72) Inventors: CHING-WEN HSIAO, Castro Valley, CA (US); ZIH-FEI CHENG, New Taipei City (TW); TAI-SHENG WU, New Taipei City (TW); HAO-KANG LI, New Taipei City (TW); HSIU-PING YANG, New Taipei City (TW); CHIA-YUN LEE, New Taipei City (TW); SAI-WEN TANG, San Mateo, CA (US); YI-HUNG OU, New Taipei City (TW); YAN-LIANG LIN, New Taipei City (TW); SHIH-CHIA HSIAO, New Taipei City (TW)

(73) Assignee: Acepodia Biotechnologies Ltd., New Taipei City (TW)

(21) Appl. No.: 18/286,286

(22) PCT Filed: Apr. 14, 2022
(86) PCT No.: PCT/US2022/024775

§ 371 Date: Oct. 10, 2023

Related U.S. Application Data
(60) Provisional application No. 63/175,689, filed on Apr. 16, 2021.
(60) Provisional application No. 63/253,323, filed on Oct. 7, 2021.

(51) Int. Cl. A61K 39/00; A61P 35/00; C12N 5/0783

(52) U.S. Cl. CPC A61K 39/4611; A61K 39/4633; A61K 39/464406; A61K 39/464424; A61P 35/00; C12N 5/0636; C12N 2501/2302; C12N 2501/998; C12N 2502/115

(57) ABSTRACT

Provided herein are novel compositions enriched in gdT cells with high therapeutic potential. Methods to produce such compositions and methods of uses thereof in adoptive immunotherapies are also provided.

This application claims priority to U.S. Provisional Application No. 63/175,689, filed Apr. 16, 2021, and U.S. Provisional Application No. 63/253,323, filed Oct. 7, 2021, which are entirely incorporated herein by reference.

### 1. FIELD

The present invention relates to molecular biology, cell biology, and immunology. Provided herein are novel compositions enriched in gamma delta T (gdT) cells with NK-like properties, methods of preparation thereof, and methods of uses thereof.

### 2. BACKGROUND

Possessing both innate and adaptive-like properties, gdT cells have broad antigen specificity and NK-like cytotoxicity. Also, gdT cells can infiltrate into different tumors and kill a wide range of tumor cells. As such, many approaches to use gdT cells in immunotherapies, such as cancer immunotherapies, have been attempted, but met with limited success, largely because the methods to selectively and efficiently expand gdT cells with therapeutic potential are still lacking. Accordingly, there is an unmet need for methods of obtaining cell populations enriched in gdT cells with therapeutic potential. The present disclosures address this need and provide related advantages.

### 3. SUMMARY

Provided herein are methods of manufacturing a cell population enriched in gdT cells, comprising culturing a source cell population comprising gdT cells in a medium supplemented with (i) a phosphoantigen, (ii) a cytokine, and (iii) human platelet lysate (“HPL”).

In some embodiments of the methods provided herein, the cell population is not contacted with a feeder cell or tumor cell during the culture. In some embodiments, the methods provided herein does not include positively selecting for gdT cells.

In some embodiments of the methods provided herein, the cell population is cultured for 3 to 40 days, 4 to 40 days, 5 to 40 days, 6 to 40 days, 7 to 40 days, 10 to 40 days, 10 to 30 days, 6 to 20 days, 12 to 20 days, or 14 to 18 days.

In some embodiments, the methods provided herein further comprise depleting alpha beta T (abT) cells. In some embodiments, the abT cells are depleted around the half-time of the culture. In some embodiments, the cells are cultured for 14 to 18 days and the abT cells are depleted between Day 4 and Day 10.

In some embodiments of the methods provided herein, the cytokine is replenished during the culture. In some embodiments, the cytokine is replenished once per week, twice per week, three times per week, every other day, or daily.

In some embodiments of the methods provided herein, the cytokine is interleukin-2 (IL-2), interleukin-4 (IL-4), interleukin-6 (IL-6), interleukin-7 (IL-7), interleukin-8 (IL-8), interleukin-9 (IL-9), interleukin-12 (IL-12), interleukin-15 (IL-15), interleukin-18 (IL-18), interleukin-21 (IL-21), interleukin-33 (IL-33), or any combination thereof. In some embodiments, the cytokine is IL-2.

In some embodiments of the methods provided herein, the cytokine is supplemented at a concentration of 200-3000 IU/mL.

In some embodiments of the methods provided herein, the phosphoantigen is not replenished during the culture.

In some embodiments of the methods provided herein, the phosphoantigen is a bisphosphonate selected from the group consisting of clodronate, etidronate, alendronate, pamidronate, zoledronate (zoledronic acid), neridronate, ibandronate, and pamidronate. In some embodiments, the phosphoantigen is zoledronate. In some embodiments of the methods provided herein, the phosphoantigen is selected from the group consisting of bromohydrin pyrophosphate (BrHPP), 4-hydroxy-but-2-enyl pyrophosphate (HMBPP), isopentenyl pyrophosphate (IPP), and dimethylallyl pyrophosphate (DMAPP).

In some embodiments of the methods provided herein, the phosphoantigen is supplemented at a concentration of 0.1-20 μM.

In some embodiments of the methods provided herein, the HPL is supplemented at a concentration of 1-20 vol %.

In some embodiments of the methods provided herein, the medium comprises glucose at a concentration of 600-5000 mg/L. In some embodiments, the medium is a serum-free medium.

In some embodiments of the methods provided herein, the cell population is cultured in a device containing an air-permeable surface. In some embodiments, the device is a G-Rex device.

In some embodiments of the methods provided herein, the source cell population comprises peripheral blood mononuclear cells (PBMCs), bone marrow, umbilical cord blood, or a combination thereof. In some embodiments, the source cell population comprises PBMCs. In some embodiments, the methods provided herein further comprise obtaining the PBMCs from peripheral blood.

In some embodiments of the methods provided herein, the gdT cells in the source cell population are expanded for at least 1,000 fold during the culture. In some embodiments, at least 75% of the resulting cell population are gdT cells.

In some embodiments, the methods provided herein further comprise adding a targeting moiety to the surface of the cells in the resulting cell population. In some embodiments, the targeting moiety is complexed to the cell surface via the interaction between a first linker conjugated to the targeting moiety and a second linker conjugated to the cell surface. In some embodiments, the targeting moiety is exogenously expressed by the resulting cell population.

In some embodiments, the methods provided herein further comprise cryopreserving the cell population after the culture.

Provided herein are also populations of cells obtained by the methods described herein.

In some embodiments, provided herein are populations of cells comprising at least 70% gdT cells, wherein (1) the gdT cells express at least 400 DNAM-1 molecules per cell on average; (2) at least 30% of the gdT cells are CD69+; or both (1) and (2). In some embodiments, the gdT cells express at least 500, at least 1000, at least 2000, or at least 3000 DNAM-1 molecules per cell on average. In some embodiments, at least 40%, at least 50%, at least 55%, at least 60%, at least 65%, at least 70%, at least 75%, or at least 80% of the gdT cells are CD69+.

In some embodiments of the cell populations provided herein, at least 30%, at least 40%, at least 50%, at least 60%, at least 70%, or at least 80% of the gdT cells are terminally differentiated effector (TDEM) cells.

In some embodiments, the cell populations provided herein comprise at least 1×10⁶, at least 5×10⁶, at least 1×10⁷, at least 5×10⁷, at least 1×10⁸, at least 5×10⁸, at least 1×10⁹, at least 5×10⁹, at least 1×10¹⁰, at least 5×10¹⁰, or at least 1×10¹¹ gdT cells.

In some embodiments, the cell populations provided herein have not been positively selected for gdT cells.

In some embodiments, the cell populations provided herein haves been cultured for 20 days or less since the source cell population from which the cell population is derived or obtained from a single donor.

In some embodiments, gdT cells in the cell populations provided herein express (1) at least 400 CD56 molecules per cell on average; (2) at least 400 CD16 molecules per cell on average; (3) at least 400 NKG2D molecules per cell on average; (4) at least 400 CD107a molecules per cell on average; (5) at most 2800 PD-1 molecules per cell on average; (6) at least 5000 DNAM-1 molecules per cell on average; (7) at least 400 CD69 molecules per cell on average; or (8) at least 100 Granzyme B molecules per cell on average; or any combination thereof.

In some embodiments of the cell populations provided herein, at least 30% of the gdT cells are V62 T cells.

In some embodiments of the cell populations provided herein, at least 10% of the gdT cells comprise a targeting moiety complexed to the cell surface.

In some embodiments, the targeting moiety is not a nucleic acid. In some embodiments, the targeting moiety is an antibody or antigen binding unit that specifically binds to a biological marker on a target cell. In some embodiments, the biological marker is a tumor antigen.

In some embodiments, the gdT cells express a chimeric antigen receptor (CAR) or a T cell receptor (TCR) that comprises the antibody or antigen binding fragment.

In some embodiments, the targeting moiety is not produced by the gdT cells. In some embodiments, the targeting moiety is complexed to the cell surface via the interaction between a first linker conjugated to the targeting moiety and a second linker conjugated to the cell surface. In some embodiments, the first linker is a first polynucleotide, and the second linker is a second polynucleotide. In some embodiments, (1) the first polynucleotide has 4 to 500 nucleotides, (2) the second polynucleotide has 4 to 500 nucleotides, or both (1) and (2).

In some embodiments, the cell populations provided herein are cryopreserved.

In some embodiments, provided herein are pharmaceutical compositions comprising the cell populations provided herein and a pharmaceutically acceptable carrier.

In some embodiments, the cell populations provided herein or the pharmaceutical compositions provided herein can maintain its therapeutic potency after being stored at or below 0° C. for at least one week, at least two weeks, at least 1 month, at least 3 months, or at least 6 months.

Provided herein are also uses of the cell populations or the pharmaceutical compositions provided herein in an adoptive immunotherapy.

Provided herein are also uses of the cell populations or the pharmaceutical compositions provided herein in the treatment of a disease or disorder.

Provided herein are also methods of treating a disease or disorder in a subject in need thereof, comprising administering the cell populations or the pharmaceutical compositions provided herein to the subject.

In some embodiments, the disease or disorder is tumor or cancer. In some embodiments, the disease or disorder is an autoimmune disease, a neuronal disease, a hematopoietic cell-related disease, metabolic syndrome, a pathogenic disease, HIV or other viral infection, fungal infection, protozoan infection, or bacterial infection. In some embodiments, the subject is human.

### 4. BRIEF DESCRIPTION OF DRAWINGS

FIGS. 1A and 1B each provide a flow chart exemplifying the methods of preparing a population of cells enriched in gdT cells.

FIG. 2 provides the line graph presenting the cell number and glucose uptake of the cell population on different days of the culture.

FIGS. 3A-3C provide flow cytometry results analyzing the cell population prepared according to methods described herein (on Day 16). As shown, molecules stained included: TCRab, TCRvd2, CD16, CD3, and CD25 (FIG. 3A); CD38, CD56, CD69, CD107a, and NKG2D (FIG. 3B); and PD-1, NKp30, NKp44, NKp46, PI staining (FIG. 3C).

FIGS. 4A-4C provide flow cytometry results analyzing the PI−TCRVβ2+-gated populations of Day 16 resulting cell populations (16-Day V62 T cells). As shown, molecules stained included: TCRVδ2, CD18, TIGIT, NKG2D, DNAM-1 (FIG. 4A); CD36, CD69, PD-1, CD103, and CCR7 (FIG. 4B); and TNFα, INFγ, Granzyme B, and CD107a (FIG. 4C).

FIGS. 5A-5Q provide the standard curves of fluorescent dye-conjugated mouse antibodies (Quantum™ Simply Cellular® kit). FIG. 5A: anti-human CD56; FIG. 5B: anti-human CD16; FIG. 5C: anti-human NKG2D; FIG. 5D: anti-human NKp44; FIG. 5E: anti-human NKp46; FIG. 5F: anti-human IFNγ; FIG. 5G: anti-human DNAM-1; FIG. 5H: anti-human Granzyme B; FIG. 5I anti-human TIGIT; FIG. 5J: anti-human TNFα; FIG. 5K: anti-human CD18; FIG. 5L: anti-human TCRVd2; FIG. 5M: anti-human NKp30; FIG. 5N: anti-human PD1; FIG. 5O: anti-human CD69; FIG. 5P: anti-human CD107a; FIG. 5Q: anti-human CCR7.

FIG. 6 is the two-dimensional dot plot presenting the memory types of the V62 T cells isolated from the Day 16 resulting cell population (16-Day V62 T cells).

FIGS. 7A-7C provide flow cytometry results analyzing Control-gdT cells and ACE-gdT cells-CD20 (rituximab) cells. As shown, molecules stained included: TCRab, TCRvd2, CD16, CD3, and CD25 (FIG. 3A); CD38, CD56, CD69, CD107a, and NKG2D (FIG. 7B); and PD-1, NKp30, NKp44, NKp46, PI staining (FIG. 7C).

FIGS. 8A-8C provide flow cytometry results analyzing the PI−TCRVβ2+-gated populations of the Control-gdT cells and ACE-gdT cells-CD20 (rituximab) cells. As shown, molecules stained included: TCRVδ2, CD18, TIGIT, NKG2D, DNAM-1 (FIG. 8A); CD36, CD69, PD-1, CD103, and CCR7 (FIG. 8B); and TNFα, INFγ, Granzyme B, and CD107a (FIG. 8C).

FIG. 9 is the two-dimensional dot plot presenting the memory types of PI−TCRVβ2+-gated populations of Control-gdT cells and ACE-gdT cells-CD20 (rituximab) cells.

FIGS. 10A-10B provide results of the cytotoxicity assay against human ovarian cancer cell line SK-OV-3. FIG. 10A shows the results comparing the cytotoxicity of the Control-gdT cells in the presence of trastuzumab and that of trastuzumab alone. FIG. 10B shows the results comparing the cytotoxicity of the ACE-gdT cells-HER2 (trastuzumab) cells and that of Control-gdT cells.

FIGS. 11A-11C provide results of the cytotoxicity assay against three cancer cell lines: CD20-positive human lymphoma cell line Raji cells (FIG. 11A); CD20-positive human lymphoma cell line Daudi (FIG. 11B); and human lymphoma cell line K562 (FIG. 11C). Each panel provides the results comparing the cytotoxicity of the ACE-gdT cells-CD20 (rituximab) cells and that of Control-gdT cells.

FIGS. 12A-12C provide results of the cytotoxicity assay against Raji cells. Each panel provides the results comparing the cytotoxicity of the ACE-gdT cells-CD20 (rituximab) cells and that of Control-gdT cells. Cell populations derived from fresh PBMCs of three different donors were tested: FIG. 12A: Donor 1; FIG. 12B: Donor 2; and FIG. 12C: Donor 3.

FIGS. 13A-13C provide results of the cytotoxicity assay against Daudi cells. Each panel provides the results comparing the cytotoxicity of the ACE-gdT cells-CD20 (rituximab) cells and that of Control-gdT cells. Cell populations derived from fresh PBMCs of three different donors were tested: FIG. 13A: Donor 1; FIG. 13B: Donor 2; and FIG. 13C: Donor 3.

FIGS. 14A-14C provide results of the cytotoxicity assay against Raji cells. Each panel provides the results comparing the cytotoxicity of the ACE-gdT cells-CD20 (rituximab) cells and that of Control-gdT cells. Cell populations derived from cryopreserved PBMCs of three different donors were tested: FIG. 14A: Donor 1; FIG. 14B: Donor 2; and FIG. 14C: Donor 3.

FIGS. 15A-15C provide results of the cytotoxicity assay against Daudi cells. Each panel provides the results comparing the cytotoxicity of the ACE-gdT cells-CD20 (rituximab) cells and that of Control-gdT cells. Cell populations derived from cryopreserved PBMCs of three different donors were tested: FIG. 15A: Donor 1; FIG. 15B: Donor 2; and FIG. 15C: Donor 3.

FIGS. 16A-16B present the total cell numbers of the cell populations on different days of the culture. FIG. 16A: Batch 1; FIG. 16B: Batch 2.

FIGS. 17A-17B provide results of the cytotoxicity assay against Raji cells. Each panel provides the results comparing the cytotoxicity of the cell populations cultured in either 5 vol % HPL or 20 vol % HPL. FIG. 17A: Control-gdT cells; FIG. 17B: ACE-gdT cells-CD20 (rituximab).

FIGS. 18A-18B are line graphs showing the total cell numbers (FIG. 18A) and cell viability (FIG. 18B) of the cell populations cultured in either G-Rex (air-permeable) or T-flask (air-impermeable).

FIGS. 19A-19C provide results from mouse model studies demonstrating the anti-tumor activities of both Control-gdT cells and ACE-gdT cells-CD20s. FIG. 19A provides the fluorescent images of tumor cells in mice. FIG. 19B provides the statistical analysis. FIG. 19C provides the survival curves.

Note: “ET,” “ET ratio,” “E:T,” and “E:T ratio” are used equivalently to mean the ratio of effector cells (“E”) to target cells (“T”).

### 5. DETAILED DESCRIPTION

As understood in the art, T lymphocytes, or T cells, are immune cells that play a central role in cell-mediated immunity. T cells express CD3 and T Cell Receptors (TCR) on the cell surface and can be divided into different subtypes by their distinct surface expression of TCRs. “Alpha beta T cells,” “abT cells,” or “αβ T cells,” are equivalent terms which refer to the T cell subset that express both TCR-α chain and a TCR-β chain. “Gamma delta T cells,” “gdT cells,” or “γδ T cells” are equivalent terms which refer to the T cell subset expressing both TCR-γ chain (e.g., Vγ2, Vγ3, Vγ4, Vγ5, Vγ8, Vγ9, or Vγ11) and TCR-δ chain (e.g., Vδ1, Vδ2, Vδ3, or Vδ5) on cell surface (see Pistoia et al., 2018, Front Immunol. 9: 984.; WO2020117862A1). The activation of abT cells is MHC/HLA dependent; wherein gdT cells are similar to innate immune cells and can be activated in an MHC independent manner without the need for antigen processing.

Each TCR chain contains a variable (V) region, a constant (C) region, a transmembrane region and a cytoplasmic tail. The V region contains an antigen binding site. There are two major subtypes of human gdT cells: one that is dominant in the peripheral blood which primarily expresses the delta variable 2 chain (Vδ2), and one dominant in non-hematopoietic tissues which primarily expresses the delta variable 1 (Vδ1) chain. Vδ2 gdT cells generally coexpress Vγ9 and account for 50-95% of the peripheral gdT cell.

GdT cells can infiltrate into the tumors and kill a wide range of tumor cells including both solid and hematopoietic tumors. The antitumor function of gdT cells has been observed in different tumors, such as skin cancer, B-cell lymphoma, prostate cancer, melanoma, and mesenchymal glioblastoma. Various aspects of the anti-tumor activities of gdT cells have been observed. In one aspect, gdT cells are known as stress sensors that recognize unconventional antigens including stress molecules expressed by malignant cells and non-peptidic metabolites. For example, gdT cells can express natural killer group 2 member D (NKG2D), and because transformation is one cellular stress that induces the expression of ligands of NKG2D, the binding between NKG2D on gdT cells and NKG2D ligand, for example, MHC class I polypeptide-related sequence A (MICA), provokes target-specific killing of the transformed cells. In addition to NKG2D, the expression of some other NK receptors has also been shown to participate in tumor recognition and activate the anti-cancer function of gdT cells, including CD226 (DNAM-1), natural cytotoxicity-triggering receptor 3 (NCR3; NKp30), and NCR2 (NKp44).

Additionally, human gdT cells express CD16 and participate in inducing antibody-dependent cellular cytotoxicity (ADCC). Expression of TNF receptors on gdT cells, such as TNF-related apoptosis-inducing ligand (TRAIL) and Fas ligand (FASL), can also kill tumor cells. The anti-tumor activities of gdT cells are also reflected in cytokine production. Proinflammatory cytokines produced by gdT cells, such as IFNγ and TNFα, can further activate antitumor immunity by inducing MHC molecules on the tumor cell surface or by affecting other immune cells. The upregulation of cytotoxic molecules such as granzymes (e.g., granzyme B) and perform can directly kill tumor cells. gdT cells can promote B cells to produce IgE, which has an antitumor effect. As such, gdT cells, especially gdT cells with NK-like properties, hold great promise in cancer immunotherapies.

Human gdT cells normally comprise only 1-5% of circulating T lymphocytes. Despite the increasing interest, gdT cells based-cancer immunotherapies have met with limited clinical success (Yazdanifar et al., 2020, Cells. 9(5):1305; Kabelitz et al., 2020. Cell Mol Immunol. 17(9):925-939; Wu et al., Int J Biol Sci. 10(2):119-35). One limiting factor is that the methods that are currently available to expand the gdT cells are either too time-consuming or ineffective of obtaining gdT cells with sufficient number, purity, and/or potency. Thus, methods to selectively expand a specific subset gdT cell with potent anti-tumor activity are in need.

Provided herein are methods that allow efficient production of cell populations or pharmaceutical compositions enriched in gdT cells with NK-like properties. The gdT cells of the cell populations provided herein have high cytotoxic activities and great therapeutic potential in the treatment of certain diseases and disorders, such as cancers, infectious diseases, and autoimmune diseases.

Before the present disclosure is further described, it is to be understood that the disclosure is not limited to the particular embodiments set forth herein, and it is also to be understood that the terminology used herein is for the purpose of describing particular embodiments, and is not intended to be limiting.

Unless otherwise defined herein, scientific and technical terms used in the present disclosures shall have the meanings that are commonly understood by those of ordinary skill in the art. Further, unless otherwise required by context, singular terms shall include pluralities and plural terms shall include the singular. Generally, nomenclatures used in connection with, and techniques of, cell and tissue culture, molecular biology, immunology, microbiology, genetics and protein and nucleic acid chemistry and hybridization described herein are those well-known and commonly used in the art.

### 5.1 Methods of Production

Provided herein are methods of manufacturing a cell population enriched in gdT cells, comprising culturing a source cell population in a medium supplemented with (i) a phosphoantigen, (ii) a cytokine, and (iii) human platelet lysate (“HPL”). In some embodiments, the culturing is performed under conditions sufficient to activate and expand gdT cells. In some embodiments, the culturing is performed ex vivo. In some embodiments, the culturing is performed in vitro.

As used herein, the term “source cell population” refers to a plurality of cells obtained by isolation directly from a suitable source. The source can be a natural source. For example, the source cell population can be human peripheral blood, or a non-hematopoietic issue. The source cell population can be subsequently cultured ex vivo to prepare a desired cell population. For example, a source cell population can be purified to homogeneity, substantial homogeneity, or to deplete one or more cell types (e.g., ab T cells) by various culture techniques and/or negative or positive selection for a specified cell type. A source cell population can also be cultured to enrich a specific subpopulation. As used herein, a cell population that is “enriched in gdT cells” has a greater percentage of gdT cells than the source cell population from which the cell population is derived. In some embodiments, the cell population enriched in gdT cells can have at least 50%, at least 60%, at least 70%, at least 80%, or at least 90% gdT cells. A cell population enriched in gdT cells can also have less than 50% gdT cells, if the percentage of gdT cells is increased compared to that of the source cell population from which the cell population is derived.

The methods provided herein comprise culturing a source cell population under conditions and for sufficient time to produce a cell population enriched in gdT cells with NK-like properties. In some embodiments of the methods provided herein, the cell population is cultured for at least 4 days, e.g., at least 6 days, at least 7 days, at least 8 days, at least 9 days, at least 10 days, at least 11 days, at least 12 days, at least 13 days, at least 14 days, at least 18 days, at least 21 days, at least 28 days, or longer e.g., about 30 days, about 35 days, about 40 days, about 45 days, or about 50 days. In some embodiments, the methods comprise culturing the cell population for at least 7 days, such as at least 10 days, at least 11 days, at least 14 days, or at least 16 days. In some embodiments of the methods provided herein, the cell population is cultured for 4 to 40 days, 7 to 35 days, 7 to 28 days, or 7 to 21 days, 7 to 18 days, 10 to 30 days, 12 to 20 days, or 14 to 18 days. In some embodiments, the cell population is cultured for 4 to 25 days. In some embodiments, the cell population is cultured for 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 days. In some embodiments, the cell population is cultured for cultured for 4, 7, 10, 12, 14, 17, 22, or 25 days. In some embodiments, the cell population can be cultured for 12 days. The cell population can be cultured for 13 days. The cell population can be cultured for 14 days. The cell population can be cultured for 15 days. The cell population can be cultured for 16 days. The cell population can be cultured for 17 days. The cell population can be cultured for 18 days. The cell population can be cultured for 19 days. The cell population can be cultured for 20 days. The cell population can be cultured for about 25 days. The cell population can be cultured for about 30 days. The cell population can be cultured for about 35 days. The cell population can be cultured for about 40 days. The cell population can be cultured for about 45 days. The cell population can be cultured for about 50 days.

TCRα/β T cells, or abT cells are known to induce graft versus host response in adoptive cell therapies. Excluding abT cells from the engrafted cell population reduces or prevents the development of GvHD in adoptive cell therapy. In some embodiments, methods provided herein further comprise depleting abT cells. The abT cells can be depleted at different time during the culture. In some embodiments, the abT cells are depleted at the beginning of the culture. In some embodiments, the abT cells are depleted at the end of the culture. In some embodiments, the abT cells are depleted in the first half of the culture. In some embodiments, the abT cells are depleted in the second half of the culture. In situations where the source cell population has relatively few T cells. It can be beneficial to allow all cells to expand for a few days before depleting the abT cells. In some embodiments, the abT cells are depleted on Day 2 or later, Day 3 or later, Day 4 or later, Day 5 or later, or Day 6 or later. Additionally, depleting abT cells before their percentages get to certain threshold can help achieve the most efficient expansion of the gdT cells. Accordingly, in some embodiments, the abT cells are depleted before they reach 30%, 25%, 20%, 15%, 12%, 10%, 9%, or 8% of the cell population. It is generally observed that, if not depleted, the abT cell percentage would increase in the first 20 days of culture. Thus, in some embodiments, the abT cells are depleted before Day 14, before Day 12, before Day 10, before Day 9, before Day 8, or before Day 4 of the culture. In some embodiments, the abT cells are depleted around the half-time of the culture. For example, in some embodiments, the cells are cultured for 30 to 40 days and the abT cells are depleted between Day 18 and Day 25. In some embodiments, the cells are cultured for 14 to 18 days and the abT cells are depleted between Day 4 and Day 10. In some embodiments, the cells are cultured for about 14 to 18 days, and the abT cells are depleted on Day 6 or Day 7. In some embodiments, the abT cells are depleted on Day 4, 5, 6, 7, 8, 9, 10, 11, 12, 13, 14, 15, 16, 17, or 18. In some embodiments, the abT cells are depleted on Day 7, 8, 9, 10, 12, 14 or 16. In some embodiments, the abT cells are depleted on Day 4, 5, 6, 7, or 8. In some embodiments, the abT cells are depleted on Day 6. In some embodiments, the abT cells are depleted on Day 7. In some embodiments, the abT cells are depleted on Day 8. In some embodiments, the cell populations are further cultured for 3-25 days after the depletion of the abT cells. In some embodiments, the cell populations are further cultured for 3, 5, 7, 9, 11, 13, 15, 17, 19, 21, 23 or 25 days after the depletion of abT cells.

The culture media used in the methods described herein can be supplemented with (i) a phosphoantigen. As understood in the art, a “phosphoantigen” is a T cell agonist, more particularly a gdT cell agonist, whose activity depends on the presence of a phosphate moiety. It is also known in the art that certain phosphoantigen can specifically activate gdT cells. (Espinosa et al., Microbes and Infections 2001; Belmant et al., Drug discovery today 2005; US20100189681A1). In some embodiments, the phosphoantigen is a bisphosphonate. In some embodiments, the bisphosphonate used in methods described herein is selected from the group consisting of clodronate, etidronate, alendronate, pamidronate, zoledronate (zoledronic acid), neridronate, ibandronate, and pamidronate. In some embodiments, the bisphosphonate used in methods described herein is zoledronate.

In some embodiments, the phosphoantigen used in methods described herein is selected from the group consisting of bromohydrin pyrophosphate (BrHPP), 4-hydroxy-but-2-enyl pyrophosphate (HMBPP), isopentenyl pyrophosphate (IPP), and dimethylallyl pyrophosphate (DMAPP).

The phosphoantigen is supplemented at a concentration of 0.1-20 μM in the medium. In some embodiments, the phosphoantigen is supplemented at a concentration of about 0.1, 0.5, 1, 1.5, 2, 3, 3.5, 4, 5, 5.5, 6, 6.5, 7, 7.5, 8, 8.5, 9, 10, 11, 11.5, 12, 13, 13.5, 14, 15, 16, 17, 18, or 19 μM. In some embodiments, the phosphoantigen is supplemented at about 0.1 μM. The phosphoantigen can be supplemented at about 0.5 μM. The phosphoantigen can be supplemented at about 1 μM. The phosphoantigen can be supplemented at about 1.5 μM. The phosphoantigen can be supplemented at about 2 μM. The phosphoantigen can be supplemented at about 3 μM. The phosphoantigen can be supplemented at about 4 μM. The phosphoantigen can be supplemented at about 5 μM. The phosphoantigen can be supplemented at about 6 μM. The phosphoantigen can be supplemented at about 7 μM. The phosphoantigen can be supplemented at about 8 μM. The phosphoantigen can be supplemented at about 9 μM. The phosphoantigen can be supplemented at about 10 μM. The phosphoantigen can be supplemented at about 12 μM. The phosphoantigen can be supplemented at about 15 μM. The phosphoantigen can be supplemented at about 18 μM. The phosphoantigen can be supplemented at about 20 μM. The phosphoantigen can be any phosphoantigen disclosed herein or otherwise known in the art.

In some embodiments, the phosphoantigen used in methods described herein is zoledronate, which is supplemented at a concentration of 0.1-20 μM in the medium. In some embodiments, the zoledronate is supplemented at a concentration of about 0.1, 0.5, 1, 1.5, 2, 3, 3.5, 4, 5, 5.5, 6, 6.5, 7, 7.5, 8, 8.5, 9, 10, 11, 11.5, 12, 13, 13.5, 14, 15, 16, 17, 18, or 19 μM. In some embodiments, the zoledronate is supplemented at about 0.1 μM. The zoledronate can be supplemented at about 0.5 μM. The zoledronate can be supplemented at about 1 μM. The zoledronate can be supplemented at about 1.5 μM. The zoledronate can be supplemented at about 2 μM. The zoledronate can be supplemented at about 3 μM. The zoledronate can be supplemented at about 4 μM. The zoledronate can be supplemented at about 5 μM. The zoledronate can be supplemented at about 6 μM. The zoledronate can be supplemented at about 7 μM. The zoledronate can be supplemented at about 8 μM. The zoledronate can be supplemented at about 9 μM. The zoledronate can be supplemented at about 10 μM. The zoledronate can be supplemented at about 12 μM. The zoledronate can be supplemented at about 15 μM. The zoledronate can be supplemented at about 18 μM. The zoledronate can be supplemented at about 20 μM.

The culture media used in the methods described herein can be supplemented with (ii) a cytokine. Cytokines include interleukins, lymphokines, interferons, colony stimulating factors and chemokines. In one embodiment, the cytokine is selected from the group consisting of interleukin-2 (IL-2), interleukin-4 (IL-4), interleukin-6 (IL-6), interleukin-7 (IL-7), interleukin-8 (IL-8), interleukin-9 (IL-9), interleukin-12 (IL-12), interleukin-15 (IL-15), interleukin-18 (IL-18), interleukin-21 (IL-21), interleukin-33 (IL-33), insulin-like growth factor 1 (IGF-1), interleukin-1 b (IL-1b), interferon-gamma (IFN-g) and stromal cell-derived factor-1 (SDF-1). Compounds that have the same activity as the cytokines with respect to its ability to promote similar physiological effects on gdT cells in culture can also be used in methods disclosed herein, including, for example, cytokine mimetics.

In some embodiments, the cytokine can be IL-2, IL-4, IL-6, IL-7, IL-8, IL-9, IL-12, IL-15, IL-18, IL-21, IL-33, or any combination thereof. In some embodiments, the cytokine is IL-2.

In some embodiments, more than one cytokine can be used. The cytokines can be simultaneously supplemented to the culture media or added at different times. In some embodiments of the methods disclosed herein, the culture media can be supplemented with a combination of at least two different cytokines during the culture. In some embodiments of the methods disclosed herein, the culture media can be supplemented with a first cytokine in the beginning at the culture and with a second cytokine at a later time during the culture. The first and second cytokines can be independently selected from the group consisting of interleukins, lymphokines, interferons, colony stimulating factors and chemokines. In some embodiments, the first and second cytokines can be independently selected from the group consisting of IL-2, IL-4, IL-6, IL-7, IL-8, IL-9, IL-12, IL-15, IL-18, IL-21, and IL-33.

The cytokine used in methods described herein can be of human or animal origin. In some embodiments, the cytokine is of human origin. It can be a wildtype protein or any biologically active fragment or variant that maintains the activity of the wildtype protein to promote similar physiological effects on gdT cells in culture. The cytokines can be in soluble form, fused or complexed with another molecule, such as for example a peptide, polypeptide or biologically active protein. In some embodiments, a human recombinant cytokine is used.

In some embodiments, the methods disclosed herein comprise using a culture medium supplemented with a cytokine at a concentration ranging between 1-10000 U/ml. In some embodiments, cytokine concentration can range between 100-1000 U/ml. In some embodiments, the cytokine is supplemented at a concentration of 100-2500 IU/mL in the media. In some embodiments, the cytokine is supplemented at a concentration of 200-3000 IU/mL in the media. In some embodiments, the cytokine is supplemented at a concentration of about 100, about 150, about 200, about 250, about 300, about 350, about 400, about 450, about 500, about 550, about 600, about 650, about 700, about 750, about 800, about 850, about 900, about 950, about 1000, about 1100, about 1200, about 1300, about 1400, about 1500, about 1600, about 1700, about 1800, about 1900, about 2000, about 2100, about 2200, about 2300, about 2400, about 2500, about 2600, about 2700, about 2800, about 2900, or about 3000 IU/mL. In some embodiments, the cytokine is supplemented at a concentration of about 100 IU/mL. The cytokine can be supplemented at a concentration of about 200 IU/mL. The cytokine can be supplemented at a concentration of about 350 IU/mL. The cytokine can be supplemented at a concentration of about 500 IU/mL. The cytokine can be supplemented at a concentration of about 700 IU/mL. The cytokine can be supplemented at a concentration of about 1000 IU/mL. The cytokine can be supplemented at a concentration of about 1500 IU/mL. The cytokine can be supplemented at a concentration of about 2000 IU/mL.

A person of ordinary skill in the art would understand that a different unit can be used to characterize the cytokine concentration in the culture media. In some embodiments, the cytokine is supplemented at a concentration of 0.0612-1.53 μg/mL in the media. In some embodiments, the cytokine is supplemented at a concentration of 0.05-5 μg/mL in the media. In some embodiments, the cytokine is supplemented at a concentration of about 0.05, about 0.06, about 0.07, about 0.08, about 0.09, about 0.1, about 0.2, about 0.3, about 0.4, about 0.5, about 0.6, about 0.7, about 0.8, about 0.9, about 1.0, about 1.1, about 1.2, about 1.3, about 1.4, about 1.5, about 1.6, about 1.7, about 1.8, about 1.9 μg, about 2.0 μg, about 2.2, about 2.4, about 2.6, about 2.8, about 3.0, about 3.2, about 3.4, about 3.6, about 3.8, about 4.0, about 4.2, about 4.4, about 4.6, about 4.8, or about 5.0 μg/mL. In some embodiments, the cytokine is supplemented at about 0.1 μg/mL. The cytokine can be supplemented at about 0.2 μg/mL. The cytokine can be supplemented at about 0.3 μg/mL. The cytokine can be supplemented at about 0.4 μg/mL. In some embodiments, the cytokine is supplemented at about 0.5 μg/mL. In some embodiments, the cytokine is supplemented at about 1.0 μg/mL. In some embodiments, the cytokine is supplemented at about 1.5 μg/mL. In some embodiments, the cytokine is supplemented at about 2 μg/mL.

The cytokine can be any cytokine disclosed herein or otherwise known in the art. When at least two cytokines are used, the cytokines are supplemented at a total concentration of about 100, about 150, about 200, about 250, about 300, about 350, about 400, about 450, about 500, about 550, about 600, about 650, about 700, about 750, about 800, about 850, about 900, about 950, about 1000, about 1100, about 1200, about 1300, about 1400, about 1500, about 1600, about 1700, about 1800, about 1900, about 2000, about 2100, about 2200, about 2300, about 2400, about 2500, about 2600, about 2700, about 2800, about 2900, or about 3000 IU/mL in the media. In some embodiments, the cytokines are supplemented at a total concentration of about 0.05, about 0.06, about 0.07, about 0.08, about 0.09, about 0.1, about 0.2, about 0.3, about 0.4, about 0.5, about 0.6, about 0.7, about 0.8, about 0.9, about 1.0, about 1.1, about 1.2, about 1.3, about 1.4, about 1.5, about 1.6, about 1.7, about 1.8, about 1.9 μg, about 2.0 μg, about 2.2, about 2.4, about 2.6, about 2.8, about 3.0, about 3.2, about 3.4, about 3.6, about 3.8, about 4.0, about 4.2, about 4.4, about 4.6, about 4.8, or about 5.0 μg/mL μg/mL in the media.

In some embodiments, IL-2 is used, and the methods disclosed herein comprise using a culture medium supplemented with IL-2 at a concentration ranging between 1-10000 U/ml. In some embodiments, IL-2 concentration can range between 100-1000 U/ml. In some embodiments, IL-2 is supplemented at a concentration of 100-2500 IU/mL in the media. In some embodiments, IL-2 is supplemented at a concentration of 200-3000 IU/mL in the media. In some embodiments, IL-2 is supplemented at a concentration of about 100, about 150, about 200, about 250, about 300, about 350, about 400, about 450, about 500, about 550, about 600, about 650, about 700, about 750, about 800, about 850, about 900, about 950, about 1000, about 1100, about 1200, about 1300, about 1400, about 1500, about 1600, about 1700, about 1800, about 1900, about 2000, about 2100, about 2200, about 2300, about 2400, about 2500, about 2600, about 2700, about 2800, about 2900, or about 3000 IU/mL. In some embodiments, IL-2 is supplemented at a concentration of about 100 IU/mL. IL-2 can be supplemented at a concentration of about 200 IU/mL. IL-2 can be supplemented at a concentration of about 350 IU/mL. IL-2 can be supplemented at a concentration of about 500 IU/mL. IL-2 can be supplemented at a concentration of about 700 IU/mL. IL-2 can be supplemented at a concentration of about 1000 IU/mL. IL-2 can be supplemented at a concentration of about 1500 IU/mL. IL-2 can be supplemented at a concentration of about 2000 IU/mL.

A person of ordinary skill in the art would understand that a different unit can be used to characterize TL-2 concentration in the culture media. In some embodiments, IL-2 is supplemented at a concentration of 0.0612-1.53 μg/mL in the media. In some embodiments, IL-2 is supplemented at a concentration of 0.05-5 μg/mL in the media. In some embodiments, IL-2 is supplemented at a concentration of about 0.05, about 0.06, about 0.07, about 0.08, about 0.09, about 0.1, about 0.2, about 0.3, about 0.4, about 0.5, about 0.6, about 0.7, about 0.8, about 0.9, about 1.0, about 1.1, about 1.2, about 1.3, about 1.4, about 1.5, about 1.6, about 1.7, about 1.8, about 1.9 μg, about 2.0 μg, about 2.2, about 2.4, about 2.6, about 2.8, about 3.0, about 3.2, about 3.4, about 3.6, about 3.8, about 4.0, about 4.2, about 4.4, about 4.6, about 4.8, or about 5.0 μg/mL. In some embodiments, IL-2 is supplemented at about 0.1 μg/mL. IL-2 can be supplemented at about 0.2 μg/mL. IL-2 can be supplemented at about 0.3 μg/mL. IL-2 can be supplemented at about 0.4 μg/mL. In some embodiments, IL-2 is supplemented at about 0.5 μg/mL. In some embodiments, IL-2 is supplemented at about 1.0 μg/mL. In some embodiments, IL-2 is supplemented at about 1.5 μg/mL. In some embodiments, IL-2 is supplemented at about 2 μg/mL.

The culture media used in the methods described herein can be supplemented with (iii) HPL. HPL is commercially available from StemCell Technologies, Sigma Aldrich, Millipore, etc. The HPL can be supplemented in the media at a concentration of 0.5-30 vol %. In some embodiments, the HPL is supplemented at a concentration of 1-20 vol %. In some embodiments, the HPL is supplemented at a concentration of 5-20 vol %. In some embodiments, the HPL is supplemented at a concentration of 5-15 vol %. In some embodiments, the HPL is supplemented in the culture media at a concentration of about 0.5%, about 1%, about 1.5%, about 1.6%, about 2%, about 2.5%, about 2.6%, about 3%, about 3.5%, about 3.6%, about 4%, about 4.5%, about 4.6%, about 5.0%, about 5.1%, about 5.5%, about 5.6%, about 6%, about 6.1%, about 6.5%, about 6.6%, about 7%, about 7.1%, about 7.5%, about 7.6%, about 8%, about 8.1%, about 8.5%, about 8.6%, about 9%, about 9.1%, about 9.5%, about 9.6%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about or 30% (volume percent, vol %, or % (v/v)). In some embodiments, the HPL is supplemented in the culture media at a concentration of about 5%. The HPL concentration can be about 2%. The HPL concentration can be about 3%. The HPL concentration can be about 4%. The HPL concentration can be about 6%. The HPL concentration can be about 7%. The HPL concentration can be about 8%. The HPL concentration can be about 9%. The HPL concentration can be about 10%. The HPL concentration can be about 12%. The HPL concentration can be about 15%. The HPL concentration can be about 18%. The HPL concentration can be about 20%. The HPL concentration can be about 25%. The HPL concentration can be about 30%.

In some embodiments, the culture media used in methods described herein can be serum-free. In some embodiments, the culture media can be a serum replacement medium, such as a chemically defined medium that avoids the use of human or animal derived serum. Samples cultured in serum-free media have the advantage of avoiding issues with filtration, precipitation, contamination and supply of serum.

Numerous basal culture media suitable for use in the proliferation of gdT cells are available, such as Iscoves medium and RPMI-1640 (available form Gibco, Sigma Aldrich, Biological Industries, STEMCELL Technologies, Life Technologies; etc.), AIM-V, X-VIVO 10, X-VIVO 15 or X-VIVO 20 (Lonza). The culture media can be supplemented with other media factors as defined herein. The culture media used in the methods described herein can further comprise other components useful for the expansion and/or active of gdT cells. Examples of other ingredients that can be added, include, but are not limited to, purified proteins such as albumin, a lipid source such as low density lipoprotein (LDL), vitamins, amino acids, steroids and any other supplements supporting or promoting cell growth and/or survival.

In some embodiments, the culture media used in the methods described herein comprise glucose at a concentration of 600-5000 mg/L. The culture media can have a glucose content of from about 500 mg/L to about 1000 mg/L, from about 500 mg/L to about 1500 mg/L, from about 500 mg/L to about 2000 mg/L, from about 750 mg/L to about 1000 mg/L, from about 750 mg/L to about 1500 mg/L, from about 750 mg/L to about 2000 mg/L, from about 1000 mg/L to about 1500 mg/L, from about 1000 mg/L to about 2000 mg/L, from 1000 mg/L to 3000 mg/L, or from 1000 mg/L to 4000 mg/L. In some embodiments, the cells can be maintained in culture medium having a glucose content of about 1250 mg/L. In some cases, such as where a high cell density culture is maintained, cells can be maintained in culture medium having a glucose content of about 1000 mg/L to about 5000 mg/L, from about 1000 mg/L to about 4000 mg/L, from about 2000 mg/L to about 5000 mg/L, or from about 2000 mg/L to about 4000 mg/L. In some embodiments, the medium comprises glucose at a concentration of 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, or 4900 mg/L.

In some embodiments of the methods described herein, the medium can be changed during the culture. As known in the art, medium change refers to the procedure wherein the old culture medium in the culturing device is removed and fresh medium added. The culture medium can be changed in half. The culture medium can also be changed in its entirety. The culture medium can be changed once per week, twice per week, three times per week, every other day, or daily. In some embodiments, the culture medium can be changed every two days or every three days.

In some embodiments, the cells are reseeded with fresh culture medium during the culture. Generally, the cells are reseeded to be diluted or adjusted to a density that supports further expansion. The cells can be reseeded once or multiple times during the culture. In some embodiments, cells can be reseeded once per week, twice per week, three times per week, every other day, or daily. In some embodiments, cells can be reseeded at least once, at least twice, at least three times, at least four times, or at least five times during the culture. In some embodiments, the cells are reseeded every two days or every three days. The entire culture period can include medium change on certain days and reseeding on different days. In some embodiments, cell density is adjusted to a range of from about 0.5×10⁶ to about 1×10⁶ cells/mL, from about 0.5×10⁶ to about 1.5×10⁶ cells/mL, from about 0.5×10⁶ to about 2×10⁶ cells/mL, from about 0.75×10⁶ to about 1×10⁶ cells/mL, from about 0.75×10⁶ to about 1.5×10⁶ cells/mL, from about 0.75×10⁶ to about 2×10⁶ cells/mL, from about 1×10⁶ to about 2×10⁶ cells/mL, or from about 1×10⁶ to about 1.5×10⁶ cells/mL, from about 1×10⁶ to about 2×10⁶ cells/mL, from about 1×10⁶ to about 3×10⁶ cells/mL, from about 1×10⁶ to about 4×10⁶ cells/mL, from about 1×10⁶ to about 5×10⁶ cells/mL, from about 1×10⁶ to about 10×10⁶ cells/mL, from about 1×10⁶ to about 15×10⁶ cells/mL, from about 1×10⁶ to about 20×10⁶ cells/mL, or from about 1×10⁶ to about 30×10⁶ cells/mL. A person of ordinary skill in the art would be able to optimize the medium change procedure (frequency, timing, amount being changed, etc.) as a routine practice.

Generally, during medium change or reseeding, the fresh medium is supplemented with the same constituents as the medium used in the beginning of the culture, including the phosphoantigen (e.g., zoledronate), the cytokine (e.g., IL-2) and the HPL. In some embodiments of the methods described herein, the fresh culture medium used for medium change or reseeding is not supplemented with zoledronate. In some embodiments of the methods described herein, phosphoantigen (e.g., zoledronate) is only supplemented in the culture medium used in the beginning of the culture. In some embodiments of the methods described herein, phosphoantigen (e.g., zoledronate) is not supplemented in the culture medium used toward the end of the culture. For example, in some embodiments, phosphoantigen (e.g., zoledronate) is not supplemented in the culture medium used on the last day, the last two days, the last three days, the last quarter, the last third, or the second half of the culture period. In some embodiments of the methods described herein that comprise depleting the abT cells, phosphoantigen (e.g., zoledronate) is supplemented in culture media used before abT depletion, but not supplemented after the abT depletion.

In some embodiments of the methods described herein, the cytokine (e.g., IL-2) is replenished during the culture. The cytokine (e.g., IL-2) can be replenished on days with no medium change or reseeding. In some embodiments, the cytokine (e.g., IL-2) can be replenished once per week, twice per week, three times per week, every other day, or daily.

In some embodiments, cells are cultured at 37° C. in a humidified atmosphere containing 5% CO₂ in a suitable medium during the culture.

For illustrative purposes, the methods described herein comprise culturing the cells for 16 days and include the following procedures:

| Day | Procedure |
|---|---|
| 1 | beginning culture in complete medium |
| 2 | cytokine replenishment |
| 4 | medium change with complete medium |
| 6 | depleting abT cells and reseeding in complete |
| | medium (no zoledronate) |
| 8 | reseeding in complete medium (no zoledronate) |
| 9 | cytokine replenishment |
| 10 | reseeding in complete medium (no zoledronate) |
| 11 | cytokine replenishment |
| 13 | reseeding in complete medium (no zoledronate) |
| 14 | medium change with complete medium (no zoledronate) |
| 16 | harvesting resulting cell population |

As illustrated, in the exemplary 16-day culture procedure, abT cells are depleted on Day 6. The complete culture medium is supplemented with cytokine (e.g., 350 or 700 IU/mL IL-2) phosphoantigen (e.g., 1 μM zoledronate), and HPL (e.g., 5 vol %). The cytokine is supplemented about every day or every other day by either direct replenishment, medium change, or reseeding, whereas the phosphoantigen (e.g., 1 μM zoledronate) is only supplemented in the culture media before the abT depletion. A person of ordinary skill in the art would understand that the illustrated procedure can be modified and further optimized as routine practice.

The source cell populations comprising gdT cells can be obtained from a variety of samples. In some embodiments, the sample is a hematopoietic sample or fraction thereof (i.e., the source cell population is obtained from a hematopoietic sample or a fraction thereof). Hematopoietic samples include blood (such as peripheral blood or umbilical cord blood), bone marrow, lymphoid tissue, lymph node tissue, thymus tissue, and fractions or enriched portions thereof. In some embodiments, the sample is blood sample. In some embodiments, the source cell population can be obtained from umbilical cord blood or fractions thereof. In some embodiments, the source cell population can be obtained from peripheral blood or fractions thereof. In some embodiments, the source cell population can be obtained from fractions of peripheral blood, such as buffy coat cells, leukapheresis products, peripheral blood mononuclear cells (PBMCs) and low density mononuclear cells (LDMCs). In some embodiments, the source cell population comprise PBMCs. In some embodiments, the sample is human blood or a fraction thereof. The cells can be obtained from a sample of blood using techniques known in the art such as density gradient centrifugation. PBMCs can be collected from a subject, for example, with an apheresis machine, such as the Ficoll-Paque™ PLUS (GE Healthcare) system.

In some embodiments, the source cell populations can be obtained from a non-hematopoietic tissue sample. Non-hematopoietic tissue is a tissue other than blood, bone marrow, lymphoid tissue, lymph node tissue, or thymus tissue. In some embodiments, the source cell population is not obtained from particular types of samples of biological fluids, such as blood or synovial fluid. Non-hematopoietic tissues include, but are not limited to, those from the gastrointestinal tract (e.g., colon or gut), mammary gland, lung, prostate, liver, spleen, pancreas, uterus, vagina and other cutaneous, mucosal or serous membranes. Methods to obtain source cell population from non-hematopoietic tissue samples are known in the art. For example, the source cell populations can be obtained from the non-hematopoietic tissue sample by culturing the non-hematopoietic tissue sample on a synthetic scaffold configured to facilitate cell egress from the non-hematopoietic tissue sample.

GdT cells can also be resident in cancer tissue samples. In some embodiments, the source cell population can be obtained from human cancer tissue samples (e.g., hematological cancer tissues or solid tumor tissues). The cancer tissue sample can be, e.g., tumors of the breast or prostate. In other embodiments, the source cell population can be from a sample other than human cancer tissue (e.g., a tissue without a substantial number of tumor cells). For example, the source cell population can be from a region of healthy tissue separate from a nearby or adjacent cancer tissue.

The source cell population can be obtained from human or non-human animal tissue. In some embodiments, methods described herein further comprise obtaining the source cell population from human or non-human animal tissue. In some embodiments, the sample is obtained from a human. In some embodiments, the sample is obtained from a non-human animal subject.

In some embodiments, the cell population prepared according to the methods disclosed herein are used in a transplant. Accordingly, in some embodiments, methods described herein further comprise obtaining the source cell population from a donor. In some embodiments, the donor is a human. The donor can be a healthy human. The donor can be a diseased human. In some embodiments, the recipient of the transplant is a human. The transplant can be an autologous transplant. The transplant can be an allogeneic transplant. As understood in the art, the term “autologous” when used in reference to a material means that the material is derived from the same individual to which it is later to be re-introduced; and the term “allogeneic” when used in reference to a material means that the material is a graft derived from a different individual of the same species. In some embodiments, the source cell populations are obtained from an autologous donor. In some embodiments, the source cell populations are obtained from an allogeneic donor. In some embodiments, the source cell populations are obtained from a healthy allogeneic donor, and the cell populations prepared using the methods described herein are used in a transplant for a cancer patient.

In some embodiments, the source cell population can be obtained from a freshly prepared sample. The source cell population can also be obtained from a cryopreserved sample which is thawed immediately before being cultured in the methods disclosed herein. 37° C. water baths can be used to thaw cryopreserved PBMCs.

In some embodiments, the source cell population comprises PBMCs, and methods described herein comprise obtaining the PBMCs from peripheral blood of a donor. The donor can be an autologous donor. The donor can be an allogeneic donor. The PBMC can be freshly prepared. The PBMC can also be cryopreserved and thawed immediately before being used for the source cell population in the methods disclosed herein.

In some embodiments, methods provided herein can expand the gdT cells in the source cell population for at least 1,000-fold during the culture. In some embodiments, the gdT cells are expanded for at least 500-fold, at least 1,000-fold, at least 2,000 fold, at least 5,000 fold, at least 10,000 fold, at least 15,000 fold, at least 20,000 fold, at least 30,000 fold, at least 40,000 fold, at least 50,000-fold, at least 60,000 fold, at least 70,000-fold, at least 80,000 fold, or at least 100,000-fold during the culture. In some embodiments, the source cell population is derived from a single donor. In some embodiments, the source cell population is derived from more than one donor or multiple donors (e.g., 2, 3, 4, 5, or from 2-5, 2-10, or 5-10 donors, or more). In some embodiments, cell populations produced by the methods provided herein comprise a clinically relevant number (at least 10⁷, at least 10⁸, at least 10⁹, at least 10¹⁰, at least 10¹¹, or at least 10¹², or from about 10⁷ to about 10¹²) of gdT cells from as few as one donor. In some embodiments, the methods described herein can provide a clinically relevant number (e.g., at least 10⁷, at least 10⁸, at least 10⁹, at least 10¹⁰, at least 10¹¹, or at least 10¹², or from about 10⁷ to about 10¹²) of gdT cells within less than 40 days (e.g., about 30 days, about 20 days, about two weeks or about one week) from the time of obtaining the source cell population from a single donor. In some embodiments, the methods described herein can provide a clinically relevant number of gdT cells within less than 30 days from the time of obtaining the source cell population from a single donor. In some embodiments, the methods described herein can provide a clinically relevant number of gdT cells within less than 20 days from the time of obtaining the source cell population from a single donor. In some embodiments, the methods described herein can provide a clinically relevant number of gdT cells within about 2 weeks (e.g., 14-18 days) from the time of obtaining the source cell population from a single donor. In some embodiments, the methods described herein can provide a clinically relevant number of gdT cells within 16 days from the time of obtaining the source cell population from a single donor. In some embodiments, the methods described herein can provide a population of at least 10⁸, at least 10⁹, at least 10¹⁰, or at least 10¹¹ gdT cells within 16 days from the time of obtaining the source cell population from a single donor. In some embodiments, the methods described herein can provide at least 10¹⁰ gdT cells within 16 days from the time of obtaining the source cell population from a single donor.

In some embodiments, methods provided herein of expanding gdT cells can comprise a population doubling time of less than 5 days. In some embodiments, the doubling time for gdT cells during the culture can be less than 4.5 days, less than 4.0 days, less than 3.9 days, less than 3.8 days, less than 3.7 days, less than 3.6 days, less than 3.5 days, less than 3.4 days, less than 3.3 days, less than 3.2 days, less than 3.1 days, less than 3.0 days, less than 2.9 days, less than 2.8 days, less than 2.7 days, less than 2.6 days, less than 2.5 days, less than 2.4 days, less than 2.3 days, less than 2.2 days, less than 2.1 days, less than 2.0 days, less than 46 hours, less than 42 hours, less than 38 hours, less than 35 hours, less than 32 hours, less than 30 hours, less than 29 hours, less than 28 hours, less than 27 hours, less than 26 hours, less than 25 hours, less than 24 hours, less than 23 hours, less than 22 hours, less than 21 hours, less than 20 hours, less than 19 hours, less than 18 hours, less than 17 hours, less than 16 hours, less than 15 hours, less than 14 hours, less than 13 hours, or less than 12 hours.

Methods provided herein result in the enrichment of gdT cells in the cell population. In some embodiments, at least 50% of the resulting cell population are gdT cells. In some embodiments, at least 55%, at least 60%, at least 65%, at least 70%, at least 75%, at least 80%, at least 85%, at least 87%, or at least 90% of the resulting cell population are gdT cells. In some embodiments, at least 90%, at least 91%, at least 92%, at least 93%, at least 94%, at least 95%, at least 96%, at least 97%, at least 98%, or at least 99% of the resulting cell population are gdT cells. In some embodiments, at least 75% of the resulting cell population are gdT cells. In some embodiments, at least 80% of the resulting cell population are gdT cells. In some embodiments, at least 85% of the resulting cell population are gdT cells. In some embodiments, at least 90% of the resulting cell population are gdT cells. In some embodiments, at least 95% of the resulting cell population are gdT cells.

Tab cells are highly reactive and can cause graft v. host diseases, therefore suitable cell populations for administration to patients provided herein only contain low levels of abT cells. In some embodiments, methods provided herein produce cell populations having less than about 10% abT cells, such as less than about 5%, 4%, 3%, 2%, 1.5%, 1%, 0.5%, 0.2%, 0.1% or 0.05% abT cells. In some embodiments, cell populations prepared by methods described herein contain less than about 1% abT cells.

An increase or decrease in expression of cell surface markers can be additionally or alternatively used to characterize the cell populations prepared by methods described herein, including, for example, CD69. In some embodiments, a larger percentage of gdT cells of the cell populations prepared by methods described herein expresses of CD69, relative to the source population prior to expansion. For example, in some embodiments, more than about 30%, such as more than about 35%, 40%, 45%, 50%, 55%, 60%, 65%, 70%, 75%, 80%, 85%, 90% of gdT cells of the cell populations prepared by methods described herein expresses of CD69. In some embodiments, the cell populations prepared by methods described herein have a greater mean expression of CD69, relative to the source cell population. In some embodiments, the cell populations prepared by methods described herein express a low level of PD-1 and/or TIM-3. More details regarding the surface markers are described in Section 5.2 below.

In some embodiments, methods provided herein further comprise adding a targeting moiety to the surface of the cells in the resulting cell population. The targeting moiety as used herein exhibit specific binding to a biological marker on a target cell. In some embodiments, the targeting moiety is complexed to the cell surface via the interaction between a first linker conjugated to the targeting moiety and a second linker conjugated to the cell. In some embodiments, the targeting moiety is exogenously expressed on the surface of gdT cells provided herein as the extracellular domain of a receptor protein, such as a chimeric antigen receptor (“CAR”) or a T cell receptor (“TCR”). Accordingly, in some embodiments, methods provided herein further comprise introducing a nucleic acid encoding a CAR or TCR to the gdT cells. See sections 5.2.1 to 5.2.3 below for further details.

In some embodiments of the methods described herein, the cells are cultured in an air-permeable device. The air-permeable devices, or air-permeable cell culture device, are containers for tissue culture equipped an air-permeable surface. In some embodiments, the cells can be seeded on such air-permeable surface. In some embodiments, the air-permeable device is a G-Rex device. As known in the art, a G-Rex device is a cell culture flask with an air-permeable membrane at the base that supports large media volumes without compromising gas exchange (Bajgain et al., 2014, Molecular Therapy-Methods & Clinical Development, 14015). In some embodiments, the air-permeable device can be a bioreactor. In some embodiments, the bioreactor can be a WAVE bioreactor. In some embodiments, the bioreactor can be a stirred tank bioreactor.

Some methods currently used in the art to expand gdT cells include the step of culturing the gdT cells with a feeder cell, or an antigen from a microbial pathogen, such as certain bacterial components. Feeder cells can be allogeneic PBMCs, or transformed cells (e.g., EBV-transformed lymphoblastic cell lines), or both. The bacterial component can be, for example, Mycobacterium tuberculosis low molecular peptide antigen (Mtb-Ag), Staphylococcal enterotoxin A (SEA) and Streptococcal protein A. The use of either feeder cells or pathogenic component poses a potential risk to recipients of the cells. Thus, it is critical to ensure that feeder cells, bacterial component, or any foreign substance that might be harmful for a potential transplant recipient are removed prior to clinical administration. Feeder cells must be cultivated in parallel and irradiated before use; if irradiation is insufficient, feeder cells might overgrow gdT cells, contaminating the cell preparation. Ex vivo culture free of any feeder cell and microbial pathogen is advantageous, as it simplifies the culturing procedure. Also, less handling lowers the risk of contamination introduced during cultivation. Thus, the generation of clinically relevant numbers of gdT cells without the use of feeders or microbial pathogens is more cost-effective as well as safer. Methods provided herein are capable of producing a clinically relevant number of gdT cells with sufficient activity without the need to use feeder cells or microbial pathogens. Accordingly, in some embodiments, methods provided herein do not use feeder cells or microbial pathogen such as bacterial components to stimulate the proliferation and/or activity of the gdT cells.

Some methods of enriching gdT cells ex vivo include positively selecting the gdT cells. As understood in the art, positive selection refers to the procedure that involves using a positive feature of the desired cell population (such as the expression of a surface marker) to select targeted cells. Cells without such positive feature are discarded. For example, positive selection for gdT cells in a cell population can use, e.g., beads conjugated with antibodies against TCRVδ2+ to capture the gdT cells. Unbound cells are discarded. Positive selection can be used to prepare cell populations with high purity of the desired cell type. However, the extra step of positive selection and collateral loss of desired cell type (e.g., gdT) could also comprise the quality of the resulting cell population. Methods provided herein allows preparation of cell populations with gdT cells of high purity without using positive selection. Accordingly, in some embodiments, methods provided herein do not include positive selection for gdT cells. In some embodiments, methods provided herein do not include any positive selection.

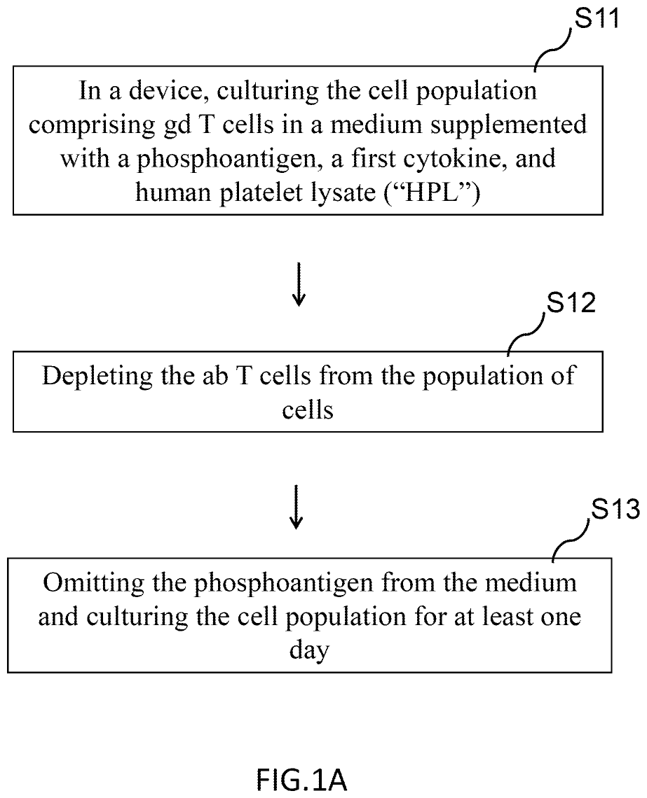

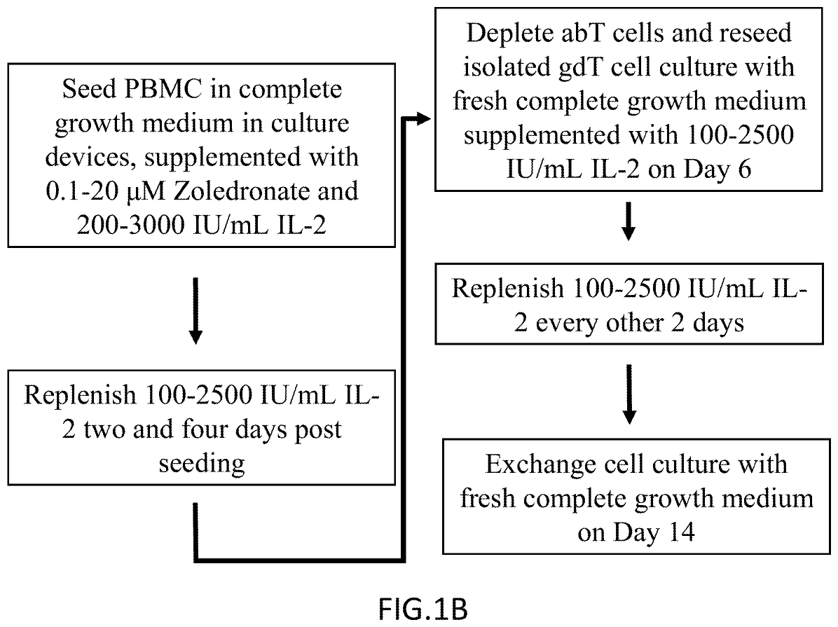

FIG. 1A provides exemplary procedures of methods described herein, including: (S11) in a device, culturing a cell population in a medium supplemented with a phosphoantigen, a first cytokine, and (iii) HPL; (S12) depleting the abT cells from the population of cells; and (S13) culturing the cell population for at least one day without phosphoantigen from the medium.

FIG. 1B also provides exemplary procedures of methods described herein, including: (1) Day 1: seed 5-200×10⁶ PBMCs in an air permeable culture device in complete growth medium supplemented with 0.1-20 μM zoledronate and 200-3000 IU/ml IL-2; (2) Day 2 and Day 4: replenish the culture medium with 100-2500 IU/ml IL-2; (3) Day 6: deplete abT cells and reseed remaining cells in complete growth medium supplemented with 100-2500 IU/ml IL-2; (4) Days 7-13: replenish the culture medium with 100-2500 IU/ml IL-2 every other day and reseed cells as needed; and (5) Day 14: change the culture medium to complete growth medium.

As a person of ordinary skill in the art would understand, a wide variety of combinations and permutations of various aspects of the methods disclosed herein exist. Such combinations and permutations are expressly contemplated as within the scope of this disclose.

### 5.2 Cell Populations Enriched in gdT Cells

Provided herein are also cell populations obtained by the methods described herein. The cell populations disclosed herein are enriched in gdT cells having NK-like properties, as indicated by the expression of certain biomarkers. In some embodiments, provided herein are vertebrate cell populations. In some embodiments, provided herein are mammalian cell populations. In some embodiments, the cell populations provided herein are human cell populations, non-human primate cell populations, canine cell populations, feline cell populations or rodent cell populations. In some embodiments, the cell populations provided herein are murine cell populations. In some embodiments, the cell populations provided herein are simian cell populations. In some embodiments, the cell populations provided herein are human cell populations.

Accordingly, in some embodiments, the gdT cells of the cell populations provided herein are vertebrate gdT cells. In some embodiments, the gdT cells are mammal gdT cells. In some embodiments, the gdT cells are selected from the group consisting of humans, non-human primates, canines, felines, rodents. In some embodiments, the gdT cells can be murine gdT cells. In some embodiments, the gdT cells can be simian gdT cells. In some embodiments, the gdT cells can be human gdT cells.

In some embodiments, the cell populations disclosed herein comprises 1×10⁶-1×10¹¹ cells, wherein 35-100% of the cells are gdT cells. In some embodiments, the cell populations disclosed herein comprise about 1×10⁶, about 1.5×10⁶, about 2×10⁶, about 2.5×10⁶, about 3×10⁶, about 3.5×10⁶, about 4×10⁶, about 4.5×10⁶, about 5×10⁶, about 5.5×10⁶, about 6×10⁶, about 6.5×10⁶, about 7×10⁶, about 7.5×10⁶, about 8×10⁶, about 8.5×10⁶, about 9×10⁶, about 9.5×10⁶, about 1×10⁷, about 1.5×10⁷, about 2×10⁷, about 2.5×10⁷, about 3×10⁷, about 3.5×10⁷, about 4×10⁷, about 4.5×10⁷, about 5×10⁷, about 5.5×10⁷, about 6×10⁷, about 6.5×10⁷, about 7×10⁷, about 7.5×10⁷, about 8×10⁷, about 8.5×10⁷, about 9×10⁷, about 9.5×10⁷, about 1×10⁸, about 1.5×10⁸, about 2×10⁸, about 2.5×10⁸, about 3×10⁸, about 3.5×10⁸, about 4×10⁸, about 4.5×10⁸, about 5×10⁸, about 5.5×10⁸, about 6×10⁸, about 6.5×10⁸, about 7×10⁸, about 7.5×10⁸, about 8×10⁸, about 8.5×10⁸, about 9×10⁸, about 9.5×10⁸, about 1×10⁹, about 1.5×10⁹, about 2×10⁹, about 2.5×10⁹, about 3×10⁹, about 3.5×10⁹, about 4×10⁹, about 4.5×10⁹, about 5×10⁹, about 5.5×10⁹, about 6×10⁹, about 6.5×10⁹, about 7×10⁹, about 7.5×10⁹, about 8×10⁹, about 8.5×10⁹, about 9×10⁹, about 9.5×10⁹, about 1×10¹⁰, about 1.5×10¹⁰, about 2×10¹⁰, about 2.5×10¹⁰, about 3×10¹⁰, about 3.5×10¹⁰, about 4×10¹⁰, about 4.5×10¹⁰, about 5×10¹⁰, about 5.5×10¹⁰, about 6×10¹⁰, about 6.5×10¹⁰, about 7×10¹⁰, about 7.5×10¹⁰, about 8×10¹⁰, about 8.5×10¹⁰, about 9×10¹⁰, about 9.5×10¹⁰, or about 1×10¹¹ cells, wherein 35-100% of the cells are gdT cells.

In some embodiments, the cell populations disclosed herein comprises about 1×10⁶ cells. The cell populations disclosed herein can comprise about 5×10⁶ cells. The cell populations disclosed herein can comprise about 1×10⁷ cells. The cell populations disclosed herein can comprise about 5×10⁷ cells. The cell populations disclosed herein can comprise about 1×10⁸ cells. The cell populations disclosed herein can comprise about 5×10⁸ cells. The cell populations disclosed herein can comprise about 1×10⁹ cells. The cell populations disclosed herein can comprise about 5×10⁹ cells. The cell populations disclosed herein can comprise about 1×10¹⁰ cells. The cell populations disclosed herein can comprise about 5×10¹⁰ cells. The cell populations disclosed herein can comprise about 1×10¹¹ cells.

The cell populations disclosed herein comprises 35-100% gdT cells. In some embodiments of the cell populations disclosed herein, at least 40%, at least 45%, at least 50%, at least 55%, at least 60%, at least 65%, at least 70%, at least 75%, at least 80%, at least 85%, at least 90%, or at least 90% of the cells are gdT cells. In some embodiments, at least 90%, at least 91%, at least 92%, at least 93%, at least 94%, at least 95%, at least 96%, at least 97%, at least 98%, or at least 99% of the cells are gdT cells. In some embodiments of the cell populations disclosed herein, at least 70% of the cells are gdT cells. In some embodiments, at least 75% of the cell population are gdT cells. In some embodiments, at least 80% of the cell population are gdT cells. In some embodiments, at least 85% of the cell population are gdT cells. In some embodiments, at least 90% of the cell population are gdT cells. In some embodiments, at least 95% of the cell population are gdT cells. In some embodiments, at least 98% of the cell population are gdT cells. In some embodiments, the cell populations provided herein have not been positively selected for gdT cells.

In some embodiments of the cell populations provided herein, no more than 30% of the cells are abT cells. In some embodiments, no more than 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1% of the cells in the in the cell populations described herein are abT cells. In some embodiments, the cell populations provided herein have no more than 5% abT cells. In some embodiments, the cell populations provided herein have no more than 2% abT cells. In some embodiments, the cell populations provided herein have no more than 1% abT cells. In some embodiments, the cell populations provided herein have no more than 0.5% abT cells. In some embodiments, the cell populations provided herein have no more than 0.1% abT cells. In some embodiments, the cell populations provided herein are substantially free of abT cells. In some embodiments, the cell populations provided herein do not have detectable abT cells.

In some embodiments, the cell populations disclosed herein comprise at least 0.5×10⁶, 1×10⁶, 2×10⁶, 3×10⁶, 4×10⁶, 5×10⁶, 5.5×10⁶, 6×10⁶, 6.5×10⁶, 7×10⁶, 7.5×10⁶, 8×10⁶, 8.5×10⁶, 9×10⁶, 9.5×10⁶, 1×10⁷, 1.5×10⁷, 2×10⁷, 2.5×10⁷, 3×10⁷, 3.5×10⁷, 4×10⁷, 4.5×10⁷, 5×10⁷, 5.5×10⁷, 6×10⁷, 6.5×10⁷, 7×10⁷, 7.5×10⁷, 8×10⁷, 8.5×10⁷, 9×10⁷, 9.5×10⁷, 1×10¹, 1.5×10⁸, 2×10⁸, 2.5×10⁸, 3×10⁸, 3.5×10⁸, 4×10⁸, 4.5×10⁸, 5×10⁸, 5.5×10⁸, 6×10⁸, 6.5×10⁸, 7×10⁸, 7.5×10⁸, 8×10⁸, 8.5×10⁸, 9×10⁸, 9.5×10⁸, 1×10⁹, 1.5×10⁹, 2×10⁹, 2.5×10⁹, 3×10⁹, 3.5×10⁹, 4×10⁹, 4.5×10⁹, 5×10⁹, 5.5×10⁹, 6×10⁹, 6.5×10⁹, 7×10⁹, 7.5×10⁹, 8×10⁹, 8.5×10⁹, 9×10⁹, 9.5×10⁹, 1×10¹⁰, 1.5×10¹⁰, 2×10¹⁰, 2.5×10¹⁰, 3×10¹⁰, 3.5×10¹⁰, 4×10¹⁰, 4.5×10¹⁰, 5×10¹⁰, 5.5×10¹⁰, 6×10¹⁰, 6.5×10¹⁰, 7×10¹⁰, 7.5×10¹⁰, 8×10¹⁰, 8.5×10¹⁰, 9×10¹⁰, 9.5×10¹⁰, or 1×10¹¹ cells gdT cells. In some embodiments, the cell populations disclosed herein comprise at least 1×10⁶, 5×10⁶, 1×10⁷, 5×10⁷, 1×10⁸, 5×10⁸, 1×10⁹, 5×10⁹, 1×10¹⁰, 5×10¹⁰, or 1×10¹¹ gdT cells. In some embodiments, the cell populations disclosed herein comprise at least 5×10⁶ gdT cells. In some embodiments, the cell populations comprise at least 1×10⁷ gdT cells. In some embodiments, the cell populations comprise at least 5×10⁷ gdT cells. In some embodiments, the cell populations comprise at least 1×10⁸ gdT cells. In some embodiments, the cell populations comprise at least 5×10⁸ gdT cells. In some embodiments, the cell populations comprise at least 1×10⁹ gdT cells. In some embodiments, the cell populations comprise at least 5×10⁹ gdT cells. In some embodiments, the cell populations comprise at least 1×10¹⁰ gdT cells. In some embodiments, the cell populations comprise at least 5×10¹⁰ gdT cells.

The gdT cells of the cell populations provided herein can comprise Vδ1 T cells, Vδ2 T cells, Vδ3 T cells, Vδ5 T cells, or any combination thereof. In some embodiments, at least 30% the gdT cells are Vδ2 T cells. In some embodiments, at least 30%, 35%, 40%, 45%, 50%, 55%, 60%, 65%, 70%, 75%, 80%, 85%, 90%, 91%, 92%, 93%, 94%, 95%, 96%, 97%, 98%, 99% or 100% of the gdT cells in the cell populations disclosed herein are Vδ2 T cell. In some embodiments, the gdT cells comprise Vγ9Vδ2 T cells. In some embodiments, at least 30%, 35%, 40%, 45%, 50%, 55%, 60%, 65%, 70%, 75%, 80%, 85%, 90%, 91%, 92%, 93%, 94%, 95%, 96%, 97%, 98%, 99% or 100% of the gdT cells in the cell populations disclosed herein are Vγ9Vδ2 T cell.

As known in the art, gdT cells can be further divided into the following four subsets of memory type: (1) terminally differentiated effector memory (TDEM or TEMRA) cells, characterized by CD45RA+CD27−; (2) central memory (CM or TCM) cells, characterized by CD45RA−CD27+; (3) naïve cells, characterized by CD45RA+CD27+; and (4) effector memory cells (EM or TEM) cells, characterized by CD45RA−CD27− (Guerra-Maupome et al., 2019, ImmunoHorizons. 3 (6) 208-218; Dieli et al., 2003, J Exp Med. 198(3):391-7). The cell populations enriched in gdT cells having NK-like properties are also characterized in that they comprise predominantly effector memory cells. In some embodiments of the cell populations provided herein, EM and TDEM cells constitute at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, or at least 98% of gdT cells of the cell populations provided herein. In some embodiments, EM and TDEM cells constitute at least 75% of gdT cells. In some embodiments, EM and TDEM cells constitute at least 80% of gdT cells. In some embodiments, EM and TDEM cells constitute at least 85% of gdT cells. In some embodiments, EM and TDEM cells constitute at least 90% of gdT cells. In some embodiments, EM and TDEM cells constitute at least 95% of gdT cells. In some embodiments, EM and TDEM cells constitute at least 98% of gdT cells.

In some embodiments, the cell populations provided herein comprise at least 10% TDEM cells. In some embodiments, the cell populations provided herein comprise 10-90% TDEM cells. In some embodiments, the cell populations provided herein comprise 10%, 15%, 20%, 25%, 30%, 35%, 40%, 45%, 50%, 55%, 60%, 65%, 70%, 75%, 80%, 85%, or 90% TDEM cells. In some embodiments, at least 30%, at least 40%, at least 50%, at least 60%, at least 70%, or at least 80% of the gdT cells are TDEM cells. In some embodiments, the cell populations provided herein comprise at least 30% TDEM cells. The cell populations provided herein can comprise at least 40% TDEM cells. The cell populations provided herein can comprise at least 50% TDEM cells. The cell populations provided herein can comprise at least 60% TDEM cells. The cell populations provided herein can comprise at least 70% TDEM cells. The cell populations provided herein can comprise at least 80% TDEM cells.

In some embodiments, the cell populations provided herein comprise at least 10% EM cells. In some embodiments, the cell populations provided herein comprise 10-90% EM cells. In some embodiments, the cell populations provided herein comprise at least 10%, 15%, 20%, 25%, 30%, 35%, 40%, 45%, 50%, 55%, 60%, 65%, 70%, 75%, 80%, 85%, or 90% EM cells.

In some embodiments, the cell populations provided herein comprise no more than 5% naïve cells. In some embodiments, the cell populations provided herein comprise no more than 1%, 2%, 3%, 4%, or 5% naïve cells. In some embodiments, the cell populations provided herein comprise 1-5% naïve cells.

In some embodiments, the cell populations provided herein comprise no more than 5% CM cells. In some embodiments, the cell populations provided herein comprise no more than 1%, 2%, 3%, 4%, or 5% central memory cells. In some embodiments, the cell populations provided herein comprise 1-5% CM cells.

CD69 expression represents activation in gdT cells. In some embodiments of the cell populations disclosed herein, at least 30% CD69+ cells. The cell populations disclosed herein can comprise at least 35%, at least 40%, at least 45%, at least 50%, at least 55%, at least 60%, at least 65%, at least 70%, at least 75%, at least 77%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% of the gdT cells are CD69+gdT cells. In some embodiments, at least 40%, at least 50%, at least 55%, at least 60%, at least 65%, at least 70%, at least 75%, or at least 80% of the gdT cells in the cell populations provided herein are CD69⁺. In some embodiments, at least 30% of the gdT cells in the cell populations provided herein are CD69+. In some embodiments, at least 35% of the gdT cells are CD69⁺. In some embodiments, at least 40% of the gdT cells are CD69⁺. In some embodiments, at least 45% of the gdT cells are CD69⁺. In some embodiments, at least 50% of the gdT cells are CD69⁺. In some embodiments, at least 55% of the gdT cells are CD69⁺. In some embodiments, at least 60% of the gdT cells are CD69⁺. In some embodiments, at least 65% of the gdT cells are CD69⁺. In some embodiments, at least 70% of the gdT cells are CD69⁺. In some embodiments, at least 75% of the gdT cells are CD69⁺. In some embodiments, at least 80% of the gdT cells are CD69⁺. In some embodiments, at least 85% of the gdT cells are CD69⁺. In some embodiments, at least 90% of the gdT cells are CD69⁺. In some embodiments, at least 95% of the gdT cells are CD69⁺. In some embodiments, at least 96% of the gdT cells are CD69⁺. In some embodiments, at least 97% of the gdT cells are CD69⁺. In some embodiments, at least 98% of the gdT cells are CD69⁺.

In some embodiments, the cell populations provided herein comprise at least 5×10⁵, 1×10⁶, 2×10⁶, 3×10⁶, 4×10⁶, 5×10⁶, 5.5×10⁶, 6×10⁶, 6.5×10⁶, 7×10⁶, 7.5×10⁶, 8×10⁶, 8.5×10⁶, 9×10⁶, 9.5×10⁶, 1×10⁷, 1.5×10⁷, 2×10⁷, 2.5×10⁷, 3×10⁷, 3.5×10⁷, 4×10⁷, 4.5×10⁷, 5×10⁷, 5.5×10⁷, 6×10⁷, 6.5×10⁷, 7×10⁷, 7.5×10⁷, 8×10⁷, 8.5×10⁷, 9×10⁷, 9.5×10⁷, 1×10⁸, 1.5×10⁸, 2×10⁸, 2.5×10⁸, 3×10⁸, 3.5×10⁸, 4×10⁸, 4.5×10⁸, 5×10⁸, 5.5×10⁸, 6×10⁸, 6.5×10⁸, 7×10⁸, 7.5×10⁸, 8×10⁸, 8.5×10⁸, 9×10⁸, 9.5×10, 1×10⁹, 1.5×10⁹, 2×10⁹, 2.5×10⁹, 3×10⁹, 3.5×10, 4×10⁹, 4.5×10, 5×10⁹, 5.5×10⁹, 6×10⁹, 6.5×10⁹, 7×10⁹, 7.5×10⁹, 8×10⁹, 8.5×10⁹, 9×10⁹, 9.5×10⁹, 1×10¹⁰, 1.5×10¹⁰, 2×10¹⁰, 2.5×10¹⁰, 3×10¹⁰, 3.5×10¹⁰, 4×10¹⁰, 4.5×10¹⁰, 5×10¹⁰, 5.5×10¹⁰, 6×10¹⁰, 6.5×10¹⁰, 7×10¹⁰, 7.5×10¹⁰, 8×10¹⁰, 8.5×10¹⁰, 9×10¹⁰, 9.5×10¹⁰, or 1×10¹¹ cells CD69+gdT cells. In some embodiments, the cell populations disclosed herein comprise at least 1×10⁶ CD69+gdT cells. In some embodiments, the cell populations comprise at least 5×10⁶ CD69+gdT cells. In some embodiments, the cell populations comprise at least 1×10⁷ CD69+gdT cells. In some embodiments, the cell populations comprise at least 5×10⁷ CD69+gdT cells. In some embodiments, the cell populations comprise at least 1×10⁸ CD69+gdT cells. In some embodiments, the cell populations comprise at least 5×10⁸ CD69+gdT cells. In some embodiments, the cell populations comprise at least 1×10⁹ CD69+gdT cells. In some embodiments, the cell populations comprise at least 5×10⁹ CD69+gdT cells. In some embodiments, the cell populations comprise at least 1×10¹⁰ CD69+gdT cells. In some embodiments, the cell populations comprise at least 5×10¹⁰ CD69+gdT cells.

In some embodiments of the cell populations provided herein, the gdT cells express at least 400 CD69 molecules per cell on average. In some embodiments, the gdT cells express at least 500, 600, 700, 800, 900, 1000, 1500, 2000, 2500, 3000, 3500, 4000, 4500, 5000, 5500, 6000, 6500, 7000, 7500, 8000, 8500, 9000, 9500, 10000, 15000, 20000, 25000, 30000, 35000, 40000, 45000, 50000, 55000, 60000, 65000, 70000, 75000, 80000, 85000, 90000, 95000, 100000, 110000, 120000, 130000, 140000, 150000, 160000, 170000, 180000, 190000, 200000, 210000, 220000, 230000, 240000, 250000, 260000, 270000, 280000, 290000, 300000, 310000, 320000, 330000, 340000, 350000, 360000, 370000, 380000, 390000, 400000, 410000, 420000, 430000, 440000, 450000, 460000, 470000, 480000, 490000, or 500000 CD69 molecules per cell on average. In some embodiments, the gdT cells express at least 5000 CD69 molecules per cell on average. The gdT cells can express about 5000 to about 70000 CD69 molecules per cell on average. In some embodiments, the gdT cells express at least 10000 CD69 molecules per cell on average. The gdT cells can express about 10000 to about 70000 CD69 molecules per cell on average. In some embodiments, the gdT cells express at least 20000 CD69 molecules per cell on average. The gdT cells can express about 20000 to about 70000 CD69 molecules per cell on average. In some embodiments, the gdT cells express at least 30000 CD69 molecules per cell on average. The gdT cells can express about 30000 to about 70000 CD69 molecules per cell on average. In some embodiments, the gdT cells express at least 40000 CD69 molecules per cell on average. The gdT cells can express about 40000 to about 70000 CD69 molecules per cell on average. In some embodiments, the gdT cells express at least 50000 CD69 molecules per cell on average. The gdT cells can express about 50000 to about 70000 CD69 molecules per cell on average. In some embodiments, the gdT cells express at least 60000 CD69 molecules per cell on average. The gdT cells can express about 60000 to about 70000 CD69 molecules per cell on average. In some embodiments, the gdT cells express at least 70000 CD69 molecules per cell on average. The gdT cells can express about 70000 to about 100000 CD69 molecules per cell on average.

In some embodiments of the cell populations disclosed herein, the CD69-expressing gdT cells express at least 400 CD69 molecules per cell on average. In some embodiments, the CD69-expressing gdT cells express at least 500, 600, 700, 800, 900, 1000, 1500, 2000, 2500, 3000, 3500, 4000, 4500, 5000, 5500, 6000, 6500, 7000, 7500, 8000, 8500, 9000, 9500, 10000, 15000, 20000, 25000, 30000, 35000, 40000, 45000, 50000, 55000, 60000, 65000, 70000, 75000, 80000, 85000, 90000, 95000, 100000, 110000, 120000, 130000, 140000, 150000, 160000, 170000, 180000, 190000, 200000, 210000, 220000, 230000, 240000, 250000, 260000, 270000, 280000, 290000, 300000, 310000, 320000, 330000, 340000, 350000, 360000, 370000, 380000, 390000, 400000, 410000, 420000, 430000, 440000, 450000, 460000, 470000, 480000, 490000, or 500000 CD69 molecules per cell on average. In some embodiments, the CD69-expressing gdT cells express at least 5000 CD69 molecules per cell on average. The CD69-expressing gdT cells can express about 5000 to about 70000 CD69 molecules per cell on average. In some embodiments, the CD69-expressing gdT cells express at least 10000 CD69 molecules per cell on average. The CD69-expressing gdT cells can express about 10000 to about 70000 CD69 molecules per cell on average. In some embodiments, the CD69-expressing gdT cells express at least 20000 CD69 molecules per cell on average. The CD69-expressing gdT cells can express about 20000 to about 70000 CD69 molecules per cell on average. In some embodiments, the CD69-expressing gdT cells express at least 30000 CD69 molecules per cell on average. The CD69-expressing gdT cells can express about 30000 to about 70000 CD69 molecules per cell on average. In some embodiments, the CD69-expressing gdT cells express at least 40000 CD69 molecules per cell on average. The CD69-expressing gdT cells can express about 40000 to about 70000 CD69 molecules per cell on average. In some embodiments, the CD69-expressing gdT cells express at least 50000 CD69 molecules per cell on average. The CD69-expressing gdT cells can express about 50000 to about 70000 CD69 molecules per cell on average. In some embodiments, the CD69-expressing gdT cells express at least 60000 CD69 molecules per cell on average. The CD69-expressing gdT cells can express about 60000 to about 70000 CD69 molecules per cell on average. In some embodiments, the CD69-expressing gdT cells express at least 70000 CD69 molecules per cell on average. The CD69-expressing gdT cells can express about 70000 to about 100000 CD69 molecules per cell on average.

In some embodiments of the cell populations provided herein: CD69-expressing gdT cells each expresses at least 400, 500, 600, 700, 800, 900, 1000, 1500, 2000, 2500, 3000, 3500, 4000, 4500, 5000, 5500, 6000, 6500, 7000, 7500, 8000, 8500, 9000, 9500, 10000, 15000, 20000, 25000, 30000, 35000, 40000, 45000, 50000, 55000, 60000, 65000, 70000, 75000, 80000, 85000, 90000, 95000, 100000, 110000, 120000, 130000, 140000, 150000, 160000, 170000, 180000, 190000, 200000, 210000, 220000, 230000, 240000, 250000, 260000, 270000, 280000, 290000, 300000, 310000, 320000, 330000, 340000, 350000, 360000, 370000, 380000, 390000, 400000, 410000, 420000, 430000, 440000, 450000, 460000, 470000, 480000, 490000, or 500000 CD69 molecules.

In some embodiments of the cell populations provided herein, 30-100% of the gdT cells express DNAM-1. In some embodiments, at least 30%, 35%, 40%, 45%, 50%, 55%, 60%, 65%, 70%, 75%, 80%, 85%, 90%, 91%, 92%, 93%, 94%, 95%, 96%, 97%, 98%, or 99% of the gdT cells express DNAM-1. In some embodiments, at least 50% of the cells express DNAM-1. In some embodiments, at least 60% of the cells express DNAM-1. In some embodiments, at least 70% of the cells express DNAM-1. In some embodiments, at least 80% of the cells express DNAM-1. In some embodiments, at least 90% of the cells express DNAM-1.

In some embodiments of the cell populations provided herein, the gdT cells express at least 300, 400, 500, 600, 700, 800, 900, 1000, 2000, 3000, 4000, 5000, 5500, 6000, 6500, 7000, 7500, 8000, 8500, 9000, 9500, 10000, 15000, 20000, 25000, 30000, 35000, 40000, 45000, 50000, 55000, 60000, 65000, 70000, 75000, 80000, 85000, 90000, 95000, 100000, 110000, 120000, 130000, 140000, 150000, 160000, 170000, 180000, 190000, 200000, 210000, 220000, 230000, 240000, 250000, 260000, 270000, 280000, 290000, or 300000 DNAM-1 molecules per cell on average. In some embodiments, the gdT cells express at least 400 DNAM-1 molecules per cell on average. The gdT cells can express about 400 to about 300000 DNAM-1 molecules per cell on average. In some embodiments, the gdT cells express at least 500, at least 600, at least 700, at least 800, at least 900, at least 1000, at least 2000, or at least 3000 DNAM-1 molecules per cell on average. In some embodiments, the gdT cells express at least 1000 DNAM-1 molecules per cell on average. The gdT cells can express about 1000 to about 300000 DNAM-1 molecules per cell on average. In some embodiments, the gdT cells express at least 5000 DNAM-1 molecules per cell on average. In some embodiments, the gdT cells express at least 10000 DNAM-1 molecules per cell on average. The gdT cells can express about 10000 to about 300000 DNAM-1 molecules per cell on average. In some embodiments, the gdT cells express at least 20000 DNAM-1 molecules per cell on average. In some embodiments, the gdT cells express at least 50000 DNAM-1 molecules per cell on average. The gdT cells can express about 50000 to about 300000 DNAM-1 molecules per cell on average. In some embodiments, the gdT cells express at least 80000 DNAM-1 molecules per cell on average. In some embodiments, the gdT cells express at least 100000 DNAM-1 molecules per cell on average. The gdT cells can express about 100000 to about 300000 DNAM-1 molecules per cell on average. In some embodiments, the gdT cells express at least 150000 DNAM-1 molecules per cell on average. In some embodiments, the gdT cells express at least 200000 DNAM-1 molecules per cell on average. The gdT cells can express about 200000 to about 300000 DNAM-1 molecules per cell on average.

In some embodiments of the cell populations provided herein, the DNAM-1-expressing gdT cells express at least 300, 400, 500, 1000, 2000, 3000, 4000, 5000, 5500, 6000, 6500, 7000, 7500, 8000, 8500, 9000, 9500, 10000, 15000, 20000, 25000, 30000, 35000, 40000, 45000, 50000, 55000, 60000, 65000, 70000, 75000, 80000, 85000, 90000, 95000, 100000, 110000, 120000, 130000, 140000, 150000, 160000, 170000, 180000, 190000, 200000, or 210000, 220000, 230000, 240000, 250000, 260000, 270000, 280000, 290000, or 300000 DNAM-1 molecules per cell on average. In some embodiments, 30-100% of the gdT cells in the composition express 500-300000 DNAM-1 molecules per cell on average. In some embodiments, the DNAM-1-expressing gdT cells express at least 400 DNAM-1 molecules per cell on average. The DNAM-1-expressing gdT cells can express about 400 to about 300000 DNAM-1 molecules per cell on average. In some embodiments, the DNAM-1-expressing gdT cells express at least 1000 DNAM-1 molecules per cell on average. The DNAM-1-expressing gdT cells can express about 1000 to about 300000 DNAM-1 molecules per cell on average. In some embodiments, the DNAM-1-expressing gdT cells express at least 5000 DNAM-1 molecules per cell on average. In some embodiments, the DNAM-1-expressing gdT cells express at least 10000 DNAM-1 molecules per cell on average. The DNAM-1-expressing gdT cells can express about 10000 to about 300000 DNAM-1 molecules per cell on average. In some embodiments, the DNAM-1-expressing gdT cells express at least 20000 DNAM-1 molecules per cell on average. In some embodiments, the DNAM-1-expressing gdT cells express at least 50000 DNAM-1 molecules per cell on average. The DNAM-1-expressing gdT cells can express about 50000 to about 300000 DNAM-1 molecules per cell on average. In some embodiments, the DNAM-1-expressing gdT cells express at least 80000 DNAM-1 molecules per cell on average. In some embodiments, the DNAM-1-expressing gdT cells express at least 100000 DNAM-1 molecules per cell on average. The DNAM-1-expressing gdT cells can express about 100000 to about 300000 DNAM-1 molecules per cell on average. In some embodiments, the DNAM-1-expressing gdT cells express at least 150000 DNAM-1 molecules per cell on average. In some embodiments, the DNAM-1-expressing gdT cells express at least 200000 DNAM-1 molecules per cell on average. The DNAM-1-expressing gdT cells can express about 200000 to about 300000 DNAM-1 molecules per cell on average.

In some embodiments of the cell populations provided herein, the DNAM-1-expressing gdT cells each express at least 300, 400, 500, 600, 700, 800, 900, 1000, 2000, 3000, 4000, 5000, 5500, 6000, 6500, 7000, 7500, 8000, 8500, 9000, 9500, 10000, 15000, 20000, 25000, 30000, 35000, 40000, 45000, 50000, 55000, 60000, 65000, 70000, 75000, 80000, 85000, 90000, 95000, 100000, 110000, 120000, 130000, 140000, 150000, 160000, 170000, 180000, 190000, 200000, 210000, 220000, 230000, 240000, 250000, 260000, 270000, 280000, 290000, or 300000 DNAM-1 molecules.

The anti-tumor activity of the cell populations described herein is also reflected in the enhanced expression of NK cytotoxicity receptors (e.g., CD56, CD16, NKG2D, NKp44, and NKp46) and degranulation markers (e.g., CD107a). In some embodiments of the cell populations provided herein, an increased percentage of gdT cells express (1) cytotoxicity receptors (e.g., CD56, CD16, NKG2D, NKp44 and NKp46), and/or (2) degranulation markers (e.g., CD107a). In some embodiments of the cell populations provided herein, the gdT cells expressing (1) cytotoxicity receptors (e.g., CD56, CD16, NKG2D, NKp44 and NKp46) and/or (2) degranulation markers (e.g., CD107a) express more molecules per cell on average (i.e., having a higher Number of Molecules per Cell, or “NMC”). The cell populations provided herein can be further characterized in the enhanced expression of additional markers that indicate the therapeutic potential of the gdT cells, including, for example, INFγ, DNAM-1, Granzyme B, TIGIT, CD18, NKp30, CCR7, CD25, CD38, CD36, and CD103, as well as in the decreased expression of marker that indicates the lack of activity of the gdT cells, such as PD-1.

In some embodiments of the cell populations provided herein, at least 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% of the gdT cells express CD56. In some embodiments, at least 30% of the gdT cells express CD56. In some embodiments, at least 40% of the gdT cells express CD56. In some embodiments, at least 50% of the gdT cells express CD56. In some embodiments, at least 60% of the gdT cells express CD56. In some embodiments, at least 70% of the gdT cells express CD56. In some embodiments, about 30% to about 80% of the gdT cells express CD56. In some embodiments, about 40% to about 80% of the gdT cells express CD56. In some embodiments, about 50% to about 80% of the gdT cells express CD56. In some embodiments, about 60% to about 80% of the gdT cells express CD56.

In some embodiments of the cell populations provided herein, the gdT cells express at least 400, 500, 600, 700, 800, 900, 1000, 1500, 2000, 2500, 3000, 3500, 4000, 4500, 5000, 5500, 6000, 6500, 7000, 7500, 8000, 8500, 9000, 9500, 10000, 15000, 20000, 25000, 30000, 35000, 40000, 45000, 50000, 55000, 60000, 65000, 70000, 75000, 80000, 85000, 90000, 95000, 100000, 110000, 120000, 130000, 140000, 150000, 160000, 170000, 180000, 190000, or 200000, 210000, 220000, 230000, 240000, 250000, 260000, 270000, 280000, 290000, 300000, 310000, 320000, 330000, 340000, 350000, 360000, 370000, 380000, 390000, 400000, 410000, 420000, 430000, 440000, 450000, 460000, 470000, 480000, 490000, or 500000 CD56 molecules per cell on average. In some embodiments, the gdT cells provided herein express at least 400 CD56 molecules per cell on average. In some embodiments, the gdT cells provided herein express at least 1000 CD56 molecules per cell on average. The gdT cells provided herein can express about 1000 to about 80000 CD56 molecules per cell on average. In some embodiments, the gdT cells provided herein express at least 5000 CD56 molecules per cell on average. The gdT cells provided herein can express about 5000 to about 80000 CD56 molecules per cell on average. In some embodiments, the gdT cells provided herein express at least 10000 CD56 molecules per cell on average. The gdT cells provided herein can express about 10000 to about 80000 CD56 molecules per cell on average. In some embodiments, the gdT cells provided herein express at least 20000 CD56 molecules per cell on average. In some embodiments, the gdT cells provided herein express at least 30000 CD56 molecules per cell on average. In some embodiments, the gdT cells provided herein express at least 50000 CD56 molecules per cell on average. The gdT cells provided herein can express about 50000 to about 80000 CD56 molecules per cell on average. In some embodiments, the gdT cells provided herein express at least 60000 CD56 molecules per cell on average. The gdT cells provided herein can express about 60000 to about 80000 CD56 molecules per cell on average. In some embodiments, the gdT cells provided herein express at least 70000 CD56 molecules per cell on average. The gdT cells provided herein can express about 70000 to about 100000 CD56 molecules per cell on average.

In some embodiments of the cell populations provided herein, at least 10%, 15%, 20%, 25%, 30%, 35%, 40%, 45%, 50%, 55%, 60%, 65%, 70%, 75%, or 80% of the gdT cells express CD56, wherein the CD56-expressing gdT cells express at least 400, 500, 600, 700, 800, 900, 1000, 1500, 2000, 2500, 3000, 3500, 4000, 4500, 5000, 5500, 6000, 6500, 7000, 7500, 8000, 8500, 9000, 9500, 10000, 15000, 20000, 25000, 30000, 35000, 40000, 45000, 50000, 55000, 60000, 65000, 70000, 75000, 80000, 85000, 90000, 95000, 100000, 110000, 120000, 130000, 140000, 150000, 160000, 170000, 180000, 190000, 200000, 210000, 220000, 230000, 240000, 250000, 260000, 270000, 280000, 290000, 300000, 310000, 320000, 330000, 340000, 350000, 360000, 370000, 380000, 390000, 400000, 410000, 420000, 430000, 440000, 450000, 460000, 470000, 480000, 490000, or 500000 CD56 molecules per cell on average. In some embodiments, the CD56-expressing gdT cells provided herein express at least 1000 CD56 molecules per cell on average. The CD56-expressing gdT cells provided herein can express about 1000 to about 80000 CD56 molecules per cell on average. In some embodiments, the CD56-expressing gdT cells provided herein express at least 5000 CD56 molecules per cell on average. The CD56-expressing gdT cells provided herein can express about 5000 to about 80000 CD56 molecules per cell on average. In some embodiments, the CD56-expressing gdT cells provided herein express at least 10000 CD56 molecules per cell on average. The CD56-expressing gdT cells provided herein can express about 10000 to about 80000 CD56 molecules per cell on average. In some embodiments, the CD56-expressing gdT cells provided herein express at least 20000 CD56 molecules per cell on average. In some embodiments, the CD56-expressing gdT cells provided herein express at least 30000 CD56 molecules per cell on average. In some embodiments, the CD56-expressing gdT cells provided herein express at least 50000 CD56 molecules per cell on average. The CD56-expressing gdT cells provided herein can express about 50000 to about 80000 CD56 molecules per cell on average. In some embodiments, the CD56-expressing gdT cells provided herein express at least 60000 CD56 molecules per cell on average. The CD56-expressing gdT cells provided herein can express about 60000 to about 80000 CD56 molecules per cell on average. In some embodiments, the CD56-expressing gdT cells provided herein express at least 70000 CD56 molecules per cell on average. The CD56-expressing gdT cells provided herein can express about 70000 to about 100000 CD56 molecules per cell on average.

In some embodiments of the cell populations provided herein, the CD56-expressing gdT cells each expresses at least 400, 500, 600, 700, 800, 900, 1000, 1500, 2000, 2500, 3000, 3500, 4000, 4500, 5000, 5500, 6000, 6500, 7000, 7500, 8000, 8500, 9000, 9500, 10000, 15000, 20000, 25000, 30000, 35000, 40000, 45000, 50000, 55000, 60000, 65000, 70000, 75000, 80000, 85000, 90000, 95000, 100000, 110000, 120000, 130000, 140000, 150000, 160000, 170000, 180000, 190000, or 200000, 210000, 220000, 230000, 240000, 250000, 260000, 270000, 280000, 290000, 300000, 310000, 320000, 330000, 340000, 350000, 360000, 370000, 380000, 390000, 400000, 410000, 420000, 430000, 440000, 450000, 460000, 470000, 480000, 490000, or 500000 CD56 molecules.

In some embodiments of the cell populations provided herein, at least 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% of the gdT cells express CD16. In some embodiments, at least 20% of the gdT cells express CD16. In some embodiments, at least 30% of the gdT cells express CD16. In some embodiments, at least 40% of the gdT cells express CD16. In some embodiments, at least 50% of the gdT cells express CD16. In some embodiments, at least 60% of the gdT cells express CD16. In some embodiments, at least 70% of the gdT cells express CD16. In some embodiments, at least 80% of the gdT cells express CD16. In some embodiments, about 20% to about 90% of the gdT cells express CD16. In some embodiments, about 30% to about 90% of the gdT cells express CD16. In some embodiments, about 40% to about 90% of the gdT cells express CD16. In some embodiments, about 60% to about 90% of the gdT cells express CD16.

In some embodiments of the cell populations provided herein, the gdT cells express at least 400, 500, 600, 700, 800, 900, 1000, 1500, 2000, 2500, 3000, 3500, 4000, 4500, 5000, 5500, 6000, 6500, 7000, 7500, 8000, 8500, 9000, 9500, 10000, 15000, 20000, 25000, 30000, 35000, 40000, 45000, 50000, 55000, 60000, 65000, 70000, 75000, 80000, 85000, 90000, 95000, 100000, 110000, 120000, 130000, 140000, 150000, 160000, 170000, 180000, 190000, 200000, 210000, 220000, 230000, 240000, 250000, 260000, 270000, 280000, 290000, 300000, 310000, 320000, 330000, 340000, 350000, 360000, 370000, 380000, 390000, 400000, 410000, 420000, 430000, 440000, 450000, 460000, 470000, 480000, 490000, or 500000 CD16 molecules per cell on average. In some embodiments, 10%-100% of the gdT cells in the composition express 400-500000 CD16 molecules per cell on average. In some embodiments, the gdT cells provided herein express at least 400 CD16 molecules per cell on average. In some embodiments, the gdT cells provided herein express at least 1000 CD16 molecules per cell on average. The gdT cells provided herein can express about 1000 to about 90000 CD16 molecules per cell on average. In some embodiments, the gdT cells provided herein express at least 5000 CD16 molecules per cell on average. The gdT cells provided herein can express about 5000 to about 90000 CD16 molecules per cell on average. In some embodiments, the gdT cells provided herein express at least 10000 CD16 molecules per cell on average. The gdT cells provided herein can express about 10000 to about 90000 CD16 molecules per cell on average. In some embodiments, the gdT cells provided herein express at least 20000 CD16 molecules per cell on average. In some embodiments, the gdT cells provided herein express at least 30000 CD16 molecules per cell on average. In some embodiments, the gdT cells provided herein express at least 50000 CD16 molecules per cell on average. The gdT cells provided herein can express about 50000 to about 90000 CD16 molecules per cell on average. In some embodiments, the gdT cells provided herein express at least 60000 CD16 molecules per cell on average. The gdT cells provided herein can express about 60000 to about 90000 CD16 molecules per cell on average. In some embodiments, the gdT cells provided herein express at least 70000 CD16 molecules per cell on average. In some embodiments, the gdT cells provided herein express at least 80000 CD16 molecules per cell on average. The gdT cells provided herein can express about 70000 to about 100000 CD16 molecules per cell on average. The gdT cells provided herein can express about 70000 to about 90000 CD16 molecules per cell on average. The gdT cells provided herein can express about 80000 to about 90000 CD16 molecules per cell on average.

In some embodiments of the cell populations provided herein, the CD16-expressing gdT cells express at least 400, 500, 600, 700, 800, 900, 1000, 1500, 2000, 2500, 3000, 3500, 4000, 4500, 5000, 5500, 6000, 6500, 7000, 7500, 8000, 8500, 9000, 9500, 10000, 15000, 20000, 25000, 30000, 35000, 40000, 45000, 50000, 55000, 60000, 65000, 70000, 75000, 80000, 85000, 90000, 95000, 100000, 110000, 120000, 130000, 140000, 150000, 160000, 170000, 180000, 190000, 200000, 210000, 220000, 230000, 240000, 250000, 260000, 270000, 280000, 290000, 300000, 310000, 320000, 330000, 340000, 350000, 360000, 370000, 380000, 390000, 400000, 410000, 420000, 430000, 440000, 450000, 460000, 470000, 480000, 490000, or 500000 CD16 molecules per cell on average. In some embodiments, the CD16-expressing gdT cells provided herein express at least 400 CD16 molecules per cell on average. In some embodiments, the CD16-expressing gdT cells provided herein express at least 1000 CD16 molecules per cell on average. The CD16-expressing gdT cells provided herein can express about 1000 to about 90000 CD16 molecules per cell on average. In some embodiments, the CD16-expressing gdT cells provided herein express at least 5000 CD16 molecules per cell on average. The CD16-expressing gdT cells provided herein can express about 5000 to about 90000 CD16 molecules per cell on average. In some embodiments, the CD16-expressing gdT cells provided herein express at least 10000 CD16 molecules per cell on average. The CD16-expressing gdT cells provided herein can express about 10000 to about 90000 CD16 molecules per cell on average. In some embodiments, the CD16-expressing gdT cells provided herein express at least 20000 CD16 molecules per cell on average. In some embodiments, the CD16-expressing gdT cells provided herein express at least 30000 CD16 molecules per cell on average. In some embodiments, the CD16-expressing gdT cells provided herein express at least 50000 CD16 molecules per cell on average. The CD16-expressing gdT cells provided herein can express about 50000 to about 90000 CD16 molecules per cell on average. In some embodiments, the CD16-expressing gdT cells provided herein express at least 60000 CD16 molecules per cell on average. The CD16-expressing gdT cells provided herein can express about 60000 to about 90000 CD16 molecules per cell on average. In some embodiments, the CD16-expressing gdT cells provided herein express at least 70000 CD16 molecules per cell on average. In some embodiments, the CD16-expressing gdT cells provided herein express at least 80000 CD16 molecules per cell on average. The CD16-expressing gdT cells provided herein can express about 70000 to about 100000 CD16 molecules per cell on average. The CD16-expressing gdT cells provided herein can express about 70000 to about 90000 CD16 molecules per cell on average. The CD16-expressing gdT cells provided herein can express about 80000 to about 90000 CD16 molecules per cell on average.

In some embodiments of the cell populations provided herein, the CD16-expressing gdT cells each expresses at least 400, 500, 600, 700, 800, 900, 1000, 1500, 2000, 2500, 3000, 3500, 4000, 4500, 5000, 5500, 6000, 6500, 7000, 7500, 8000, 8500, 9000, 9500, 10000, 15000, 20000, 25000, 30000, 35000, 40000, 45000, 50000, 55000, 60000, 65000, 70000, 75000, 80000, 85000, 90000, 95000, 100000, 110000, 120000, 130000, 140000, 150000, 160000, 170000, 180000, 190000, 200000, 210000, 220000, 230000, 240000, 250000, 260000, 270000, 280000, 290000, 300000, 310000, 320000, 330000, 340000, 350000, 360000, 370000, 380000, 390000, 400000, 410000, 420000, 430000, 440000, 450000, 460000, 470000, 480000, 490000, or 500000 CD16 molecules.

In some embodiments of the cell populations provided herein, at least 30%, 35%, 40%, 45%, 50%, 55%, 60%, 65%, 70%, 75%, 80%, 85%, 90%, 91%, 92%, 93%, 94%, 95%, 96%, 97%, 98%, or 99% of the gdT cells express NKG2D. In some embodiments, at least 30% of the gdT cells express NKG2D. In some embodiments, at least 40% of the gdT cells express NKG2D. In some embodiments, at least 50% of the gdT cells express NKG2D. In some embodiments, at least 60% of the gdT cells express NKG2D. In some embodiments, at least 70% of the gdT cells express NKG2D. In some embodiments, at least 80% of the gdT cells express NKG2D. In some embodiments, at least 90% of the gdT cells express NKG2D.

In some embodiments of the cell populations provided herein, the gdT cells express at least 400, 500, 600, 700, 800, 900, 1000, 1500, 2000, 2500, 3000, 3500, 4000, 4500, 5000, 5500, 6000, 6500, 7000, 7500, 8000, 8500, 9000, 9500, 10000, 15000, 20000, 25000, 30000, 35000, 40000, 45000, 50000, 55000, 60000, 65000, 70000, 75000, 80000, 85000, 90000, 95000, 100000, 110000, 120000, 130000, 140000, 150000, 160000, 170000, 180000, 190000, 200000, 210000, 220000, 230000, 240000, 250000, 260000, 270000, 280000, 290000, 300000, 310000, 320000, 330000, 340000, 350000, 360000, 370000, 380000, 390000, 400000, 410000, 420000, 430000, 440000, 450000, 460000, 470000, 480000, 490000, or 500000 NKG2D molecules per cell on average. In some embodiments, the gdT cells provided herein express at least 400 NKG2D molecules per cell on average. In some embodiments, the gdT cells provided herein express at least 1000 NKG2D molecules per cell on average. The gdT cells provided herein can express about 1000 to about 80000 NKG2D molecules per cell on average. In some embodiments, the gdT cells provided herein express at least 5000 NKG2D molecules per cell on average. The gdT cells provided herein can express about 5000 to about 80000 NKG2D molecules per cell on average. In some embodiments, the gdT cells provided herein express at least 10000 NKG2D molecules per cell on average. The gdT cells provided herein can express about 10000 to about 80000 NKG2D molecules per cell on average. In some embodiments, the gdT cells provided herein express at least 20000 NKG2D molecules per cell on average. In some embodiments, the gdT cells provided herein express at least 30000 NKG2D molecules per cell on average. In some embodiments, the gdT cells provided herein express at least 50000 NKG2D molecules per cell on average. The gdT cells provided herein can express about 50000 to about 80000 NKG2D molecules per cell on average. In some embodiments, the gdT cells provided herein express at least 60000 NKG2D molecules per cell on average. The gdT cells provided herein can express about 60000 to about 80000 NKG2D molecules per cell on average. In some embodiments, the gdT cells provided herein express at least 70000 NKG2D molecules per cell on average. The gdT cells provided herein can express about 70000 to about 100000 NKG2D molecules per cell on average. The gdT cells provided herein can express about 70000 to about 80000 NKG2D molecules per cell on average.

In some embodiments, 30-100% of the gdT cells in the composition express at least 40-500000 NKG2D molecules per cell on average. In some embodiments of the cell populations provided herein, the NKG2D-expressing gdT cells express at least 400, 500, 600, 700, 800, 900, 1000, 1500, 2000, 2500, 3000, 3500, 4000, 4500, 5000, 5500, 6000, 6500, 7000, 7500, 8000, 8500, 9000, 9500, 10000, 15000, 20000, 25000, 30000, 35000, 40000, 45000, 50000, 55000, 60000, 65000, 70000, 75000, 80000, 85000, 90000, 95000, 100000, 110000, 120000, 130000, 140000, 150000, 160000, 170000, 180000, 190000, 200000, 210000, 220000, 230000, 240000, 250000, 260000, 270000, 280000, 290000, 300000, 310000, 320000, 330000, 340000, 350000, 360000, 370000, 380000, 390000, 400000, 410000, 420000, 430000, 440000, 450000, 460000, 470000, 480000, 490000, or 500000 NKG2D molecules per cell on average. In some embodiments, the NKG2D-expressing gdT cells provided herein express at least 400 NKG2D molecules per cell on average. In some embodiments, the NKG2D-expressing gdT cells provided herein express at least 1000 NKG2D molecules per cell on average. The NKG2D-expressing gdT cells provided herein can express about 1000 to about 80000 NKG2D molecules per cell on average. In some embodiments, the NKG2D-expressing gdT cells provided herein express at least 5000 NKG2D molecules per cell on average. The NKG2D-expressing gdT cells provided herein can express about 5000 to about 80000 NKG2D molecules per cell on average. In some embodiments, the NKG2D-expressing gdT cells provided herein express at least 10000 NKG2D molecules per cell on average. The NKG2D-expressing gdT cells provided herein can express about 10000 to about 80000 NKG2D molecules per cell on average. In some embodiments, the NKG2D-expressing gdT cells provided herein express at least 20000 NKG2D molecules per cell on average. In some embodiments, the NKG2D-expressing gdT cells provided herein express at least 30000 NKG2D molecules per cell on average. In some embodiments, the NKG2D-expressing gdT cells provided herein express at least 50000 NKG2D molecules per cell on average. The NKG2D-expressing gdT cells provided herein can express about 50000 to about 80000 NKG2D molecules per cell on average. In some embodiments, the NKG2D-expressing gdT cells provided herein express at least 60000 NKG2D molecules per cell on average. The NKG2D-expressing gdT cells provided herein can express about 60000 to about 80000 NKG2D molecules per cell on average. In some embodiments, the NKG2D-expressing gdT cells provided herein express at least 70000 NKG2D molecules per cell on average. The NKG2D-expressing gdT cells provided herein can express about 70000 to about 100000 NKG2D molecules per cell on average. The NKG2D-expressing gdT cells provided herein can express about 70000 to about 80000 NKG2D molecules per cell on average.

In some embodiments of the cell populations provided herein, the NKG2D-expressing gdT cells each expresses at least 400, 500, 600, 700, 800, 900, 1000, 1500, 2000, 2500, 3000, 3500, 4000, 4500, 5000, 5500, 6000, 6500, 7000, 7500, 8000, 8500, 9000, 9500, 10000, 15000, 20000, 25000, 30000, 35000, 40000, 45000, 50000, 55000, 60000, 65000, 70000, 75000, 80000, 85000, 90000, 95000, 100000, 110000, 120000, 130000, 140000, 150000, 160000, 170000, 180000, 190000, 200000, 210000, 220000, 230000, 240000, 250000, 260000, 270000, 280000, 290000, 300000, 310000, 320000, 330000, 340000, 350000, 360000, 370000, 380000, 390000, 400000, 410000, 420000, 430000, 440000, 450000, 460000, 470000, 480000, 490000, or 500000 NKG2D molecules.

In some embodiments of the cell populations provided herein, at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% of the gdT cells express NKp44. In some embodiments of the cell populations provided herein, the gdT cells express at least 400, 500, 600, 700, 800, 900, 1000, 1500, 2000, 2500, 3000, 3500, 4000, 4500, 5000, 5500, 6000, 6500, 7000, 7500, 8000, 8500, 9000, 9500, 10000, 15000, 20000, 25000, 30000, 35000, 40000, 45000, 50000, 55000, 60000, 65000, 70000, 75000, 80000, 85000, 90000, 95000, 100000, 110000, 120000, 130000, 140000, 150000, 160000, 170000, 180000, 190000, or 200000 NKp44 molecules per cell on average. In some embodiments, 1-100% of the gdT cells in the composition express 400-200000 NKp44 molecules per cell on average. In some embodiments of the cell populations provided herein, the NKp44-expressing gdT cells express at least 400, 500, 600, 700, 800, 900, 1000, 1500, 2000, 2500, 3000, 3500, 4000, 4500, 5000, 5500, 6000, 6500, 7000, 7500, 8000, 8500, 9000, 9500, 10000, 15000, 20000, 25000, 30000, 35000, 40000, 45000, 50000, 55000, 60000, 65000, 70000, 75000, 80000, 85000, 90000, 95000, 100000, 110000, 120000, 130000, 140000, 150000, 160000, 170000, 180000, 190000, or 200000 NKp44 molecules per cell on average. In some embodiments of the cell populations provided herein, the NKp44-expressing gdT cells each expresses at least 400, 500, 600, 700, 800, 900, 1000, 1500, 2000, 2500, 3000, 3500, 4000, 4500, 5000, 5500, 6000, 6500, 7000, 7500, 8000, 8500, 9000, 9500, 10000, 15000, 20000, 25000, 30000, 35000, 40000, 45000, 50000, 55000, 60000, 65000, 70000, 75000, 80000, 85000, 90000, 95000, 100000, 110000, 120000, 130000, 140000, 150000, 160000, 170000, 180000, 190000, or 200000 NKp44 molecules.

In some embodiments of the cell populations provided herein, at least 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% of the gdT cells express NKp46. In some embodiments of the cell populations provided herein, the gdT cells express at least 400, 500, 600, 700, 800, 900, 1000, 1500, 2000, 2500, 3000, 3500, 4000, 4500, 5000, 5500, 6000, 6500, 7000, 7500, 8000, 8500, 9000, 9500, 10000, 15000, 20000, 25000, 30000, 35000, 40000, 45000, 50000, 55000, 60000, 65000, 70000, 75000, 80000, 85000, 90000, 95000, 100000, 110000, 120000, 130000, 140000, 150000, 160000, 170000, 180000, 190000, or 200000 NKp46 molecules per cell on average. In some embodiments of the cell populations provided herein, the NKp46-expressing gdT cells express at least 400, 500, 600, 700, 800, 900, 1000, 1500, 2000, 2500, 3000, 3500, 4000, 4500, 5000, 5500, 6000, 6500, 7000, 7500, 8000, 8500, 9000, 9500, 10000, 15000, 20000, 25000, 30000, 35000, 40000, 45000, 50000, 55000, 60000, 65000, 70000, 75000, 80000, 85000, 90000, 95000, 100000, 110000, 120000, 130000, 140000, 150000, 160000, 170000, 180000, 190000, or 200000 NKp46 molecules per cell on average. In some embodiments, 4%-100% of the gdT cells in the composition express 400-200000 NKp46 molecules per cell on average. In some embodiments of the cell populations provided herein, the NKp46-expressing gdT cells each expresses at least 400, 500, 600, 700, 800, 900, 1000, 1500, 2000, 2500, 3000, 3500, 4000, 4500, 5000, 5500, 6000, 6500, 7000, 7500, 8000, 8500, 9000, 9500, 10000, 15000, 20000, 25000, 30000, 35000, 40000, 45000, 50000, 55000, 60000, 65000, 70000, 75000, 80000, 85000, 90000, 95000, 100000, 110000, 120000, 130000, 140000, 150000, 160000, 170000, 180000, 190000, or 200000 NKp46 molecules.

In some embodiments of the cell populations provided herein, at least 10%, 20%, 30%, 35%, 40%, 45%, 50%, 55%, 60%, 65%, 70%, 75%, or 80% of the gdT cells express CD107a. In some embodiments, at least 10% of the gdT cells express CD107a. In some embodiments, at least 20% of the gdT cells express CD107a. In some embodiments, at least 30% of the gdT cells express CD107a. In some embodiments, at least 40% of the gdT cells express CD107a. In some embodiments, at least 50% of the gdT cells express CD107a. In some embodiments, at least 60% of the gdT cells express CD107a. In some embodiments, at least 70% of the gdT cells express CD107a. In some embodiments, at least 80% of the gdT cells express CD107a. In some embodiments, about 10% to about 80% of the gdT cells express CD107a. In some embodiments, about 10% to about 70% of the gdT cells express CD107a. In some embodiments, about 10% to about 60% of the gdT cells express CD107a. In some embodiments, about 20% to about 80% of the gdT cells express CD107a. In some embodiments, about 20% to about 60% of the gdT cells express CD107a.

In some embodiments of the cell populations provided herein, the gdT cells express at least 400, 500, 600, 700, 800, 900, 1000, 1500, 2000, 2500, 3000, 3500, 4000, 4500, 5000, 5500, 6000, 6500, 7000, 7500, 8000, 8500, 9000, 9500, 10000, 15000, 20000, 25000, 30000, 35000, 40000, 45000, 50000, 55000, 60000, 65000, 70000, 75000, 80000, 85000, 90000, 95000, 100000, 110000, 120000, 130000, 140000, 150000, 160000, 170000, 180000, 190000, or 200000 CD107a molecules per cell on average. In some embodiments of the cell populations provided herein, at least 30%, 35%, 40%, 45%, 50%, 55%, 60%, 65%, 70%, 75%, or 80% of the gdT cells express CD107a. In some embodiments, the CD107a-expressing gdT cells express at least 400, 500, 600, 700, 800, 900, 1000, 1500, 2000, 2500, 3000, 3500, 4000, 4500, 5000, 5500, 6000, 6500, 7000, 7500, 8000, 8500, 9000, 9500, 10000, 15000, 20000, 25000, 30000, 35000, 40000, 45000, 50000, 55000, 60000, 65000, 70000, 75000, 80000, 85000, 90000, 95000, 100000, 110000, 120000, 130000, 140000, 150000, 160000, 170000, 180000, 190000, or 200000 CD107a molecules per cell on average. In some embodiments, 10-80% of the gdT cells in the composition express 400-200000 CD107a molecules per cell on average. In some embodiments, the CD107a-expressing gdT cells each expresses at least 400, 500, 600, 700, 800, 900, 1000, 1500, 2000, 2500, 3000, 3500, 4000, 4500, 5000, 5500, 6000, 6500, 7000, 7500, 8000, 8500, 9000, 9500, 10000, 15000, 20000, 25000, 30000, 35000, 40000, 45000, 50000, 55000, 60000, 65000, 70000, 75000, 80000, 85000, 90000, 95000, 100000, 110000, 120000, 130000, 140000, 150000, 160000, 170000, 180000, 190000, or 200000 CD107a molecules.

In some embodiments of the cell populations provided herein, at least 0.1% of the gdT cells express IFNγ. In some embodiments, at least 0.1%, 0.2%, 0.5%, 0.7%, 1%, 2%, 5%, 7%, 10%, 12%, 15%, 17%, 20%, 22%, 25%, 27%, 30%, 32%, 35%, 37%, 40%, 42%, 45%, 47%, 50%, 52%, 55%, 57%, 60%, 62%, 65%, 67%, 70%, 72%, 75%, 77%, 80%, 82%, 85%, 87%, 90%, 92%, 95%, 97%, or 100% of the gdT cells in the composition express IFNγ. In some embodiments of the cell populations provided herein, the gdT cells express at least 100, 200, 300, 400, 500, 600, 700, 800, 900, 1000, 1500, 2000, 2500, 3000, 3500, 4000, 4500, 5000, 5500, 6000, 6500, 7000, 7500, 8000, 8500, 9000, 9500, 10000, 15000, 20000, 25000, 30000, 35000, 40000, 45000, 50000, 55000, 60000, 65000, 70000, 75000, 80000, 85000, 90000, 95000, 100000, 110000, 120000, 130000, 140000, 150000, 160000, 170000, 180000, 190000, or 200000 IFNγ molecules per cell on average. In some embodiments of the cell populations provided herein, the IFNγ-expressing gdT cells express at least 100, 200, 300, 400, 500, 600, 700, 800, 900, 1000, 1500, 2000, 2500, 3000, 3500, 4000, 4500, 5000, 5500, 6000, 6500, 7000, 7500, 8000, 8500, 9000, 9500, 10000, 15000, 20000, 25000, 30000, 35000, 40000, 45000, 50000, 55000, 60000, 65000, 70000, 75000, 80000, 85000, 90000, 95000, 100000, 110000, 120000, 130000, 140000, 150000, 160000, 170000, 180000, 190000, or 200000 IFNγ molecules per cell on average. In some embodiments, 0.1%-100% of the gdT cells in the composition express 100-200000 IFNγ molecules per cell on average. In some embodiments of the cell populations provided herein, the IFNγ-expressing gdT cells each expresses at least 100, 200, 300, 400, 500, 600, 700, 800, 900, 1000, 1500, 2000, 2500, 3000, 3500, 4000, 4500, 5000, 5500, 6000, 6500, 7000, 7500, 8000, 8500, 9000, 9500, 10000, 15000, 20000, 25000, 30000, 35000, 40000, 45000, 50000, 55000, 60000, 65000, 70000, 75000, 80000, 85000, 90000, 95000, 100000, 110000, 120000, 130000, 140000, 150000, 160000, 170000, 180000, 190000, or 200000 IFNγ molecules.

In some embodiments of the cell populations provided herein, 10-100% of the gdT cells express Granzyme B. In some embodiments, at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% or 100% of the gdT cells in the composition express Granzyme B. In some embodiments, at least 25% of the cells express Granzyme B. In some embodiments of the cell populations provided herein, the gdT cells express at least 400, 500, 600, 700, 800, 900, 1000, 1500, 2000, 2500, 3000, 3500, 4000, 4500, 5000, 5500, 6000, 6500, 7000, 7500, 8000, 8500, 9000, 9500, 10000, 15000, 20000, 25000, 30000, 35000, 40000, 45000, 50000, 55000, 60000, 65000, 70000, 75000, 80000, 85000, 90000, 95000, 100000, 110000, 120000, 130000, 140000, 150000, 160000, 170000, 180000, 190000, or 200000 Granzyme B molecules per cell on average. In some embodiments of the cell populations provided herein, the Granzyme B-expressing gdT cells express at least 400, 500, 600, 700, 800, 900, 1000, 1500, 2000, 2500, 3000, 3500, 4000, 4500, 5000, 5500, 6000, 6500, 7000, 7500, 8000, 8500, 9000, 9500, 10000, 15000, 20000, 25000, 30000, 35000, 40000, 45000, 50000, 55000, 60000, 65000, 70000, 75000, 80000, 85000, 90000, 95000, 100000, 110000, 120000, 130000, 140000, 150000, 160000, 170000, 180000, 190000, or 200000 Granzyme B molecules per cell on average. In some embodiments, 30%-100% of the gdT cells in the composition express 400-200000 Granzyme B molecules per cell on average. In some embodiments of the cell populations provided herein, the Granzyme B-expressing gdT cells each expresses at least 400, 500, 600, 700, 800, 900, 1000, 1500, 2000, 2500, 3000, 3500, 4000, 4500, 5000, 5500, 6000, 6500, 7000, 7500, 8000, 8500, 9000, 9500, 10000, 15000, 20000, 25000, 30000, 35000, 40000, 45000, 50000, 55000, 60000, 65000, 70000, 75000, 80000, 85000, 90000, 95000, 100000, 110000, 120000, 130000, 140000, 150000, 160000, 170000, 180000, 190000, or 200000 Granzyme B molecules.

In some embodiments of the cell populations provided herein, 0-80% of the gdT cells express TIGIT. In some embodiments, at least 1%, 5%, 10%, 15%, 20%, 25%, 30%, 35%, 40%, 45%, 50%, 55%, or 60% of the gdT cells in the composition express TIGIT. In some embodiments of the cell populations provided herein, the gdT cells express at least 400, 500, 600, 700, 800, 900, 1000, 1500, 2000, 2500, 3000, 3500, 4000, 4500, 5000, 5500, 6000, 6500, 7000, 7500, 8000, 8500, 9000, 9500, 10000, 15000, 20000, 25000, 30000, 35000, 40000, 45000, 50000, 55000, 60000, 65000, 70000, 75000, 80000, 85000, 90000, 95000, 100000, 110000, 120000, 130000, 140000, 150000, 160000, 170000, 180000, 190000, or 200000 TIGIT molecules per cell on average. In some embodiments of the cell populations provided herein, the TIGIT-expressing gdT cells express at least 400, 500, 600, 700, 800, 900, 1000, 1500, 2000, 2500, 3000, 3500, 4000, 4500, 5000, 5500, 6000, 6500, 7000, 7500, 8000, 8500, 9000, 9500, 10000, 15000, 20000, 25000, 30000, 35000, 40000, 45000, 50000, 55000, 60000, 65000, 70000, 75000, 80000, 85000, 90000, 95000, 100000, 110000, 120000, 130000, 140000, 150000, 160000, 170000, 180000, 190000, or 200000 TIGIT molecules per cell on average. In some embodiments, 30%-100% of the gdT cells in the composition express 400-200000 TIGIT molecules per cell on average. In some embodiments of the cell populations provided herein, the TIGIT-expressing gdT cells each expresses at least 400, 500, 600, 700, 800, 900, 1000, 1500, 2000, 2500, 3000, 3500, 4000, 4500, 5000, 5500, 6000, 6500, 7000, 7500, 8000, 8500, 9000, 9500, 10000, 15000, 20000, 25000, 30000, 35000, 40000, 45000, 50000, 55000, 60000, 65000, 70000, 75000, 80000, 85000, 90000, 95000, 100000, 110000, 120000, 130000, 140000, 150000, 160000, 170000, 180000, 190000, or 200000 TIGIT molecules.

In some embodiments of the cell populations provided herein, 10-100% of the gdT cells express CD18. In some embodiments, at least 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% or 100% of the gdT cells in the composition express CD18. In some embodiments of the cell populations provided herein, the gdT cells express at least 400, 500, 600, 700, 800, 900, 1000, 1500, 2000, 2500, 3000, 3500, 4000, 4500, 5000, 5500, 6000, 6500, 7000, 7500, 8000, 8500, 9000, 9500, 10000, 15000, 20000, 25000, 30000, 35000, 40000, 45000, 50000, 55000, 60000, 65000, 70000, 75000, 80000, 85000, 90000, 95000, 100000, 110000, 120000, 130000, 140000, 150000, 160000, 170000, 180000, 190000, or 200000 CD18 molecules per cell on average. In some embodiments of the cell populations provided herein, the CD18-expressing gdT cells express at least 400, 500, 600, 700, 800, 900, 1000, 1500, 2000, 2500, 3000, 3500, 4000, 4500, 5000, 5500, 6000, 6500, 7000, 7500, 8000, 8500, 9000, 9500, 10000, 15000, 20000, 25000, 30000, 35000, 40000, 45000, 50000, 55000, 60000, 65000, 70000, 75000, 80000, 85000, 90000, 95000, 100000, 110000, 120000, 130000, 140000, 150000, 160000, 170000, 180000, 190000, or 200000 CD18 molecules per cell on average. In some embodiments, 30-100% of the gdT cells in the composition express 400-200000 CD18 molecules per cell on average. In some embodiments of the cell populations provided herein, the CD18-expressing gdT cells each expresses at least 400, 500, 600, 700, 800, 900, 1000, 1500, 2000, 2500, 3000, 3500, 4000, 4500, 5000, 5500, 6000, 6500, 7000, 7500, 8000, 8500, 9000, 9500, 10000, 15000, 20000, 25000, 30000, 35000, 40000, 45000, 50000, 55000, 60000, 65000, 70000, 75000, 80000, 85000, 90000, 95000, 100000, 110000, 120000, 130000, 140000, 150000, 160000, 170000, 180000, 190000, or 200000 CD18 molecules.

In some embodiments of the cell populations provided herein, 5-100% of the gdT cells express NKp30. In some embodiments, at least 5%, 6%, 7%, 8%, 9%, 10%, 15%, 20%, 25%, 30%, 35%, 40%, 45%, 50%, 55% 60%, 65%, 70%, 75%, 80%, 85%, 90%, 91%, 92%, 93%, 94%, 95%, 96%, 97%, 98%, 99% or 100% of the gdT cells in the composition express NKp30. In some embodiments of the cell populations provided herein, the gdT cells express at least 400, 500, 600, 700, 800, 900, 1000, 1500, 2000, 2500, 3000, 3500, 4000, 4500, 5000, 5500, 6000, 6500, 7000, 7500, 8000, 8500, 9000, 9500, 10000, 15000, 20000, 25000, 30000, 35000, 40000, 45000, 50000, 55000, 60000, 65000, 70000, 75000, 80000, 85000, 90000, 95000, 100000, 110000, 120000, 130000, 140000, 150000, 160000, 170000, 180000, 190000, or 200000 NKp30 molecules per cell on average. In some embodiments of the cell populations provided herein, the NKp30-expressing gdT cells express at least 400, 500, 600, 700, 800, 900, 1000, 1500, 2000, 2500, 3000, 3500, 4000, 4500, 5000, 5500, 6000, 6500, 7000, 7500, 8000, 8500, 9000, 9500, 10000, 15000, 20000, 25000, 30000, 35000, 40000, 45000, 50000, 55000, 60000, 65000, 70000, 75000, 80000, 85000, 90000, 95000, 100000, 110000, 120000, 130000, 140000, 150000, 160000, 170000, 180000, 190000, or 200000 NKp30 molecules per cell on average. In some embodiments, 30-100% of the gdT cells in the composition express 400-200000 NKp30 molecules per cell on average. In some embodiments of the cell populations provided herein, the NKp30-expressing gdT cells each expresses at least 400, 500, 600, 700, 800, 900, 1000, 1500, 2000, 2500, 3000, 3500, 4000, 4500, 5000, 5500, 6000, 6500, 7000, 7500, 8000, 8500, 9000, 9500, 10000, 15000, 20000, 25000, 30000, 35000, 40000, 45000, 50000, 55000, 60000, 65000, 70000, 75000, 80000, 85000, 90000, 95000, 100000, 110000, 120000, 130000, 140000, 150000, 160000, 170000, 180000, 190000, or 200000 NKp30 molecules.

In some embodiments of the cell populations provided herein, 1-20% of the gdT cells express CCR7. In some embodiments, at least 1%, 2%, 5%, 10%, 12%, 15%, or 20% of the gdT cells in the composition express CCR7. In some embodiments of the cell populations provided herein, the gdT cells express at least 400, 500, 600, 700, 800, 900, 1000, 1500, 2000, 2500, 3000, 3500, 4000, 4500, 5000, 5500, 6000, 6500, 7000, 7500, 8000, 8500, 9000, 9500, 10000, 15000, 20000, 25000, 30000, 35000, 40000, 45000, 50000, 55000, 60000, 65000, 70000, 75000, 80000, 85000, 90000, 95000, 100000, 110000, 120000, 130000, 140000, 150000, 160000, 170000, 180000, 190000, or 200000 CCR7 molecules per cell on average. In some embodiments of the cell populations provided herein, the CCR7-expressing gdT cells express at least 400, 500, 600, 700, 800, 900, 1000, 1500, 2000, 2500, 3000, 3500, 4000, 4500, 5000, 5500, 6000, 6500, 7000, 7500, 8000, 8500, 9000, 9500, 10000, 15000, 20000, 25000, 30000, 35000, 40000, 45000, 50000, 55000, 60000, 65000, 70000, 75000, 80000, 85000, 90000, 95000, 100000, 110000, 120000, 130000, 140000, 150000, 160000, 170000, 180000, 190000, or 200000 CCR7 molecules per cell on average. In some embodiments, 30-100% of the gdT cells in the composition express 400-200000 CCR7 molecules per cell on average. In some embodiments of the cell populations provided herein, the CCR7-expressing gdT cells each expresses at least 400, 500, 600, 700, 800, 900, 1000, 1500, 2000, 2500, 3000, 3500, 4000, 4500, 5000, 5500, 6000, 6500, 7000, 7500, 8000, 8500, 9000, 9500, 10000, 15000, 20000, 25000, 30000, 35000, 40000, 45000, 50000, 55000, 60000, 65000, 70000, 75000, 80000, 85000, 90000, 95000, 100000, 110000, 120000, 130000, 140000, 150000, 160000, 170000, 180000, 190000, or 200000 CCR7 molecules.

In some embodiments of the cell populations provided herein, 0.5%-100% of the gdT cells express CD25. In some embodiments, at least 0.5%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% or 100% of the gdT cells in the composition express CD25.

In some embodiments of the cell populations provided herein, 30-100% of the gdT cells express CD38. In some embodiments, at least 30%, 35%, 40%, 45%, 50%, 55%, 60%, 65%, 70%, 75%, 80%, 85%, 90%, 91%, 92%, 93%, 94%, 95%, 96%, 97%, 98%, 99% or 100% of the gdT cells in the composition express CD38.

In some embodiments of the cell populations provided herein, 0-10% of the gdT cells express CD36. In some embodiments, 0.1%-10% of the gdT cells in the composition express CD36. In some embodiments, at least 0.01%, 0.05%, 0.1%, 0.25%, 0.5%, 0.75%, 1%, 2.5%, 5%, 7.5%, or 10% of the gdT cells in the composition express CD36.

In some embodiments of the cell populations provided herein, 0-10% of the gdT cells express CD103. In some embodiments, at least 0.05%, 0.1%, 0.5%, 1%, 5%, or 10% of the gdT cells in the composition express CD103.

In some embodiments of the cell populations provided herein, 1-60% of the gdT cells express PD-1. In some embodiments, at least 1%, 2%, 3%, 4%, 5%, 6%, 7%, 8%, 9%, 10%, 12%, 15%, 7%, 20%, 22%, 25%, 27% 30%, 32%, 35%, 37%, 40%, 42%, 45%, 47%, 50%, 52%, 55%, 57% or 60% of the gdT cells in the composition express PD-1.

In some embodiments of the cell populations provided herein, 30-100% of the gdT cells can mediate an ADCC response. In some embodiments of the cell populations provided herein, at least 35%, 40%, 45%, 50%, 55%, 60%, 65%, 70%, 75%, 80%, 85%, 90%, 95%, or 100% of the gdT cells can mediate an ADCC response. In some embodiments of the cell populations provided herein, at least 5×10⁷, 6×10⁷, 7×10⁷, 7.5×10⁷, 8×10⁷, 9×10⁷, 1×10⁸, 2×10⁸, 2.5×10⁸, 3×10⁸, 4×10⁸, 5×10⁸, 6×10⁸, 7×10⁸, 7.5×10⁸, 8×10⁸, 9×10⁸, 1×10⁹, 2×10⁹, 2.5×10⁹, 3×10⁹, 4×10⁹, 5×10⁹, 6×10⁹, 7×10⁹, 7.5×10⁹, 8×10⁹, 9×10⁹, 1×10¹⁰, 2×10¹⁰, 2.5×10¹⁰, 3×10¹⁰, 3.5×10¹⁰, 4×10¹⁰, 4.5×10¹⁰, 5×10¹⁰, 5.5×10¹⁰, 6×10¹⁰, 6.5×10¹⁰, 7×10¹⁰, 7.5×10¹⁰, 8×10¹⁰, 8.5×10¹⁰, 9×10¹⁰, 9.5×10¹⁰, or 1×10¹¹ of the gdT cells can mediate an ADCC response.

In some embodiments, provided herein are populations of cells comprising at least 70% gdT cells, wherein (1) the gdT cells express at least 400 DNAM-1 molecules per cell on average; (2) at least 30% of the gdT cells are CD69⁺; or both (1) and (2). In some embodiments, the cell populations provided herein comprise at least 70% gdT cells, wherein (1) the gdT cells express at least 400 DNAM-1 molecules per cell on average and (2) at least 30% of the gdT cells are CD69+. In some embodiments, the gdT cells express at least 500, at least 1000, at least 2000, or at least 3000 DNAM-1 molecules per cell on average. In some embodiments, at least 40%, at least 50%, at least 55%, at least 60%, at least 65%, at least 70%, at least 75%, or at least 80% of the gdT cells are CD69+. In some embodiments, at least 30%, at least 40%, at least 50%, at least 60%, at least 70%, or at least 80% of the gdT cells are TDEM cells.

In some embodiments of the cell population provided herein, after co-culture with target cells, (1) 40-100% of the gdT cells express TNFα, (2) 60-100% of the gdT cells express CD107a, or both (1) and (2); wherein the target cells are cancer cells, tumor cells, HIV or other virus-infected cells, fungi-infected cells, or protozoan-infected cells; or wherein the target cells are Raji, Daudi, K562, or other liquid tumor; or wherein the target cells are A549, SK-OV-3, BT-474, or other solid tumor.

In some embodiments of the cell populations provided herein, at least 60% of the gdT cells are activated to express CD107a after co-cultured with target cells. In some embodiments of the cell populations provided herein, at least 60%, 65%, 70%, 75%, 80%, 85%, 90%, 91%, 92%, 93%, 94%, 95%, 96%, 97%, 98%, or 99% of the gdT cells are activated to express CD107a after co-cultured with target cells.

In some embodiments of the cell populations provided herein, at least 40% of the gdT cells are activated to express TNF-u after co-cultured with target cells. In some embodiments of the cell population provided herein, at least 40%, 45%, 50%, 55%, 60%, 65%, 70%, 75%, 80%, 85%, 90%, 91%, 92%, 93%, 94%, 95%, 96%, 97%, 98%, or 99% of the gdT cells are activated to express TNF-u after co-cultured with target cells.

In some embodiments of the cell population provided herein, after co-culture with cancer cells, (1) at least 40% of the CD69+gdT cells express TNFα; (2) at least 40% of the CD69+gdT cells express Granzyme B; or both (1) and (2). In some embodiments, after co-culture with cancer cells, at least 40% of the CD69+gdT cells express TNFα. In some embodiments, upon co-culture with cancer cells, at least 40%, 45%, 50%, 55%, 60%, 65%, 70%, 75%, 80%, 85%, 90%, 91%, 92%, 93%, 94%, 95%, 96%, 97%, 98%, 99% or 100% of the CD69+gdT cells express TNFα. In some embodiments, upon co-culture with cancer cells, at least 40% of the CD69+gdT cells express Granzyme B. In some embodiments, upon co-culture with cancer cells, at least 40%, 45%, 50%, 55%, 60%, 65%, 70%, 75%, 80%, 85%, 90%, 91%, 92%, 93%, 94%, 95%, 96%, 97%, 98%, 99% or 100% of the CD69⁺ gdT cells express Granzyme B.

As a person of ordinary skill in the art would understand, a wide variety of combinations and permutations of various aspects of the markers disclosed herein can be used to characterize the cell populations described herein. Such combinations and permutations are expressly contemplated as within the scope of this disclose. Some are exemplified below.

In some embodiments of the cell populations provided herein, the gdT cells express (1) at least 400 CD56 molecules per cell on average; (2) at least 400 CD16 molecules per cell on average; (3) at least 400 NKG2D molecules per cell on average; (4) at least 400 CD107a molecules per cell on average; (5) at most 2800 PD-1 molecules per cell on average; (6) at least 5000 DNAM-1 molecules per cell on average; or (7) at least 400 CD69 molecules per cell on average; or any combination thereof.

In some embodiments of the cell populations provided herein, the gdT cells express (1) about 30000 to about 70000 CD69 molecules per cell on average; (2) about 60000 to about 80000 CD56 molecules per cell on average; (3) the gdT cells express about 80000 to about 90000 NKG2D molecules per cell on average; (4) the gdT cells express about 100000 to about 300000 DNAM-1 molecules per cell on average; or any combination thereof.

In some embodiments, the cell populations provided herein comprise (1) 40-100% CD69+cells; (2) 50-80% CD56+cells; (3) 20-90% CD16+cells; (4) 90-100% NKG2D+cells; (5) 20-60% CD107a+ cells; or (6) 90-100% DNAM-1+cells; or any combination thereof.

In some embodiments of the cell populations provided herein, (1) at least 95% of the CD69+gdT cells express DNAM-1; (2) at least 25% of the CD69+gdT cells express Granzyme B; or both (1) and (2).

In some embodiments of the cell populations provided herein, at least 95%, 96%, 97%, 98%, or 99% of the cells express CD3. In some embodiments of the cell populations provided herein, at least 95%, 96%, 97%, 98%, or 99% of the cells express NKG2D. In some embodiments of the cell populations provided herein, at least 50%, 55%, 60%, 65%, 70%, 75%, 80%, 85%, 90%, or 95% of the cells express CD107a. In some embodiments of the cell populations provided herein, at most 25%, 20%, 15%, 10%, or 5% of the cells express PD-1. In some embodiments of the cell populations provided herein, (1) at least 95%, 96%, 97%, 98%, or 99% of the cells express CD3; (2) at least 95%, 96%, 97%, 98%, or 99% of the cells express NKG2D; (3) at least 50%, 55%, 60%, 65%, 70%, 75%, 80%, 85%, 90%, or 95% of the cells express CD107a; (4) at most 25%, 20%, 15%, 10%, or 5% of the cells express PD-1; or any combination of (1)-(4).

In some embodiments of the cell populations provided herein, (1) at least 40% of the cells express CD56; (2) at least 30% of the cells express CD16; (3) at least 50% of the cells express NKG2D; (4) at least 30% of the cells express CD107a; or (5) at most 25% of the cells express PD-1; or any combination thereof.

In some embodiments of the cell populations provided herein, (1) at least 4% of the gamma delta T cells express at least 400 NKp46 molecules per cell; (2) at least 10% of the gamma delta T cells express at least 400 CD56 molecules per cell; (3) at least 10% of the gamma delta T cells express at least 400 CD16 molecules per cell; (4) at least 30% of the gamma delta T cells express at least 400 NKG2D molecules per cell; (5) at least 1% of the gamma delta T cells express at least 400 NKp44 molecules per cell; (6) 0-100% of the gamma delta T cells express CD25; (7) 30-100% of the gamma delta T cells express CD38; (8) 0-60% of the gamma delta T cells express PD-1; (9) 5-100% of the gamma delta T cells express NKp30; (10) 10-100% of the gamma delta T cells express CD18; (11) 0-80% of the gamma delta T cells express TIGIT; (12) 30-100% of the gamma delta T cells express DNAM-1; (13) 0-10% of the gamma delta T cells express CD36; (14) 0-10% of the gamma delta T cells express CD103; (15) 1-20% of the gamma delta T cells express CCR7; (16) 0-100% of the gamma delta T cells express IFNγ; (17) 10-100% of the gamma delta T cells express Granzyme B; (18) 30-100% of the gamma delta T cells are CD3+Vδ2⁺; (19) 30-100% of the gamma delta T cells are capable of mediating an antibody-dependent cell-mediated cytotoxicity (ADCC) response; or (20) at least 80% of the gamma delta T cells express at least 400 CD69 molecules per cell; or any combination about (1)-(2).

In some embodiments of the cell populations provided herein, (1) at least 4% of the gamma delta T cells express NKp46, wherein the NKp46-expressing gamma delta T cells express at least 400 NKp46 molecules per cell on average; (2) at least 10% of the gamma delta T cells express CD56, wherein the CD56-expressing gamma delta T cells express at least 400 CD56 molecules per cell on average; (3) at least 10% of the gamma delta T cells express CD16, wherein the CD16-expressing gamma delta T cells express at least 400 CD16 molecules per cell on average; (4) at least 30% of the gamma delta T cells express NKG2D, wherein the NKG2D-expressing gamma delta T cells express at least 40 NKG2D molecules per cell on average; (5) at least 1% of the gamma delta T cells express NKp44, wherein the NKp44-expressing gamma delta T cells express at least 400 NKp44 molecules per cell on average; or (6) at least 80% of the gamma delta T cells express CD69, wherein the CD69-expressing gamma delta T cells express at least 400 CD69 molecules per cell on average; or any combination of (1)-(6).

In some embodiments, the cell populations provided herein are isolated. In some embodiments, the cell populations can be isolated from the human or animal body. In some embodiments, the isolated cell populations are substantially free of one or more cell populations that are associated with said cell population in vivo.

The cell populations disclosed herein can be obtained by the culturing methods described herein. See more details in section 5.1. In some embodiments, for example, the cell populations disclosed herein have been cultured ex vivo for 20 days or less since the source cell population from which the cell population is derived or obtained from a single donor. In some embodiments, the cell populations provided herein have not been positively selected for gdT cells. In some embodiments, the cell populations provided herein have not been positively selected for CD69+cells. In some embodiments, the cell populations provided herein have not been positively selected for any marker. In some embodiments, the cell population is free of feeder cells (e.g., transformed cells) or foreign antigen (e.g., microbial components).

### 5.2.1 Modified Cell Populations

The cell populations provided herein can be further modified to enhance their therapeutic potential. Accordingly, in some embodiments, methods provided herein further comprise adding a targeting moiety to the surface of the cells in the resulting cell population. Provided herein are also cell populations enriched in gdT cells wherein at least 10% of gdT cells comprise a targeting moiety complexed to the cell surface. In some embodiments, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% or 100% of the gdT cells in the cell populations provided herein comprise at least a targeting moiety that exhibits specific binding to a biological marker on a target cell.

The “targeting moiety” as used herein can distinguish target from non-target by exhibiting preferential interaction or binding toward the target. In some embodiments, the targeting moieties exhibit specific binding to a biological marker on a target cell. A targeting moiety can be selected based on having, or produced to have, a binding affinity for a desired target, such as a biological marker on a target cell (see U.S. Pat. No. 10,744,207). In some embodiments, the biological marker can be a tumor antigen or cancer antigen.

The term “specifically binds,” as used herein, means that a molecule interacts more frequently, more rapidly, with greater duration, with greater affinity, or with some combination of the above to the target molecule (e.g., epitope or protein) than with alternative substances. A targeting moiety (e.g., antibody) that specifically binds a target molecule (e.g., antigen) can be identified, for example, by immunoassays, ELISAs, Bio-Layer Interferometry (“BLI”), SPR (e.g., Biacore), or other techniques known to those of skill in the art. Typically, a specific reaction will be at least twice background signal or noise and can be more than 10 times background. See, e.g., Paul, ed., 1989, Fundamental Immunology Second Edition, Raven Press, New York at pages 332-336 for a discussion regarding antibody specificity. A targeting moiety that specifically binds a target molecule can bind the target molecule at a higher affinity than its affinity for a different molecule. In some embodiments, a targeting moiety that specifically binds a target molecule can bind the target molecule with an affinity that is at least 20 times greater, at least 30 times greater, at least 40 times greater, at least 50 times greater, at least 60 times greater, at least 70 times greater, at least 80 times greater, at least 90 times greater, or at least 100 times greater, than its affinity for a different molecule. In some embodiments, a targeting moiety that specifically binds a particular target molecule binds a different molecule at such a low affinity that binding cannot be detected using an assay described herein or otherwise known in the art. Because of homology within certain regions of polypeptide sequences of different proteins and structure similarities of different molecules, specific binding can include a molecule that recognizes more than one target. It is understood that, in some embodiments, a targeting moiety (e.g., antibody) that specifically binds a first target may or may not specifically bind a second target. As such, “specific binding” does not necessarily require (although it can include) exclusive binding, i.e., binding to a single target. Thus, a targeting moiety (e.g., antibody) can, in some embodiments, specifically bind more than one target.

The term “binding affinity” as used herein generally refers to the strength of the sum total of noncovalent interactions between a targeting moiety and a target molecule (e.g., antigen). The binding of a targeting moiety and a target molecule is a reversible process, and the affinity of the binding is typically reported as an equilibrium dissociation constant (K_{D}). K_{D} is the ratio of a dissociation rate (k_{off} or k_{d}) to the association rate (kₒₙ or kₐ). The lower the K_{D} of a binding pair, the higher the affinity. A variety of methods of measuring binding affinity are known in the art, any of which can be used for purposes of the present disclosure. In some embodiments, the “K_{D}” or “K_{D} value” can be measured by assays known in the art, for example by a binding assay. The K_{D} can be measured in a radiolabeled antigen binding assay (RIA) (Chen, et al., (1999) J. Mol Biol 293:865-881). The K_{D} or K_{D} value can also be measured by using biolayer interferometry (BLI) using, for example, the Gator system (Probe Life), or the Octet-96 system (Sartorius AG). The K_{D} or K_{D} value can also be measured by using surface plasmon resonance assays (SPR) by Biacore, using, for example, a BIAcore™-2000 or a BIAcore™-3000 BIAcore, Inc., Piscataway, NJ). In some embodiments, “specifically binds” means, for instance, that a targeting moiety binds a molecule target with a K_{D} of about 0.1 mM or less. In some embodiments, “specifically binds” means that a targeting moiety binds a target with a K_{D} of at about 10 μM or less or about 1 μM or less. In some embodiments, “specifically binds” means that a targeting moiety binds a target with a K_{D} of at about 0.1 μM or less, about 0.01 μM or less, or about 1 nM or less.

In some embodiments, the targeting moiety binds to the biological marker with a K_{D} of 10⁻⁶ M or less, 10⁻⁷ M or less, 10⁻⁸ M or less, 5×10⁻⁹ M or less, 10⁻⁹ M or less, 5×10⁻¹⁰ M or less, 10⁻¹⁰ M or less, 5×10⁻¹¹ M or less, 10⁻¹¹ M or less, 5×10⁻¹² M or less, or 10⁻¹² M or less; or ranging from 10⁻¹² M to 10⁻⁷ M, from 10⁻¹¹ M to 10⁻⁷ M, from 10⁻¹⁰ M to 10⁻⁷ M, from 10⁻⁹ M to 10⁻⁷ M, from 10⁻⁸ M to 10⁻⁷ M, from 10⁻¹⁰ M to 10⁻⁸ M, from 10⁻⁹ M to 10⁻⁸ M, from 10⁻¹¹ M to 10⁻⁹ M, or from 10⁻¹⁰ M to 10⁻⁹ M. In some embodiments, the K_{D} is less than 1, 5, 10, 11, 15, 20, 21, 25, 30, 31, 35, 40, 41, 45, 50, 51, 55, 60, 61, 65, 70, 71, 75, 80, 81, 85, 90, 91, 95, 100, 101, 105, 110, 111, 115, 120, 121, 125, 130, 131, 135, 140, 141, 145, 150, 151, 155, 160, 161, 165, 170, 171, 175, 180, 181, 185, 190, 191, 195, 200, 201, 205, 210, 211, 215, 220, 221, 225, 230, 231, 235, 240, 241, or 245 nM. In some embodiments, the K_{D} is less than 250 nM.

Biological markers to which a targeting moiety can be directed include cell surface markers. Non-limiting examples of cell surface markers include carbohydrates; glycolipids; glycoproteins; CD (cluster of differentiation) antigens present on cells of a hematopoietic lineage (e.g., CD2, CD4, CD8, CD21, etc.); γ-glutamyltranspeptidase; an adhesion protein (e.g., ICAM-1, ICAM-2, ELAM-1, VCAM-1); hormone, growth factor, cytokine, and other ligand receptors; ion channels; and the membrane-bound form of an immunoglobulin p chain. In some embodiments, the biological marker associated with a target cell is present on the surface of a target cells at about or less than about 100000, 50000, 10000, 5000, 1000, 750, 500, 100, 50, or fewer copies per cell. In some embodiments, the average density of a biological marker associated with the surface of a target cell in a population of target cells is about or less than about 100000, 50000, 10000, 5000, 1000, 750, 500, 100, 50, or fewer copies per cell. In some embodiments, the biological marker is associated with a target cell by way of increased concentration of the marker in a fluid surrounding the target cell or a tissue in which it resides than is found in fluid or tissue more distant from the target cell, such as where a cell secretes the biological marker. Of particular interest are biological markers associated with a disease or disease state; of particular further interest are disease-related biological markers expressed by a target cell (such as an abnormal cell) which is associated with the disease or the disease state.

A vast variety of disease-related biological markers have been identified, and the corresponding targeting moieties have been generated, such as targeting moieties direct to alfa-fetoprotein (AFP), C-reactive protein (CRP), cancer antigen-50 (CA-50), cancer antigen-125 (CA-125) associated with ovarian cancer, cancer antigen 15-3 (CA15-3) associated with breast cancer, cancer antigen-19 (CA-19) and cancer antigen-242 associated with gastrointestinal cancers, carcinoembryonic antigen (CEA), carcinoma associated antigen (CAA), chromogranin A, epithelial mucin antigen (MC5), human epithelium specific antigen (HEA), Lewis(a) antigen, melanoma antigen, melanoma associated antigens 100, 25, and 150, mucin-like carcinoma-associated antigen, multidrug resistance related protein (MRPm6), multidrug resistance related protein (MRP41), Neu oncogene protein (C-erbB-2), neuron specific enolase (NSE), P-glycoprotein (mdrl gene product), multidrug-resistance-related antigen, p170, multidrug-resistance-related antigen, prostate specific antigen (PSA), CD56, and NCAM (see U.S. Pat. No. 10,744,207).

In some embodiments, the biological marker is a glycolipid, glycoprotein, cluster of differentiation antigen present on cells of a hematopoietic lineage, gamma-glutamyltranspeptidase, adhesion protein, hormone, growth factor, cytokine, ligand receptor, ion channel, membrane-bound form of an immunoglobulin μ chain, alfa-fetoprotein, C-reactive protein, chromogranin A, epithelial mucin antigen, human epithelium specific antigen, Lewis(a) antigen, multidrug resistance related protein, Neu oncogene protein, neuron specific enolase, P-glycoprotein, multidrug-resistance-related antigen, p170, multidrug-resistance-related antigen, prostate specific antigen, NCAM, ganglioside molecule, MART-1, heat shock protein, sialyl-Tn, tyrosinase, MUC-1, HER-2/neu, KSA, PSMA, p53, RAS, EGF-R, VEGF, or MAGE.

In some embodiments, the targeting moiety is a peptide, protein, or aptamer. In some embodiments, the targeting moiety can comprise an antibody or antigen binding fragment that specifically binds to a biological marker on a target cell. The biological marker can be any biological marker disclosed herein or otherwise known in the art. In some embodiments, the targeting moiety can comprise an antibody or antigen binding fragment that specifically binds to a tumor antigen or cancer antigen. Methods provided herein further comprise adding an antibody or antigen-binding unit thereof that specifically binds a tumor antigen to the surface of the cells.

In some embodiments, the targeting moiety comprises an antibody or antigen-binding unit that specifically binds to a biological marker on the target cell. As understood in the art, the term “antibody,” and its grammatical equivalents as used herein refer to an immunoglobulin molecule that recognizes and specifically binds a target, such as a protein, polypeptide, peptide, carbohydrate, polynucleotide, lipid, or a combination of any of the foregoing, through at least one antigen-binding site wherein the antigen-binding site is usually within the variable region of the immunoglobulin molecule. As used herein, the term encompasses intact polyclonal antibodies, intact monoclonal antibodies, single-domain antibodies (sdAbs; e.g., camelid antibodies, alpaca antibodies), single-chain Fv (scFv) antibodies, heavy chain antibodies (HCAbs), light chain antibodies (LCAbs), multispecific antibodies, bispecific antibodies, monospecific antibodies, monovalent antibodies, and any other modified immunoglobulin molecule comprising an antigen-binding site (e.g., dual variable domain immunoglobulin molecules) as long as the antibodies exhibit the desired biological activity. Antibodies also include, but are not limited to, mouse antibodies, camel antibodies, chimeric antibodies, humanized antibodies, and human antibodies. An antibody can be any of the five major classes of immunoglobulins: IgA, IgD, IgE, IgG, and IgM, or subclasses (isotypes) thereof (e.g., IgG1, IgG2, IgG3, IgG4, IgA1 and IgA2), based on the identity of their heavy-chain constant domains referred to as alpha, delta, epsilon, gamma, and mu, respectively. Unless expressly indicated otherwise, the term “antibody” as used herein include “antigen-binding unit” of intact antibodies. The term “antigen-binding unit” as used herein refers to a portion or fragment of an intact antibody that is the antigenic determining variable region of an intact antibody. Examples of antigen-binding unit include, but are not limited to, Fab, Fab′, F(ab′)2, Fv, linear antibodies, single chain antibody molecules (e.g., scFv), heavy chain antibodies (HCAbs), light chain antibodies (LCAbs), disulfide-linked scFv (dsscFv), diabodies, tribodies, tetrabodies, minibodies, dual variable domain antibodies (DVD), single variable domain antibodies (sdAbs; e.g., camelid antibodies, alpaca antibodies), and single variable domain of heavy chain antibodies (VHH), and bispecific or multispecific antibodies formed from antibody fragments. In some embodiment, the targeting moiety comprising an antigen-binding unit is a monoclonal antibody of an IgG subtype.

In some embodiments, the targeting moiety is an antibody or antigen-binding unit that specifically binds s cancer antigen. The cancer antigen can be selected from the group consisting of HER2/neu (ERBB2), HER3 (ERBB3), EGFR, VEGF, VEGFR2, GD2, CTLA4, CD19, CD20, CD22, CD30, CD33 (Siglec-3), CD52 (CAMPATH-1 antigen), CD326 (EpCAM), CA-125 (MUC16), MMP9, DLL3, CD274 (PD-L1), CEA, MSLN (mesothelin), CA19-9, CD73, CD205 (DEC205), CD51, c-MET, TRAIL-R2, IGF-1R, CD3, MIF, folate receptor alpha (FOLR1), CSF1, OX-40, CD137, TfR, MUC1, CD25 (IL-2R), CD115 (CSF1R), IL1B, CD105 (Endoglin), KIR, CD47, CEA, IL-17A, DLL4, CD51, angiopoietin 2, neuropilin-1, CD37, CD223 (LAG-3), CD40, LIV-1 (SLC39A6), CD27 (TNFRSF7), CD276 (B7-H3), Trop2, Claudin1 (CLDN1), PSMA, TIM-1 (HAVcr-1), CEACAM5, CD70, LY6E, BCMA, CD135 (FLT3), APRIL, TF(F3), nectin-4, FAP, GPC3, FGFR3, a killer-cell immunoglobulin-like receptors (KIRs), a TNF receptor protein, an immunoglobulin protein, a cytokine receptor, an integrin, and activating NK cell receptors.

In some embodiments, the targeting moiety comprises an anti-CD20 antibody (e.g., rituximab). In some embodiments, the targeting moiety comprises an anti-HER2 antibody (e.g., trastuzumab).

### 5.2.2 ACE Cells

In some embodiments, the targeting moiety is not produced by the gdT cells. In some embodiments, the targeting moiety is complexed to the cell surface via the interaction between a first linker conjugated to the targeting moiety and a second linker conjugated to the cell surface. The gdT cells having a targeting moiety complexed to the cell surface via the interaction between a first linker conjugated to the targeting moiety and a second linker conjugated to the cell surface are referred to as “ACE-gdT cells.”

In some embodiments, the targeting moiety and the surface of the cell is separated by a length of 1 nm to 400 nm. In some embodiments, the targeting moiety and the surface of the cell is separated by a distance of 2, 3, 4, 5, 6, 7, 8, 9, 10, 11, 12, 13, 14, 15, 16, 17, 18, 19, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 110, 120, 130, 140, 150, 160, 170, 180, 190, 200, 210, 220, 230,240, 250, 260, 270, 280, 290, 300, 310, 320, 330,340, 350, 360, 370, 380, or 390 nm. In some embodiments, the targeting moiety and the surface of the cell is separated by a length of 1 nm to 20 nm or 1 nm to 33 nm. In some embodiments, the targeting moiety and the surface of the cell is separated by a length of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, or 32 nm.

In some embodiments, the targeting moieties can be added to the gdT cells of cell populations provided herein via interaction between linkers that separately conjugated to the targeting moieties and the cells. In some embodiments, the first and second linkers are the same. In some embodiments, the first linker and the second linker are different. In some embodiments, the linker is an exogenous linker that is not produced by the cell to which it is conjugated.

In some embodiments, the first and second linkers comprise reactive groups that react with one another to form a covalent bond, and the targeting moiety is complexed to the cell surface via the covalent bond formed between the two reactive groups. Each reactive group can first be reacted directly with the entity to which it is attached (e.g., a targeting moiety or a therapeutic agent) to form a covalent bond (see U.S. Pat. No. 10,744,207). In some embodiments, the targeting moiety is conjugated to the first linker and/or the second linker via a coupling group. In some embodiments, the coupling group is an NHS ester or other activated ester, an alkyl or acyl halide, a bifunctional crosslinker, or maleimide group.

In some embodiments, the linkers can be a binding pair that interact non-covalently. Members of binding pairs specifically bind each other, including, but not limited to, a DNA binding domain and a target DNA; a leucine zipper and a target DNA; biotin and avidin; biotin and streptavidin; calmodulin binding protein and calmodulin; a hormone and a hormone receptor; lectin and a carbohydrate; a cell membrane receptor and a receptor ligand; an enzyme and a substrate; an antigen and an antibody; an agonist and an antagonist; polynucleotide (RNA or DNA) hybridizing sequences; an aptamer and a target; and a zinc finger and a target DNA.

In some embodiments, the two linkers bind to each other with a K_{D} of 10⁻⁶ M or less, 10⁻⁷ M or less, 10⁻⁸ M or less, 5×10⁻⁹ M or less, 10⁻⁹ M or less, 5×10⁻¹⁰ M or less, 10⁻¹⁰ M or less, 5×10⁻¹¹ M or less, 10⁻¹¹ M or less, 5×10⁻¹² M or less, or 10⁻¹² M or less; or ranging from 10⁻¹² M to 10⁻⁷ M, from 10⁻¹¹ M to 10⁻⁷ M, from 10⁻¹⁰ M to 10⁻⁷ M, from 10⁻⁹ M to 10⁻⁷ M, from 10⁻⁸ M to 10⁻⁷ M, from 10⁻¹⁰ M to 10⁻⁸ M, from 10⁻⁹ M to 10⁻⁸ M, from 10⁻¹¹ M to 10⁻⁹ M, or from 10⁻¹⁰ M to 10⁻⁹ M. In some embodiments, the K_{D} between the first linker and the second linker is less than 1, 5, 10, 11, 15, 20, 21, 25, 30, 31, 35, 40, 41, 45, 50, 51, 55, 60, 61, 65, 70, 71, 75, 80, 81, 85, 90, 91, 95, 100, 101, 105, 110, 111, 115, 120, 121, 125, 130, 131, 135, 140, 141, 145, 150, 151, 155, 160, 161, 165, 170, 171, 175, 180, 181, 185, 190, 191, 195, 200, 201, 205, 210, 211, 215, 220, 221, 225, 230, 231, 235, 240, 241, or 245 nM. In some embodiments, the two linkers have a binding affinity (K_{D}) less than 250 nM.

The interaction between the first linker and the second linker can be direct or indirect. In some embodiments, the first and second linker interact directly. In general, a direct interaction is an interaction that does not require interaction with an intermediate compound. In some embodiments, the first and second linker interact indirectly. In general, an indirect interaction is mediated by one or more intermediate compounds. An intermediate compound can be of the same or different type as one or both linkers. In some embodiments, the first and second linkers interact indirectly via simultaneous interaction with an intermediate compound. For example, the first and second linkers can be the same antibody, which interact indirectly with one another by way of simultaneously binding the same antigen (one or more copies) as the intermediate compound.

In some embodiments, the first linker is a first polynucleotide, and the second linker is a second polynucleotide. In some embodiments, the first polynucleotide is a compound comprised of deoxyribonucleotides, ribonucleotides, or analogs thereof, or any combination thereof. In some embodiments, the second polynucleotide is a compound comprised of deoxyribonucleotides, ribonucleotides, or analogs thereof, or any combination thereof (see U.S. Pat. No. 10,744,207). In some embodiments, at least one of the two polynucleotides can be independently a DNA, an RNA or a peptide nucleic acid (PNA) molecule, or a combination thereof (see U.S. Pat. No. 10,744,207). In some embodiments, the first and second polynucleotides can be single strand DNAs (ssDNAs).

In some embodiments, (1) the first polynucleotide has 4 to 500 nucleotides, (2) the second polynucleotide has 4 to 500 nucleotides, or both (1) and (2). In some embodiments, the length of at the first and/or the second polynucleotide is 5, 10, 15, 20, 25, 30, 35, 40, 45, 50, 55, 60, 65, 70, 75, 80, 85, 90, 95, 100, 120, 140, 160, 180, 200, 300, 400 or 500 nt. In some embodiments, the first and/or the second polynucleotide has between 20-200 nucleotides. In some embodiments, the first and/or the second polynucleotide has between 20-100 nucleotides. In some embodiments, the first and/or the second polynucleotide has between 20-80 nucleotides. In some embodiments, the first and/or the second polynucleotide has between 20-60 nucleotides. In some embodiments, the first and/or the second polynucleotide has about 20 nucleotides. In some embodiments, the first and/or the second polynucleotide has about 40 nucleotides. In some embodiments, the first and/or the second polynucleotide has about 60 nucleotides.

The two polynucleotide linkers can interact directly or indirectly. In some embodiments, the first and second polynucleotides can interact directly, such as by hybridizing to one another via complementarity. In some embodiments, the first polynucleotide comprises a first single-stranded region, and the second polynucleotide comprises a second single-stranded region complementary to the first single-stranded region, wherein the targeting moiety is complexed to the surface of the cell via the interaction between the first single-stranded region and the second single-stranded region complementary to the first single-stranded region. In some embodiments, the first single-stranded region and the second single-stranded region are substantially or fully complementary to each other. In some embodiments, the first polynucleotide and the second polynucleotide are substantially or fully complementary to each other. For example, the two polynucleotides share at least about 10%, 20%, 30%, 40%, 50%, 60%, 70%, 80%, 90%, 95%, 96%, 97%, 98%, 99% or 100% complementarity. In some embodiments, linkers are designed to have about or less about 90%, 80%, 70%, 60%, 50%, 40%, 30%, 20%, 10%, or lower GC content. In some embodiments, the linkers are selected to have about or more than about, 10%, 20%, 30%, 40%, 50%, 60%, 70%, 80%, 90%, or more GC content. In some embodiments, linkers are designed to comprise or consist of sequences of 1, 2, 3, 4, 5, 6, 7, 8, 9, or 10 nucleotides repeated 1, 2, 3, 4, 5, 6, 7, 8, 9, 10, 15, or 20 times, or repeated until reaching the end of the linker (e.g. AAA . . . , or ATAT . . . ). In some embodiments, linkers are selected to have a Tm of about or more than about 30° C., 35° C., 40° C., 45° C., 50° C., 55° C., 60° C., 65° C., 70° C., 75° C., 80° C., 85° C., or higher (see U.S. Pat. No. 10,744,207).

In some embodiments, the first or/and second polynucleotide comprise a sequence selected from the table below, group consisting of 20-mer poly-CA, 20-mer poly-GGTT, SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO:20, SEQ ID NO:21, SEQ ID NO:22, SEQ ID NO:23, SEQ ID NO:24, SEQ ID NO:25, and SEQ ID NO: 26.

| SEQ ID | |
|---|---|
| NO: | Sequence |
| 1 | CACACACACACACACACACA |
| 2 | TCATACGACTCACTCTAGGG |
| 3 | AGTTACCATGACGTCAATTTCAG |
| 4 | TGTGTGTGTGTGTGTGTGTG |
| 5 | CCCTAGAGTGAGTCGTATGA |
| 6 | CTGAAATTGACGTCATGGTAACT |
| 7 | AAAAAAAAAAAAAAAAAAAA |
| 8 | TTTTTTTTTTTTTTTTTTTT |
| 9 | ACTGACTGACTGACTGACTG |
| 10 | CAGTCAGTCAGTCAGTCAGT |
| 11 | GTAACGATCCAGCTGTCACT |
| 12 | AGTGACAGCTGGATCGTTAC |
| 13 | ACTGATGGTAATCTGCACCT |
| 14 | AGGTGCAGATTACCATCAGT |
| 15 | AAAAAAAAAAAAAAAAAAAAAAAAAAAAAAAAAAAAAAAA |
| 16 | TTTTTTTTTTTTTTTTTTTTTTTTTTTTTTTTTTTTTTTT |
| 17 | ACTGACTGACTGACTGACTGACTGACTGACTGACTGACTG |
| 18 | CAGTCAGTCAGTCAGTCAGTCAGTCAGTCAGTCAGTCAGT |
| 19 | TGTGTGTGTGTGTGTGTGTGTGTGTGTGTGTGTGTGTGTG |
| 20 | CACACACACACACACACACACACACACACACACACACACA |
| 21 | GTAACGATCCAGCTGTCACTGTAACGATCCAGCTGTCACT |
| 22 | AGTGACAGCTGGATCGTTACAGTGACAGCTGGATCGTTAC |
| 23 | TCATACGACTCACTCTAGGGTCATACGACTCACTCTAGGG |
| 24 | CCCTAGAGTGAGTCGTATGACCCTAGAGTGAGTCGTATGA |
| 25 | ACTGATGGTAATCTGCACCTACTGATGGTAATCTGCACCT |
| 26 | AGGTGCAGATTACCATCAGTAGGTGCAGATTACCATCAGT |

In some embodiments, the first and second linkers are two polynucleotides that interact indirectly via interaction with an intermediate compound. In some embodiments, the intermediate compound is an adapter polynucleotide. An adapter polynucleotide can comprise DNA, RNA, nucleotide analogues, non-canonical nucleotides, labeled nucleotides, modified nucleotides, or combinations thereof. Adapter polynucleotides can be single-stranded, double-stranded, or partial duplex. In general, a partial-duplex adapter comprises one or more single-stranded regions and one or more double-stranded regions. Double-stranded adapters can comprise two separate oligonucleotides hybridized to one another (also referred to as an “oligonucleotide duplex”), and hybridization may leave one or more 3′ overhangs, one or more 5′ overhangs, one or more bulges resulting from mismatched and/or unpaired nucleotides, or any combination of these. An adapter polynucleotide that interacts with both the first linker polynucleotide and the second linker polynucleotide can comprise a contiguous backbone. For example, a first linker polynucleotide and a second linker polynucleotide can interact via complementarity with a different portion of an adapter polynucleotide. Alternatively, the first linker polynucleotide can hybridize to a first strand of a double-stranded linker, the second linker polynucleotide can hybridize to a second strand of a double-stranded linker, and the first and second strands of the adapter can hybridize with one another, such that the first and second linkers interact indirectly via sequence complementarity with the double-stranded adapter polynucleotide. An adapter polynucleotide can alternatively comprise a discontiguous backbone, such as when two or more double-stranded adapter polynucleotides (e.g., 2, 3, 4, 5, or more) hybridize in a chain, with the first linker polynucleotide hybridizing to one end of the chain and the second linker polynucleotide hybridizing to the other end of the chain (see U.S. Pat. No. 10,744,207).

A linker can be conjugated to a targeting moiety (e.g., an antibody) or therapeutic unit (e.g., a cell) by any suitable means known in the art. The linker can be conjugated via a covalent or a non-covalent linkage. In some embodiments, the linker is conjugated to a native functional group of a moiety (e.g., an antibody) or therapeutic unit, such as natively on a surface of a cell or a native group in a protein. The cell surface can include any suitable native functional group, such as amino acids and sugars. For example, reagents including maleimide, disulfide and the process of acylation can be used to form a direct covalent bond with a cysteine on a cell surface protein. Amide coupling can be used at an aspartamate and glutamate to form an amide bond. Diazonium coupling, acylation, and alkylation can be used at a tyrosine on the cell surface to form an amide bond linkage. Any of the amino acids (20 amino acids or any unnatural amino acids) can be used to form the direct covalent bond that is the attachment of the oligonucleotide with the cell surface. The 20 amino acids are isoleucine, leucine, lysine, methionine, phenylalanine, threonine, tryptophan, and valine (essential amino acids), and alanine, asparagine, aspartate, cysteine, glutamate, glutamine, glycine, proline, serine, and tyrosine, the nonessential amino acids, and also arginine and histidine. In some embodiments, the native functional group can be an amino acid such as lysine, cysteine, tyrosine, threonine, serine, aspartic acid, glutamic acid or tryptophan. In other embodiments, the native functional group is lysine. In some other embodiments, the native functional group can be an N-terminal serine or threonine (see U.S. Pat. No. 10,744,207).

In some embodiments, the linker can be conjugated to the targeting moiety or therapeutic unit using a coupling group. For example, the coupling group can be an activated ester (e.g., NHS ester, 1-ethyl-3-(3-dimethylaminopropyl) carbodiimide (EDC) ester, dicyclohexylcarbodiimide (DCC) ester, etc.), or an alkyl or acyl halide (e.g., —Cl, —Br, —I). In some embodiments, the activated ester is isolated and/or purified. In some embodiments, the activated ester is generated and/or used in situ. In some cases, the coupling group can directly conjugate to the therapeutic agent (e.g., surface of a cell used as a therapeutic agent) without pre-modification of the native functional group (e.g., amino acids). For example, the linker can be conjugated to the targeting moiety or therapeutic unit by formation of a bond (e.g., an amide or ester bond) with an amino acid on a targeting moiety (e.g., antibody, aptamer) or a cell surface. In some embodiments, the coupling group is an NHS ester, which reacts with a nucleophilic native functional group on the targeting moiety or therapeutic unit, resulting in an acylated product. For example, the native functional group can be an amine, which is conjugated via the NHS ester to form an amide. Alternatively, the native functional group can be a hydroxyl or a sulfhydryl group, which can be conjugated via the NHS ester to form an ester or a sulfhydryl ester linkage, respectively (see U.S. Pat. No. 10,744,207).

In some embodiments, the linker can be conjugated to the targeting moiety or therapeutic unit using a bifunctional crosslinker. The bifunctional crosslinker can comprise two different reactive groups capable of coupling to two different functional targets such as peptides, proteins, macromolecules, semiconductor nanocrystals, or substrate. The two reactive groups can be the same or different and include but are not limited to such reactive groups as thiol, carboxylate, carbonyl, amine, hydroxyl, aldehyde, ketone, active hydrogen, ester, sulfhydryl or photoreactive moieties. In some embodiments, a cross-linker can have one amine-reactive group and a thiol-reactive group on the functional ends. In other embodiments, the bifunctional crosslinker can be an NHS-PEO-Maleimide, which comprise an N-hydroxysuccinimide (NHS) ester and a maleimide group that allow covalent conjugation of amine- and sulfhydryl-containing molecules. Further examples of heterobifunctional cross-linkers that may be used to conjugate the linker to the targeting moiety or therapeutic unit include but are not limited to: amine-reactive+sulfhydryl-reactive crosslinkers, carbonyl-reactive+sulfhydryl-reactive crosslinkers, amine-reactive+photoreactive crosslinkers, sulfhydryl-reactive+photoreactive crosslinkers, carbonyl-reactive+photoreactive crosslinkers, carboxylate-reactive+photoreactive crosslinkers, and arginine-reactive+photoreactive crosslinkers (see U.S. Pat. No. 10,744,207).

Typical crosslinkers can be classified in the following categories (with exemplary functional groups): 1. amine-reactive: the cross-linker couples to an amine (NH2) containing molecule, e.g., isothiocyanates, isocyanates, acyl azides, NHS esters, sulfonyl chlorides, aldehydes and glyoxals, epoxides and oxiranes, carbonates, arylating agents, imidoesters, carbodiimides, anhydrides, alkynes; 2. thiol-reactive: the cross-linker couple to a sulfhydryl (SH) containing molecule, e.g., haloacetyl and alkyl halide derivates, maleimides, aziridines, acryloyl derivatives, arylating agents, thiol-disulfides exchange reagents; 3. carboxylate-reactive: the cross-linker couple to a carboxylic acid (COOH) containing molecule, e.g., diazoalkanes and diazoacetyl compounds, such as carbonyldiimidazoles and carbodiimides; 4. hydroxyl-reactive: the cross-linker couple to a hydroxyl (—OH) containing molecule, e.g., epoxides and oxiranes, carbonyldiimidazole, oxidation with periodate, N,N′-disuccinimidyl carbonate or N-hydroxylsuccimidyl chloroformate, enzymatic oxidation, alkyl halogens, isocyanates; 5. Aldehyde- and ketone-reactive: the cross-linker couple to an aldehyde (—CHO) or ketone (R2CO) containing molecule, e.g., hydrazine derivatives for schiff base formation or reduction amination; 6. Active hydrogen-reactive, e.g., diazonium derivatives for mannich condensation and iodination reactions; and 7. Photo-reactive, e.g., aryl azides and halogenated aryl azides, benzophenones, diazo compounds, diazirine derivatives (see U.S. Pat. No. 10,744,207).

Some reactive groups can react with several functional groups. Thus, each category has subcategories, each of these subcategories include a variety of chemicals. The applicable chemicals under each category are known in the art, for example, in BIOCONJUGATE TECHNIQUES by Greg T Hermanson, Academic Press, San Diego, 1996, which is hereby incorporated by reference.

Exemplary crosslinkers also include polyethylene glycol (PEG), also referred to as polyethyleneoxide (PEO). Spacers can be used as alternatives to reagents with purely hydrocarbon spacer arms. PEG spacers improve water solubility of reagent and conjugate, reduce the potential for aggregation of the conjugate, and increases flexibility of the crosslink, resulting in reduced immunogenic response to the spacer itself. By contrast to typical PEG reagents that contain heterogeneous mixtures of different PEG chain lengths, these PEO reagents are homogeneous compounds of defined molecular weight and spacer 5 arm length, providing greater precision in optimization and characterization of crosslinking applications. For example, succinimidyl-[(N-maleimidopropionamido)-hexaethyleneglycol] ester was used in the examples to make a stock solution by dissolving 5 mg of NHS-PEO6-maleimide (Pierce Biotechnology, Inc. Rockford, Ill. 61105).

In some embodiments, the conjugation can result in a carboxyl or a carbonyl group, or amino or thio equivalents thereof. Examples of such groups include but are not limited to ketones, imides, thiones, amides, imidamides, thioamides, esters, imidoesters, thioesters, carbamates, ureas, thioureas, carbonates, carbonimidates and carbonthioates. In some embodiments, the conjugation can result in a hydrazone or an oxime bond. In some embodiments, the conjugation may result in a disulfide bond. In some embodiments, the linker can be conjugated using Native Chemical Ligation (NCL) methods. Additional examples of linkers and coupling groups are disclosed in WO2010118235A1, which is hereby incorporated by reference.

In some embodiments, the linker comprises a PEG region or an NHS ester. In some embodiments, the targeting moiety is conjugated to the first linker (e.g., a polypeptide) via an NHS ester, an activated ester, an alkyl or acyl halide, a bifunctional crosslinker, or maleimide group.

An exemplary procedure of adding a targeting moiety to the surface of the cells in the resulting cell population is provided below. A person of ordinary skill would understand that variants of these procedures and other alternatives can be adopted to modify the cell populations described herein by adding targeting moieties to the cell surfaces.

In some embodiments, ACE-gdT cells are prepared using complementary polynucleotides as the linkers. An exemplary method can include: (A) prepare gdT-ssDNA conjugates by coupling a first ssDNA linker to gdT cells; (B) prepare targeting moiety-ssDNA conjugates by coupling a second ssDNA linker to the targeting moiety; and (C) prepare ACE-gdT cells by mixing the gdT-ssDNA conjugates and targeting moiety-ssDNA conjugates and allowing the complementary ssDNA linkers to hybridize.

For illustrative purposes, step (A) can include steps (a1)˜(a4): (a1) obtain a first ssDNA (e.g., SEQ ID NO:1, SEQ ID NO:2, or SEQ ID NO:3); (a2) modify the 5′ end of the first ssDNA with a thiol group (5′ end thiol-modified first ssDNA) to obtain the cell linker stock (see e.g., Zimmermann, J, 2010, Nat. Protoc. 5(6):975-985; also commercially available from Integrated DNA Technologies); (a3) mix 10-500 μL cell linker stock and 0.1-10 μL NHS-Maleimide (SMCC, Fisher Scientific) and incubate for 1-60 minute(s); and (a4) incubate the mixture obtained from Step (a3) with 1×10⁶-1×10⁹ gdT cells for 1-60 minutes.

Similarly, step (B) can include steps (b1)˜(b4): (b1) obtain a second ssDNA (e.g., SEQ ID NO:4, SEQ ID NO:5, or SEQ ID NO:6); (b2) modify the 5′ end of the second ssDNA with a thiol group (5′ end thiol-modified second ssDNA) to obtain the targeting moiety linker stock (see e.g., Zimmermann, J, 2010; also commercially available from Integrated DNA Technologies); (b3) mix 10-500 μL targeting moiety linker stock and 0.1-10 μL NHS-Maleimide (SMCC, Fisher Scientific) and incubate for 1-60 minute(s); and (b4) incubate the mixture obtained from Step (b3) with 10-100 μL targeting moiety stock (e.g., rituximab or trastuzumab) for 10 minutes to 3 hours.

In some embodiments, Step (C) can include mixing the gdT-ssDNA conjugates and 100-500 μL of targeting moiety-ssDNA conjugates to allow the complementary ssDNA linkers to form a complex.

### 5.2.3 Cells Expressing CARs and TCRs

In some embodiments, the targeting moiety is exogenously expressed on the surface of gdT cells provided herein as the extracellular domain of a receptor protein. The receptor protein can comprise an extracellular domain that comprises the targeting moiety, an intracellular domain and a transmembrane sequence. In some embodiments, the receptor protein is a chimeric antigen receptor (“CAR”). In some embodiments, the receptor protein is a T cell receptor (“TCR”).

### 5.2.3.1 Cars

In some embodiments, the receptor is a CAR, and gdT cells in the cell populations provided herein are modified to express a CAR. CARs are synthetic receptors that retarget immune cells (e.g., T cells) to tumor surface antigens (Sadelain et al., Nat. Rev. Cancer. 3(1):35-45 (2003); Sadelain et al., Cancer Discovery 3(4):388-398 (2013)). CARs are engineered receptors that provide both antigen binding and immune cell activation functions. CARs can be used to graft the specificity of an antibody, such as a monoclonal antibody, onto an immune cell such as gdT cells. First-generation receptors link an antibody-derived tumor-binding element, such as an scFv, that is responsible for antigen recognition to either CD3zeta or Fc receptor signaling domains, which trigger T-cell activation. The advent of second-generation CARs, which combine activating and costimulatory signaling domains, has led to encouraging results in patients with chemorefractory B-cell malignancies (Brentjens et al., Science Translational Medicine 5(177):177ra38 (2013); Brentjens et al., Blood 118(18):4817-4828 (2011); Davila et al., Science Translational Medicine 6(224):224ra25 (2014); Grupp et al., N. Engl. J. Med. 368(16):1509-1518 (2013); Kalos et al., Science Translational Medicine 3(95):95ra73 (2011)). The extracellular antigen-binding domain of a CAR is usually derived from a monoclonal antibody (mAb) or from receptors or their ligands. Antigen binding by the CARs triggers phosphorylation of immunoreceptor tyrosine-based activation motifs (ITAMs) in the intracellular domain, initiating a signaling cascade required for cytolysis induction, cytokine secretion, and proliferation.

In some embodiments, the CAR can be a “first generation,” “second generation” or “third generation” CAR (see, for example, Sadelain et al., Cancer Discov. 3(4):388-398 (2013); Jensen et al., Immunol. Rev. 257:127-133 (2014); Sharpe et al., Dis. Model Mech. 8(4):337-350 (2015); Brentjens et al., Clin. Cancer Res. 13:5426-5435 (2007); Gade et al., Cancer Res. 65:9080-9088 (2005); Maher et al., Nat. Biotechnol. 20:70-75 (2002); Kershaw et al., J. Immunol. 173:2143-2150 (2004); Sadelain et al., Curr. Opin. Immunol. 21(2):215-223 (2009); Hollyman et al., J Immunother. 32:169-180 (2009)).

“First generation” CARs are typically composed of an extracellular antigen binding domain, for example, a single-chain variable fragment (scFv), fused to a transmembrane domain, which is fused to a cytoplasmic/intracellular domain of the T cell receptor chain. “First generation” CARs typically have the intracellular domain from the CD3Q-chain, which is the primary transmitter of signals from endogenous T cell receptors (TCRs). “First generation” CARs can provide de novo antigen recognition and cause activation of both CD4⁺ and CD8⁺ T cells through their CD3ζ chain signaling domain in a single fusion molecule, independent of HLA-mediated antigen presentation. “Second-generation” CARs comprises a cancer antigen-binding domain fused to an intracellular signaling domain capable of activating immune cells such as T cells and a co-stimulatory domain designed to augment immune cell, such as T cell, potency and persistence (Sadelain et al., Cancer Discov. 3:388-398 (2013)). CAR design can therefore combine antigen recognition with signal transduction, two functions that are physiologically borne by two separate complexes, the TCR heterodimer and the CD3 complex. “Second generation” CARs include an intracellular domain from various co-stimulatory molecules, for example, CD28, 4-1BB, ICOS, OX40, and the like, in the cytoplasmic tail of the CAR to provide additional signals to the cell. “Second generation” CARs provide both co-stimulation, for example, by CD28 or 4-1BB domains, and activation, for example, by a CD3ζ signaling domain. Studies have indicated that “Second Generation” CARs can improve the anti-tumor activity of T cells. “Third generation” CARs provide multiple co-stimulation, for example, by comprising both CD28 and 4-1BB domains, and activation, for example, by comprising a CD3ζ activation domain. “Fourth generation” of CARs is based on second-generation CARs, but includes a protein, such as interleukin 12 (IL-12) that is constitutively or inducibly expressed upon CAR activation. T cells transduced with these fourth-generation CARs are referred to as T cells redirected for universal cytokine-mediated killing (TRUCKs). Activation of these CARs promotes the production and secretion of the desired cytokine to promote tumour killing though several synergistic mechanisms such as exocytosis (perform, granzyme) or death ligand-death receptor (Fas-FasL, TRAIL) systems. Additionally, “fifth generation” of CARs is currently being explored; these are based on the second generation of CARs, but they contain a truncated cytoplasmic IL-2 receptor $-chain domain with a binding site for the transcription factor STAT3. The antigen-specific activation of this receptor simultaneously triggers TCR (through the CD3ζ domains), co-stimulatory (CD28 domain) and cytokine (JAK-STAT3/5) signaling, which effectively provides all three synergistic signals required physiologically to drive full T cell activation and proliferation. Additional variants of the aforementioned CARs, such as dual CARs, split CARs and inducible-split CARs, have been generated to further enhance the specificity and control of the transfused T cells (Tokarew et al., British journal of cancer, 120.1 (2019): 26-37).

As described above, a CAR also contains a signaling domain that functions in the immune cell expressing the CAR. Such a signaling domain can be, for example, derived from CDζ or Fc receptor γ (see Sadelain et al., Cancer Discov. 3:388-398 (2013)). In general, the signaling domain will induce persistence, trafficking and/or effector functions in the transduced immune cells such as T cells (Sharpe et al., Dis. Model Mech. 8:337-350 (2015); Finney et al., J. Immunol. 161:2791-2797 (1998); Krause et al., J. Exp. Med. 188:619-626 (1998)). In the case of CDζ or Fc receptor γ, the signaling domain corresponds to the intracellular domain of the respective polypeptides, or a fragment of the intracellular domain that is sufficient for signaling. Exemplary signaling domains are described below in more detail.

CD3ζ. In a non-limiting embodiment, a CAR can comprise a signaling domain derived from a CD3ζ polypeptide, for example, a signaling domain derived from the intracellular domain of CD3ζ, which can activate or stimulate an immune cell, for example, a T cell. CD3ζ comprises 3 Immune-receptor-Tyrosine-based-Activation-Motifs (ITAMs), and transmits an activation signal to the cell, for example, a cell of the lymphoid lineage such as a T cell, after antigen is bound. A CD3ζ polypeptide can have an amino acid sequence corresponding to the sequence having GenBank No. NP_932170 (NP_932170.1, GJ:37595565; see below), or fragments thereof. In one embodiment, the CD3ζ polypeptide has an amino acid sequence of amino acids 52 to 164 of the CD3ζ polypeptide sequence provided below, or a fragment thereof that is sufficient for signaling activity. An exemplary CAR has an intracellular domain comprising a CD3ζ polypeptide comprising amino acids 52 to 164 of the CD3ζ polypeptide sequence provided below. Another exemplary CAR has an intracellular domain comprising a CD3ζ polypeptide comprising amino acids 52 to 164 of the CD3ζ polypeptide provided below. Still another exemplary CAR has an intracellular domain comprising a CD3ζ polypeptide comprising amino acids 52 to 164 of the CD3ζ polypeptide provided below. See GenBank NP_932170 for reference to domains within CD3ζ, for example, signal peptide, amino acids 1 to 21; extracellular domain, amino acids 22 to 30; transmembrane domain, amino acids 31 to 51; intracellular domain, amino acids 52 to 164.

(NP_932170; SEQ ID NO: 27)

| | |
|---|---|
| 1 | MKWKALFTAA ILQAQLPITE AQSFGLLDPK LCYLLDGILF |
| | IYGVILTALF LRVKFSRSAD |
| 61 | APAYQQGONQ LYNELNLGRR EEYDVLDKRR GRDPEMGGKP |
| | QRRKNPQEGL YNELQKDKMA |
| 121 | EAYSEIGMKG ERRRGKGHDG LYQGLSTATK DTYDALHMQA |
| | LPPR |

In certain non-limiting embodiments, an intracellular domain of a CAR can further comprise at least one co-stimulatory signaling domain. In some embodiments, an intracellular domain of a CAR can comprise two co-stimulatory signaling domains. Such a co-stimulatory signaling domain can provide increased activation of an immune cell. A co-stimulatory signaling domain can be derived from a CD28 polypeptide, a 4-1BB polypeptide, an OX40 polypeptide, an ICOS polypeptide, a DAP10 polypeptide, a 2B4 polypeptide, a CD27 polypeptide, a CD30 polypeptide, a CD40 polypeptide and the like. CARs comprising an intracellular domain that comprises a co-stimulatory signaling region comprising 4-1BB, ICOS or DAP-10 have been described previously (see U.S. Pat. No. 7,446,190, which is incorporated herein by reference, which also describes representative sequences for 4-1BB, ICOS and DAP-10). In some embodiments, the intracellular domain of a CAR can comprise a co-stimulatory signaling region that comprises two co-stimulatory molecules, such as CD28 and 4-1BB (see Sadelain et al., Cancer Discov. 3(4):388-398 (2013)), or CD28 and OX40, or other combinations of co-stimulatory ligands, as disclosed herein.

CD28. Cluster of Differentiation 28 (CD28) is a protein expressed on T cells that provides co-stimulatory signals for T cell activation and survival. CD28 is the receptor for CD80 (B7.1) and CD86 (B7.2) proteins. In one embodiment, a CAR can comprise a co-stimulatory signaling domain derived from CD28. For example, as disclosed herein, a CAR can include at least a portion of an intracellular/cytoplasmic domain of CD28, for example an intracellular/cytoplasmic domain that can function as a co-stimulatory signaling domain. A CD28 polypeptide can have an amino acid sequence corresponding to the sequence having GenBank No. P10747 (P10747.1, GI:115973) or NP_006130 (NP_006130.1, GJ:5453611), as provided below, or fragments thereof. If desired, CD28 sequences additional to the intracellular domain can be included in a CAR of the invention. For example, a CAR can comprise the transmembrane of a CD28 polypeptide. In one embodiment, a CAR can have an amino acid sequence comprising the intracellular domain of CD28 corresponding to amino acids 180 to 220 of CD28, or a fragment thereof. In another embodiment, a CAR can have an amino acid sequence comprising the transmembrane domain of CD28 corresponding to amino acids 153 to 179, or a fragment thereof. An exemplary CAR can comprise a co-stimulatory signaling domain corresponding to an intracellular domain of CD28. An exemplary CAR can also comprise a transmembrane domain derived from CD28. Thus, an exemplary CAR can comprise two domains from CD28, a co-stimulatory signaling domain and a transmembrane domain. In one embodiment, a CAR has an amino acid sequence comprising the transmembrane domain and the intracellular domain of CD28 and comprises amino acids 153 to 220 of CD28. In another embodiment, a CAR comprises amino acids 117 to 220 of CD28. Another exemplary CAR can comprise a transmembrane domain and intracellular domain of CD28. In one embodiment, a CAR can comprise a transmembrane domain derived from a CD28 polypeptide comprising amino acids 153 to 179 of the CD28 polypeptide provided below. See GenBank NP_006130 for reference to domains within CD28, for example, signal peptide, amino acids 1 to 18; extracellular domain, amino acids 19 to 152; transmembrane domain, amino acids 153 to 179; intracellular domain, amino acids 180 to 220. It is understood that sequences of CD28 that are shorter or longer than a specific delineated domain can be included in a CAR, if desired.

(NP_006130; SEQ ID NO: 28)

| | |
|---|---|
| 1 | MLRLLLALNL FPSIQVTGNK ILVKQSPMLV AYDNAVNLSC |
| | KYSYNLESRE FRASLHKGLD |
| 61 | SAVEVCVVYG NYSQQLQVYS KTGENCDGKL GNESVTFYLQ |
| | NLYVNQTDIY FCKIEVMYPP |
| 121 | PYLDNEKSNG TIIHVKGKHL CPSPLFPGPS KPFWVLVVVG |
| | GVLACYSLLV TVAFIIFWVR |
| 181 | SKRSRLLHSD YMNMTPRRPG PTRKHYQPYA PPRDFAAYRS |

4-1BB. 4-1BB, also referred to as tumor necrosis factor receptor superfamily member 9, can act as a tumor necrosis factor (TNF) ligand and have stimulatory activity. In one embodiment, a CAR can comprise a co-stimulatory signaling domain derived from 4-1BB. A 4-1BB polypeptide can have an amino acid sequence corresponding to the sequence having GenBank No. P41273 (P41273.1, GI:728739) or NP_001552 (NP_001552.2, GI:5730095) or fragments thereof. In one embodiment, a CAR can have a co-stimulatory domain comprising the intracellular domain of 4-1BB corresponding to amino acids 214 to 255, or a fragment thereof. In another embodiment, a CAR can have a transmembrane domain of 4-1BB corresponding to amino acids 187 to 213, or a fragment thereof. An exemplary CAR can have an intracellular domain comprising a 4-1BB polypeptide (for example, amino acids 214 to 255 of NP_001552) as provided below. See GenBank NP_001552 for reference to domains within 4-1BB, for example, signal peptide, amino acids 1 to 17; extracellular domain, amino acids 18 to 186; transmembrane domain, amino acids 187 to 213; intracellular domain, amino acids 214 to 255. It is understood that sequences of 4-1BB that are shorter or longer than a specific delineated domain can be included in a CAR, if desired.

(NP_001552; SEQ ID NO: 29)

| | |
|---|---|
| 1 | MGNSCYNIVA TLLLVLNFER TRSLQDPCSN CPAGTFCDNN |
| | RNQICSPCPP NSESSAGGQR |
| 61 | TCDICRQCKG VFRTRKECSS TSNAECDCTP GFHCLGAGCS |
| | MCEQDCKQGQ ELTKKGCKDC |
| 121 | CFGTENDQKR GICRPWTNCS LDGKSVLVNG TKERDVVCGP |
| | SPADLSPGAS SVTPPAPARE |
| 181 | PGHSPQIISF FLALTSTALL FLLFFLTLRF SVVKRGRKKL |
| | LYIFKQPFMR PVQTTQEEDG |
| 241 | CSCRFPEEEE GGCEL |

OX40. OX40, also referred to as tumor necrosis factor receptor superfamily member 4 precursor or CD134, is a member of the TNFR-superfamily of receptors. In one embodiment, a CAR can comprise a co-stimulatory signaling domain derived from OX40. An OX40 polypeptide can have an amino acid sequence corresponding to the sequence having GenBank No. P43489 (P43489.1, GI:1171933) or NP_003318 (NP_003318.1, GI:4507579), provided below, or fragments thereof. In one embodiment, a CAR can have a co-stimulatory domain comprising the intracellular domain of OX40 corresponding to amino acids 236 to 277, or a fragment thereof. In another embodiment, a CAR can have an amino acid sequence comprising the transmembrane domain of OX40 corresponding to amino acids 215 to 235 of OX40, or a fragment thereof. See GenBank NP_003318 for reference to domains within OX40, for example, signal peptide, amino acids 1 to 28; extracellular domain, amino acids 29 to 214; transmembrane domain, amino acids 215 to 235; intracellular domain, amino acids 236 to 277. It is understood that sequences of OX40 that are shorter or longer than a specific delineated domain can be included in a CAR, if desired.

(NP_003318; SEQ ID NO: 30)

| | |
|---|---|
| 1 | MCVGARRIGR GPCAALLLLG LGLSTVTGLH CVGDTYPSND |
| | RCCHECRPGN GMVSRCSRSQ |
| 61 | NTVCRPCGPG FYNDVVSSKP CKPCTWCNLR SGSERKQLCT |
| | ATQDTVCRCR AGTQPLDSYK |
| 121 | PGVDCAPCPP GHFSPGDNQA CKPWTNCTLA GKHTLQPASN |
| | SSDAICEDRD PPATQPQETQ |
| 181 | GPPARPITVQ PTEAWPRTSQ GPSTRPVEVP GGRAVAAILG |
| | LGLVLGLLGP LAILLALYLL |
| 241 | RRDQRLPPDA HKPPGGGSFR TPIQEEQADA HSTLAKI |

ICOS. Inducible T-cell costimulator precursor (ICOS), also referred to as CD278, is a CD28-superfamily costimulatory molecule that is expressed on activated T cells. In one embodiment, a CAR can comprise a co-stimulatory signaling domain derived from ICOS. An ICOS polypeptide can have an amino acid sequence corresponding to the sequence having GenBank No. NP_036224 (NP_036224.1, GI:15029518), provided below, or fragments thereof. In one embodiment, a CAR can have a co-stimulatory domain comprising the intracellular domain of ICOS corresponding to amino acids 162 to 199 of ICOS. In another embodiment, a CAR can have an amino acid sequence comprising the transmembrane domain of ICOS corresponding to amino acids 141 to 161 of ICOS, or a fragment thereof. See GenBank NP_036224 for reference to domains within ICOS, for example, signal peptide, amino acids 1 to 20; extracellular domain, amino acids 21 to 140; transmembrane domain, amino acids 141 to 161; intracellular domain, amino acids 162 to 199. It is understood that sequences of ICOS that are shorter or longer than a specific delineated domain can be included in a CAR, if desired.

(NP_036224; SEQ ID NO: 31)

| | |
|---|---|
| 1 | MKSGLWYFFL FCLRIKVLTG EINGSANYEM FIFHNGGVQI |
| | LCKYPDIVQQ FKMQLLKGGQ |
| 61 | ILCDLTKTKG SGNTVSIKSL KFCHSQLSNN SVSFFLYNLD |
| | HSHANYYFCN LSIFDPPPFK |
| 121 | VTLTGGYLHI YESQLCCQLK FWLPIGCAAF VVVCILGCIL |
| | ICWLTKKKYS SSVHDPNGEY |
| 181 | MFMRAVNTAK KSRLTDVTL |

DAP10. DAP10, also referred to as hematopoietic cell signal transducer, is a signaling subunit that associates with a large family of receptors in hematopoietic cells. In one embodiment, a CAR can comprise a co-stimulatory domain derived from DAP10. A DAP10 polypeptide can have an amino acid sequence corresponding to the sequence having GenBank No. NP_055081.1 (GI:15826850), provided below, or fragments thereof. In one embodiment, a CAR can have a co-stimulatory domain comprising the intracellular domain of DAP10 corresponding to amino acids 70 to 93, or a fragment thereof. In another embodiment, a CAR can have a transmembrane domain of DAP10 corresponding to amino acids 49 to 69, or a fragment thereof. See GenBank NP_055081.1 for reference to domains within DAP10, for example, signal peptide, amino acids 1 to 19; extracellular domain, amino acids 20 to 48; transmembrane domain, amino acids 49 to 69; intracellular domain, amino acids 70 to 93. It is understood that sequences of DAP10 that are shorter or longer than a specific delineated domain can be included in a CAR, if desired.

(SEQ ID NO: 32)

| | |
|---|---|
| 1 | MIHLGHILFL LLLPVAAAQT TPGERSSLPA FYPGTSGSCS |
| | GCGSLSLPLL AGLVAADAVA |
| 61 | SLLIVGAVEL CARPRRSPAQ EDGKVYINMP GRG |

CD27: CD27 (TNFRSF7) is a transmembrane receptor expressed on subsets of human CD8+ and CD4+ T-cells, NKT cells, NK cell subsets and hematopoietic progenitors and induced in FOXP3+CD4 T-cells and B cell subsets. Previously studies have found that CD27 can either actively provide costimulatory signals that improve human T-cell survival and anti-tumor activity in vivo. See Song and Powell; Oncoimmunology 1, no. 4 (2012): 547-549. In one embodiment, a CAR can comprise a co-stimulatory domain derived from CD27. A CD27 polypeptide can have an amino acid sequence corresponding to the sequence having UniProtKB/Swiss-Prot No.: P26842.2 (GI: 269849546), provided below, or fragments thereof. In one embodiment, a CAR can have a co-stimulatory domain comprising the intracellular domain of CD27 or a fragment thereof. In another embodiment, a CAR can have a transmembrane domain of CD27 or a fragment thereof. It is understood that sequences of CD27 that are shorter or longer than a specific delineated domain can be included in a CAR, if desired.

(SEQ ID NO: 33)

| | |
|---|---|
| 1 | MARPHPWWLC VLGTLVGLSA TPAPKSCPER HYWAQGKLCC |
| | QMCEPGTFLV KDCDOHRKAA |
| 61 | QCDPCIPGVS FSPDHHTRPH CESCRHCNSG LLVRNCTITA |
| | NAECACRNGW QCRDKECTEC |
| 121 | DPLPNPSLTA RSSQALSPHP QPTHLPYVSE MLEARTAGHM |
| | QTLADFROLP ARTLSTHWPP |
| 181 | QRSLCSSDFI RILVIFSGMF LVFTLAGALF LHQRRKYRSN |
| | KGESPVEPAE PCHYSCPREE |
| 241 | EGSTIPIQED YRKPEPACSP |

CD30: CD30 and its ligand (CD30L) are members of the tumor necrosis factor receptor (TNFR) and tumor necrosis factor (TNF) superfamilies, respectively. CD30, in many respects, behaves similarly to OX40 and enhances proliferation and cytokine production induced by TCR stimulation. Goronzy and Weyand, Arthritis research & therapy 10, no. S1 (2008): S3. In one embodiment, a CAR can comprise a co-stimulatory domain derived from CD30. A CD30 polypeptide can have an amino acid sequence corresponding to the sequence having GenBank No.: AAA51947.1 (GI: 180096), provided below, or fragments thereof. In one embodiment, a CAR can have a co-stimulatory domain comprising the intracellular domain of CD30 or a fragment thereof. In another embodiment, a CAR can have a transmembrane domain of CD30 or a fragment thereof. It is understood that sequences of CD30 that are shorter or longer than a specific delineated domain can be included in a CAR, if desired.

(SEQ ID NO: 34)

| | |
|---|---|
| 1 | MRVLLAALGL LFLGALRAFP QDRPFEDTCH GNPSHYYDKA |
| | VRRCCYRCPM GLFPTQQCPQ |
| 61 | RPTDCRKQCE PDYYLDEADR CTACVTCSRD DLVEKTPCAW |
| | NSSRVCECRP GMFCSTSAVN |
| 121 | SCARCFFHSV CPAGMIVKFP GTAQKNTVCE PASPGVSPAC |
| | ASPENCKEPS SGTIPQAKPT |
| 181 | PVSPATSSAS TMPVRGGTRL AQEAASKLTR APDSPSSVGR |
| | PSSDPGLSPT QPCPEGSGDC |
| 241 | RKQCEPDYYL DEAGRCTACV SCSRDDLVEK TPCAWNSSRT |
| | CECRPGMICA TSATNSCARC |
| 301 | VPYPICAAET VTKPQDMAEK DTTFEAPPLG TQPDCNPTPE |
| | NGEAPASTSP TQSLLVDSQA |
| 361 | SKTLPIPTSA PVALSSTGKP VLDAGPVLFW VILVLVVVVG |
| | SSAFLLCHRR ACRKRIRQKL |
| 421 | HLCYPVQTSQ PKLELVDSRP RRSSTQLRSG ASVTEPVAEE |
| | RGLMSQPLME TCHSVGAAYL |
| 481 | ESLPLQDASP AGGPSSPRDL PEPRVSTEHT NNKIEKIYIM |
| | KADTVIVGTV KAELPEGRGL |
| 541 | AGPAEPELEE ELEADHTPHY PEQETEPPLG SCSDVMLSVE |
| | EEGKEDPLPT AASGK |

CD40: CD40 and its ligand, CD40L or CD154, were first identified as instrumental in T-cell-dependent B-cell activation. The pathway is now recognized as a mechanism to activate APCs and to enhance their potential to activate T cells. CD154-mediated CD40 stimulation provides an important feedback mechanism for the initial co-stimulatory pathway of CD28-CD80/CD86. Goronzy and Weyand, Arthritis research & therapy 10, no. S1 (2008): S3. In one embodiment, a CAR can comprise a co-stimulatory domain derived from CD40. A CD40 polypeptide can have an amino acid sequence corresponding to the sequence having UniProtKB/Swiss-Prot No.: P25942.1 (GI: 269849546), provided below, or fragments thereof. In one embodiment, a CAR can have a co-stimulatory domain comprising the intracellular domain of CD40 or a fragment thereof. In another embodiment, a CAR can have a transmembrane domain of CD40 or a fragment thereof. It is understood that sequences of CD40 that are shorter or longer than a specific delineated domain can be included in a CAR, if desired.

(SEQ ID NO: 35)

| | |
|---|---|
| 1 | MVRLPLQCVL WGCLLTAVHP EPPTACREKQ YLINSQCCSL |
| | CQPGQKLVSD CTEFTETECL |
| 61 | PCGESEFLDT WNRETHCHQH KYCDPNLGLR VQQKGTSETD |
| | TICTCEEGWH CTSEACESCV |
| 121 | LHRSCSPGFG VKQIATGVSD TICEPCPVGF FSNVSSAFEK |
| | CHPWTSCETK DLVVQQAGTN |
| 181 | KTDVVCGPQD RLRALVVIPI IFGILFAILL VLVFIKKVAK |
| | KPTNKAPHPK QEPQEINFPD |
| 241 | DLPGSNTAAP VQETLHGCQP VTQEDGKESR ISVQERQ |

The extracellular domain of a CAR can be fused to a leader or a signal peptide that directs the nascent protein into the endoplasmic reticulum and subsequent translocation to the cell surface. It is understood that, once a polypeptide containing a signal peptide is expressed at the cell surface, the signal peptide has generally been proteolytically removed during processing of the polypeptide in the endoplasmic reticulum and translocation to the cell surface. Thus, a polypeptide such as a CAR is generally expressed at the cell surface as a mature protein lacking the signal peptide, whereas the precursor form of the polypeptide includes the signal peptide. A signal peptide or leader can be essential if a CAR is to be glycosylated and/or anchored in the cell membrane. The signal sequence or leader is a peptide sequence generally present at the N-terminus of newly synthesized proteins that directs their entry into the secretory pathway. The signal peptide is covalently joined to the N-terminus of the extracellular antigen-binding domain of a CAR as a fusion protein. In one embodiment, the signal peptide comprises a CD8 polypeptide comprising amino acids MALPVTALLLPLALLLHAARP (SEQ ID NO:36). It is understood that use of a CD8 signal peptide is exemplary. Any suitable signal peptide, as are well known in the art, can be applied to a CAR to provide cell surface expression in an immune cell (see Gierasch Biochem. 28:923-930 (1989); von Heijne, J. Mol. Biol. 184 (1):99-10⁵ (1985)). Particularly useful signal peptides can be derived from cell surface proteins naturally expressed in the immune cell provided herein, including any of the signal peptides of the polypeptides disclosed herein. Thus, any suitable signal peptide can be utilized to direct a CAR to be expressed at the cell surface of an immune cell provided herein.

In certain non-limiting embodiments, an extracellular antigen-binding domain of a CAR can comprise a linker sequence or peptide linker connecting the heavy chain variable region and light chain variable region of the extracellular antigen-binding domain. In one non-limiting example, the linker comprises amino acids having the sequence set forth in GGGGSGGGGSGGGGS (SEQ ID NO:37).

In certain non-limiting embodiments, a CAR can also comprise a spacer region or sequence that links the domains of the CAR to each other. For example, a spacer can be included between a signal peptide and an antigen binding domain, between the antigen binding domain and the transmembrane domain, between the transmembrane domain and the intracellular domain, and/or between domains within the intracellular domain, for example, between a stimulatory domain and a co-stimulatory domain. The spacer region can be flexible enough to allow interactions of various domains with other polypeptides, for example, to allow the antigen binding domain to have flexibility in orientation in order to facilitate antigen recognition. The spacer region can be, for example, the hinge region from an IgG, the CH₂CH₃ (constant) region of an immunoglobulin, and/or portions of CD3 (cluster of differentiation 3) or some other sequence suitable as a spacer.

The transmembrane domain of a CAR generally comprises a hydrophobic alpha helix that spans at least a portion of the membrane. Different transmembrane domains result in different receptor stability. After antigen recognition, receptors cluster and a signal is transmitted to the cell. In an embodiment, the transmembrane domain of a CAR can be derived from another polypeptide that is naturally expressed in the immune cell. In one embodiment, a CAR can have a transmembrane domain derived from CD8, CD28, CD3ζ, CD4, 4-1BB, OX40, ICOS, CTLA-4, PD-1, LAG-3, 2B4, BTLA, or other polypeptides expressed in the immune cell. Optionally, the transmembrane domain can be derived from a polypeptide that is not naturally expressed in the immune cell, so long as the transmembrane domain can function in transducing signal from antigen bound to the CAR to the intracellular signaling and/or co-stimulatory domains. It is understood that the portion of the polypeptide that comprises a transmembrane domain of the polypeptide can include additional sequences from the polypeptide, for example, additional sequences adjacent on the N-terminal or C-terminal end of the transmembrane domain, or other regions of the polypeptide, as desired.

CD8. Cluster of differentiation 8 (CD8) is a transmembrane glycoprotein that serves as a co-receptor for the T cell receptor (TCR). CD8 binds to a major histocompatibility complex (MHC) molecule and is specific for the class I MHC protein. In one embodiment, a CAR can comprise a transmembrane domain derived from CD8. A CD8 polypeptide can have an amino acid sequence corresponding to the sequence having GenBank No. NP_001139345.1 (GI:225007536), as provided below, or fragments thereof. In one embodiment, a CAR can have an amino acid sequence comprising the transmembrane domain of CD8 corresponding to amino acids 183 to 203, or fragments thereof. In one embodiment, an exemplary CAR has a transmembrane domain derived from a CD8 polypeptide. In one non-limiting embodiment, a CAR can comprise a transmembrane domain derived from a CD8 polypeptide comprising amino acids 183 to 203. In addition, a CAR can comprise a hinge domain comprising amino acids 137-182 of the CD8 polypeptide provided below. In another embodiment, a CAR can comprise amino acids 137-203 of the CD8 polypeptide provided below. In yet another embodiment, a CAR can comprise amino acids 137 to 209 of the CD8 polypeptide provided below. See GenBank NP_001139345.1 for reference to domains within CD8, for example, signal peptide, amino acids 1 to 21; extracellular domain, amino acids 22 to 182; transmembrane domain amino acids, 183 to 203; intracellular domain, amino acids 204 to 235. It is understood that additional sequence of CD8 beyond the transmembrane domain of amino acids 183 to 203 can be included in a CAR, if desired. It is further understood that sequences of CD8 that are shorter or longer than a specific delineated domain can be included in a CAR, if desired.

(NP_001139345.1; SEQ ID NO: 38)

| | |
|---|---|
| 1 | MALPVTALLL PLALLLHAAR PSQFRVSPLD RTWNLGETVE |
| | LKCQVLLSNP TSGCSWLFQP |
| 61 | RGAAASPTFL LYLSQNKPKA AEGLDTORFS GKRLGDTFVL |
| | TLSDERRENE GYYFCSALSN |
| 121 | SIMYFSHFVP VFLPAKPTTT PAPRPPTPAP TIASQPLSLR |
| | PEACRPAAGG AVHTRGLDFA |
| 181 | CDIYIWAPLA GTCGVLLLSL VITLYCNHRN RRRVCKCPRP |
| | VVKSGDKPSL SARYV |

CD4. Cluster of differentiation 4 (CD4), also referred to as T-cell surface glycoprotein CD4, is a glycoprotein found on the surface of immune cells such as T helper cells, monocytes, macrophages, and dendritic cells. In one embodiment, a CAR can comprise a transmembrane domain derived from CD4. CD4 exists in various isoforms. It is understood that any isoform can be selected to achieve a desired function. Exemplary isoforms include isoform 1 (NP_000607.1, GI:10835167), isoform 2 (NP_001181943.1, GI:303522479), isoform 3 (NP_001181944.1, GI:303522485; or NP_001181945.1, GI:303522491; or NP_001181946.1, GI:303522569), and the like. One exemplary isoform sequence, isoform 1, is provided below. In one embodiment, a CAR can have an amino acid sequence comprising the transmembrane domain of CD4 corresponding to amino acids 397 to 418, or fragments thereof. See GenBank NP_000607.1 for reference to domains within CD4, for example, signal peptide, amino acids 1 to 25; extracellular domain, amino acids 26 to 396; transmembrane domain amino acids, 397 to 418; intracellular domain, amino acids 419 to 458. It is understood that additional sequence of CD4 beyond the transmembrane domain of amino acids 397 to 418 can be included in a CAR, if desired. It is further understood that sequences of CD4 that are shorter or longer than a specific delineated domain can be included in a CAR, if desired.

(NP_000607.1; SEQ ID NO: 39)

| | |
|---|---|
| 1 | MNRGVPFRHL LLVLQLALLP AATQGKKVVL GKKGDTVELT |
| | CTASQKKSIQ FHWKNSNQIK |
| 61 | ILGNQGSFLT KGPSKLNDRA DSRRSLWDQG NFPLIIKNLK |
| | IEDSDTYICE VEDQKEEVQL |
| 121 | LVFGLTANSD THLLQGQSLT LTLESPPGSS PSVQCRSPRG |
| | KNIQGGKTLS VSQLELQDSG |
| 181 | TWTCTVLQNQ KKVEFKIDIV VLAFQKASSI VYKKEGEQVE |
| | FSFPLAFTVE KLTGSGELWW |
| 241 | QAERASSSKS WITFDLKNKE VSVKRVTQDP KLQMGKKLPL |
| | HLTLPQALPQ YAGSGNLTLA |
| 301 | LEAKTGKLHQ EVNLVVMRAT QLQKNLTCEV WGPTSPKLML |
| | SLKLENKEAK VSKREKAVWV |
| 361 | LNPEAGMWQC LLSDSGQVLL ESNIKVLPTW STPVQPMALI |
| | VLGGVAGLLL FIGLGIFFCV |
| 421 | RCRHRRRQAE RMSQIKRLLS EKKTCQCPHR FQKTCSPI |

FcRγ Activating types of IgG receptor FcγRs form multimeric complexes including the Fc receptor common γ chain (FcRγ) that contains an intracellular tyrosine-based activating motif (ITAM), whose activation triggers oxidative bursts, cytokine release, phagocytosis, antibody-dependent cell-mediated cytotoxicity, and degranulation. In one embodiment, a CAR can comprise a transmembrane domain derived from FcRγ. In one embodiment, a CAR can comprise a co-stimulatory domain derived from FcRγ. An FcRγ polypeptide can have an amino acid sequence corresponding to the sequence having NCBI Reference Sequence: NP_004097.1 (GI: 4758344), provided below, or fragments thereof. In one embodiment, a CAR can have a co-stimulatory domain comprising the intracellular domain of FcRγ, or a fragment thereof. In another embodiment, a CAR can have a transmembrane domain of FcRγ, or a fragment thereof.

(SEQ ID NO: 40)

| | |
|---|---|
| 1 | MIPAVVLLLL LLVEQAAALG EPQLCYILDA ILFLYGIVLT |
| | LLYCRLKIQV RKAAITSYEK |
| 61 | SDGVYTGLST RNQETYETLK HEKPPQ |

CARs provided herein can include a targeting moiety as disclosed above. GdT cells provided herein can express CARs targeting a tumor antigen selected from the group consisting of CD19, CD20, CD22, CD30, CD123, CD138, CD33, CD70, BCMA, CS1, C-Met, IL13Ra2, EGFRvIII, CEA, Her2, GD2, MAGE, GPC3, Mesothelin, PSMA, ROR1, EGFR, MUC1, and NY-ESO-1 in a cell. In some embodiments, gdT cells provided herein can express CARs having an antibody or antigen-binding unit that target a tumor antigen selected from the group consisting of CD19, CD20, CD22, CD30, CD123, CD138, CD33, CD70, BCMA, CS1, C-Met, IL13Ra2, EGFRvIII, CEA, Her2, GD2, MAGE, GPC3, Mesothelin, PSMA, ROR1, EGFR, MUC1, and NY-ESO-1.

For exemplary purposes, in some embodiments, the CAR comprises an anti-CD19 antibody as the targeting moiety. In some embodiments, the CAR comprises an anti-BCMA antibody as the targeting moiety. In some embodiments, the CAR comprises an anti-CD22 antibody as the targeting moiety. In some embodiments, the CAR comprises an anti-CD20 antibody (e.g., rituximab). In some embodiments, the CAR comprises an anti-HER2 antibody (e.g., trastuzumab).

### 5.2.3.2 TCRs

In some embodiments, the targeting moiety is exogenously expressed on the cell surface as part of a receptor protein. In some embodiments, the receptor protein is a TCR. TCRs are antigen-specific molecules that are responsible for recognizing antigenic peptides presented in the context of a product of the MHC on the surface of antigen presenting cells (APCs) or any nucleated cells. This system endows T cells, via their TCRs, with the potential ability to recognize the entire array of intracellular antigens expressed by a cell (including virus proteins) that are processed into short peptides, bound to an intracellular MHC molecule, and delivered to the surface as a peptide-MHC complex. This system allows foreign protein (e.g., mutated cancer antigen or virus protein) or aberrantly expressed protein to serve a target for T cells (e.g., Davis and Bjorkman (1988) Nature, 334, 395-402; Davis et al., (1998) Annu Rev Immunol, 16, 523-544).

The interaction of a TCR and a peptide-MHC complex can drive the T cell into various states of activation, depending on the affinity (or dissociation rate) of binding. The TCR recognition process allows a T cell to discriminate between a normal, healthy cell and, for example, one that has become transformed via a virus or malignancy, by providing a diverse repertoire of TCRs, wherein there is a high probability that one or more TCRs will be present with a binding affinity for the foreign peptide bound to an MHC molecule that is above the threshold for stimulating T cell activity (Manning and Kranz (1999) Immunology Today, 20, 417-422).

Wild type TCRs isolated from either human or mouse T cell clones that were identified by in vitro culturing have been shown to have relatively low binding affinities (K_{D}=1-300 μM) (Davis et al. (1998) Annu Rev Immunol, 16, 523-544). This is partly because that T cells that develop in the thymus are negatively selected (tolerance induction) on self-peptide-MHC ligands, such that T cells with too high of an affinity are deleted (Starr et al. (2003) Annu Rev Immunol, 21, 139-76). To compensate for these relatively low affinities, T cells have evolved a co-receptor system in which the cell surface molecules CD4 and CD8 bind to the MHC molecules (class II and class I, respectively) and synergize with the TCR in mediating signaling activity. CD8 is particularly effective in this process, allowing TCRs with very low affinity (e.g., K_{D}=300 μM) to mediate potent antigen-specific activity.

Directed evolution can be used to generate TCRs with higher affinity for a specific peptide-MHC complex. Methods that can be used include yeast display (Holler et al. (2003) Nat Immunol, 4, 55-62; Holler et al. (2000) Proc Natl Acad Sci USA, 97, 5387-92), phage display (Li et al. (2005) Nat Biotechnol, 23, 349-54), and T cell display (Chervin et al. (2008) J Immunol Methods, 339, 175-84). All three approaches involve engineering, or modifying, a TCR that exhibits the normal, low affinity of the wild-type TCR, to increase the affinity for the cognate peptide-MHC complex (the original antigen that the T cells were specific for).

As such, in some embodiments, the gdT cells provided herein can exogenously express TCRs in cell surface. In some embodiments, the TCR comprises an alpha (α) chain and a beta (β) chain (encoded by TRAC and TRBC, respectively). A human TRAC can have an amino acid sequence corresponding to UniProtKB/Swiss-Prot No.: P01848.2 (Accession: P01848.2 GI: 1431906459). A human TRBC can have an amino acid sequence corresponding to the GenBank sequence ALC78509.1 (Accession: ALC78509.1 GI: 924924895). In some embodiments, the TCR comprises a gamma chain (γ) and a delta (δ) chain (encoded by TRGC and TRDC, respectively). A human TRGC can have an amino acid sequence corresponding to UniProtKB/Swiss-Prot: POCF51.1 (Accession: POCF51.1 GI: 294863156), or an amino acid sequence corresponding to UniProtKB/Swiss-Prot: P03986.2 (Accession: P03986.2 GI: 1531253869). A human TRDC can have an amino acid sequence corresponding to the UniProtKB/Swiss-Prot: B7Z8K6.2 (Accession: B7Z8K6.2 GI: 294863191). The extracellular regions of the αβ chains (or the γδ chains) are responsible for antigen recognition and engagement. Antigen binding stimulates downstream signaling through the multimeric CD3 complex that associates with the intracellular domains of the αβ (or γδ) chains as three dimers (εγ, εδ, ζζ).

TCRs provided herein can be genetically engineered to bind specific antigens. In some embodiments, gdT cells provided herein can express a TCR having a targeting moiety targeting a tumor antigen in a cell. In some embodiments, the tumor antigen is selected from the group consisting of CD19, CD20, CD22, CD30, CD123, CD138, CD33, CD70, BCMA, CS1, C-Met, IL13Ra2, EGFRvIII, CEA, Her2, GD2, MAGE, GPC3, Mesothelin, PSMA, ROR1, EGFR, MUC1, and NY-ESO-1. In some embodiments, the targeting moiety is an antibody or antigen-binding unit, and the gdT cells provided herein can express a TCR having an antibody or antigen-binding unit targeting a tumor antigen selected from the group consisting of CD19, CD20, CD22, CD30, CD123, CD138, CD33, CD70, BCMA, CS1, C-Met, IL13Ra2, EGFRvIII, CEA, Her2, GD2, MAGE, GPC3, Mesothelin, PSMA, ROR1, EGFR, MUC1, and NY-ESO-1.

For exemplary purposes, in some embodiments, the TCR comprises an anti-CD19 antibody as the targeting moiety. In some embodiments, the TCR comprises an anti-BCMA antibody as the targeting moiety. In some embodiments, the TCR comprises an anti-CD22 antibody as the targeting moiety. In some embodiments, the TCR comprises an anti-CD20 antibody (e.g., rituximab). In some embodiments, the TCR comprises an anti-HER2 antibody (e.g., trastuzumab).

5.2.3.3 Methods of Producing gdT Cells with CAR/TCR

With respect to modifying gdT cells provided herein to recombinantly express a CAR or TCR disclosed herein, one or more nucleic acids encoding the CAR or TCR can be introduced into the cells using a suitable expression vector (e.g., Rozenbaum et al., Frontiers in immunology 11 (2020): 1347). In some embodiments, provided herein are methods of manufacturing a cell population enriched in CAR gdT cells or TCR gdT cells having NK-like properties comprising the culturing methods described in Section 5.1 above, further comprising introducing a nucleic acid encoding a CAR or TCR to the gdT cells. As a person of ordinary skill in the art would understand, the nucleic acid encoding a CAR or TCR can be introduced at different times during the culture. In some embodiments, the nucleic acid is introduced in the beginning of the culture. In some embodiments, the nucleic acid is introduced toward the end of the culture. In some embodiments, the nucleic acid encoding a CAR or TCR can be introduced on Day 4, 5, 6, 7, 8, 9, 10, 11, 12, 13, 14, 15, 16, 17, 18, 19, 20, 21, 22, 23, 24, 25, 26, 27, 28, 29, or 30 of the culture. In some embodiments, the nucleic acid encoding a CAR or TCR is introduced after the gdT cells have expanded for a period of time (e.g., 1 to 10 days, 1 to 8 days, 1 to 6 days, 1 to 4 days, or 1 to 2 days). In some embodiments, the nucleic acid encoding a CAR or TCR can be introduced on Day 2 or later, Day 3 or later, Day 4 or later, Day 5 or later, or Day 6 or later. In some embodiments that involve depletion of abT cells, the nucleic acid can be introduced to the gdT cells before or after the depletion of abT cells. A person of ordinary skill in the art would be able to further optimize the procedures.

For illustrative purposes, provided below is an exemplary method of manufacturing, from PBMCs, a cell population enriched in CAR gdT cells having NK-like properties, which comprises culturing the cells for 16 days and includes the following procedures:

| Day | Procedure |
|---|---|
| 1-4 | Culturing the starting cell population in culturing medium |
| | supplemented with IL-2, HPL, and zoledronate, allowing initial |
| | proliferation of gdT cells |
| 5 | Transducing the cells with a CAR/TCR-encoding nucleic acid |
| 6-8 | Continuing the culture in culturing medium supplemented with |
| | IL-2, and HPL |
| 9 | Depleting abT cells from the cell population |
| 10-16 | Continuing the culture in culturing medium supplemented with |
| | IL-2 and HPL, allowing further proliferation of the transduced |
| | gdT cells before harvesting resulting cell population on Day 16 |

The target gdT cells can be introduced with one or more nucleic acids encoding a CAR or TCR. Physical methods for introducing a polynucleotide into a host cell include calcium phosphate precipitation, lipofection, particle bombardment, microinjection, electroporation, and the like. In some embodiments, DNA transfection and transposon can be used. In some embodiments, the Sleeping Beauty system or PiggyBac system is used (e.g., Ivics et al., Cell, 91 (4): 501-510 (1997); Cadinanos et al. (2007) Nucleic Acids Research. 35 (12): e87). Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In some embodiments, a nucleic acid encoding a CAR or TCR can be cloned into a suitable vector, such as a retroviral vector, and introduced into the target gdT cells cell using well known molecular biology techniques (see Ausubel et al., Current Protocols in Molecular Biology, John Wiley and Sons, Baltimore, MD (1999)). Any vector suitable for expression in a cell, particularly a human immune cell, can be used. The vectors contain suitable expression elements such as promoters that provide for expression of the encoded nucleic acids in the target cell. In the case of a retroviral vector, cells can optionally be activated to increase transduction efficiency (see Parente-Pereira et al., J Biol. Methods 1(2) e7 (doi 10.14440/jbm.2014.30) (2014); Movassagh et al., Hum. Gene Ther. 11:1189-1200 (2000); Rettig et al., Mol. Ther. 8:29-41 (2003); Agarwal et al., J. Virol. 72:3720-3728 (1998); Pollok et al., Hum. Gene Ther. 10:2221-2236 (1998); Quinn et al., Hum. Gene Ther. 9:1457-1467 (1998); see also commercially available methods such as Dynabeads™ human T cell activator products, Thermo Fisher Scientific, Waltham, MA).

In one embodiment, the vector is a retroviral vector, for example, a gamma retroviral or lentiviral vector, which is employed for the introduction of a CAR or TCR into the target cell. For genetic modification of the cells to express a CAR or TCR, a retroviral vector is generally employed for transduction. However, it is understood that any suitable viral vector or non-viral delivery system can be used. Combinations of a retroviral vector and an appropriate packaging line are also suitable, where the capsid proteins will be functional for infecting human cells. Various amphotropic virus-producing cell lines are known, including, but not limited to, PA12 (Miller et al., Mol. Cell. Biol. 5:431-437 (1985)); PA317 (Miller et al., Mol. Cell. Biol. 6:2895-2902(1986)); and CRIP (Danos et al., Proc. Natl. Acad. Sci. USA 85:6460-6464 (1988)). Non-amphotropic particles are suitable too, for example, particles pseudotyped with VSVG, RD114 or GALV envelope and any other known in the art (Relander et al., Mol. Therap. 11:452-459 (2005)). Possible methods of transduction also include direct co-culture of the cells with producer cells (for example, Bregni et al., Blood 80:1418-1422 (1992)), or culturing with viral supernatant alone or concentrated vector stocks with or without appropriate growth factors and polycations (see, for example, Xu et al., Exp. Hemat. 22:223-230 (1994); Hughes, et al. J. Clin. Invest. 89:1817-1824 (1992)).

Generally, the chosen vector exhibits high efficiency of infection and stable integration and expression (see, for example, Cayouette et al., Human Gene Therapy 8:423-430 (1997); Kido et al., Current Eye Research 15:833-844 (1996); Bloomer et al., J. Virol. 71:6641-6649 (1997); Naldini et al., Science 272:263 267 (1996); and Miyoshi et al., Proc. Natl. Acad. Sci. U.S.A. 94:10319-10323 (1997)). Other viral vectors that can be used include, for example, adenoviral, lentiviral, and adeno-associated viral vectors, vaccinia virus, a bovine papilloma virus derived vector, or a herpes virus, such as Epstein-Barr Virus (see, for example, Miller, Hum. Gene Ther. 1(1):5-14 (1990); Friedman, Science 244:1275-1281 (1989); Eglitis et al., BioTechniques 6:608-614 (1988); Tolstoshev et al., Current Opin. Biotechnol. 1:55-61 (1990); Sharp, Lancet 337:1277-1278 (1991); Cornetta et al., Prog. Nucleic Acid Res. Mol. Biol. 36:311-322 (1989); Anderson, Science 226:401-409 (1984); Moen, Blood Cells 17:407-416 (1991); Miller et al., Biotechnology 7:980-990 (1989); Le Gal La Salle et al., Science 259:988-990 (1993); and Johnson, Chest 107:77S-83S (1995)). Retroviral vectors are particularly well developed and have been used in clinical settings (Rosenberg et al., N. Engl. J. Med. 323:370 (1990); Anderson et al., U.S. Pat. No. 5,399,346).

Particularly useful vectors for expressing a fusion protein disclosed herein and/or synthetic receptor include vectors that have been used in human gene therapy. In one non-limiting embodiment, a vector is a retroviral vector. The use of retroviral vectors for expression in T cells or other immune cells, including engineered T cells, has been described (see Scholler et al., Sci. Transl. Med. 4:132-153 (2012; Parente-Pereira et al., J Biol. Methods 1(2):e7 (1-9)(2014); Lamers et al., Blood 117(1):72-82 (2011); Reviere et al., Proc. Natl. Acad. Sci. USA 92:6733-6737 (1995)). In one embodiment, the vector is an SGF retroviral vector such as an SGF γ-retroviral vector, which is Moloney murine leukemia-based retroviral vector. SGF vectors have been described previously (see, for example, Wang et al., Gene Therapy 15:1454-1459 (2008)).

The vectors used herein employ suitable promoters for expression in a particular host cell. The promoter can be an inducible promoter or a constitutive promoter. In a particular embodiment, the promoter of an expression vector provides expression in a stem cell, such as a hematopoietic stem cell. In a particular embodiment, the promoter of an expression vector provides expression in an immune cell, such as a T cell. Non-viral vectors can be used as well, so long as the vector contains suitable expression elements for expression in the target cell. Some vectors, such as retroviral vectors, can integrate into the host genome. If desired, targeted integration can be implemented using technologies such as a nuclease, transcription activator-like effector nucleases (TALENs), Zinc-finger nucleases (ZFNs), and/or clustered regularly interspaced short palindromic repeats (CRISPRs), homologous recombination, non-homologous end joining, microhomology-mediated end joining, homology-mediated end joining and the like (Gersbach et al., Nucl. Acids Res. 39:7868-7878 (2011); Vasileva, et al. Cell Death Dis. 6:e1831. (Jul. 23, 2015); Sontheimer, Hum. Gene Ther. 26(7):413-424 (2015); Yao et al. Cell Research volume 27, pages 801-814(2017)).

The vectors and constructs can optionally be designed to include a reporter. For example, the vector can be designed to express a reporter protein, which can be useful to identify cells comprising the vector or nucleic acids provided on the vector, such as nucleic acids that have integrated into the host chromosome. In one embodiment, the reporter can be expressed as a bicistronic or multicistronic expression construct with the fusion protein or synthetic receptor. Exemplary reporter proteins include, but are not limited to, fluorescent proteins, such as mCherry, green fluorescent protein (GFP), blue fluorescent protein, for example, EBFP, EBFP2, Azurite, and mKalama1, cyan fluorescent protein, for example, ECFP, Cerulean, and CyPet, and yellow fluorescent protein, for example, YFP, Citrine, Venus, and YPet.

Assays can be used to determine the transduction efficiency of a fusion protein disclosed herein or a synthetic receptor using routine molecular biology techniques. If a marker has been included in the construct, such as a fluorescent protein, gene transfer efficiency can be monitored by FACS analysis to quantify the fraction of transduced (for example, GFP*) immune cells, such as T cells, and/or by quantitative PCR. Using a well-established cocultivation system (Gade et al., Cancer Res. 65:9080-9088 (2005); Gong et al., Neoplasia 1:123-127 (1999); Latouche et al., Nat. Biotechnol. 18:405-409 (2000)) it can be determined whether fibroblast AAPCs expressing cancer antigen (vs. controls) direct cytokine release from transduced immune cells, such as T cells, expressing a synthetic receptor (e.g. CAR) (cell supernatant LUMINEX (Austin TX) assay for IL-2, IL-4, IL-10, IFN-γ, TNF-α, and GM-CSF), T cell proliferation (by carboxyfluorescein succinimidyl ester (CFSE) labeling), and T cell survival (by Annexin V staining). The influence of CD80 and/or 4-1BBL on T cell survival, proliferation, and efficacy can be evaluated. T cells can be exposed to repeated stimulation by cancer antigen positive target cells, and it can be determined whether T cell proliferation and cytokine response remain similar or diminished with repeated stimulation. The cancer antigen CAR constructs can be compared side by side under equivalent assay conditions. Cytotoxicity assays with multiple E:T ratios can be conducted using chromium-release assays.

### 5.2.4 Pharmaceutical Compositions

Provided herein are also pharmaceutical compositions comprising the gdT-enriched cell populations described herein and a pharmaceutically acceptable carrier. In some embodiments, the pharmaceutical compositions provided herein can further comprise one or more additional active agents, such as an active agent suitable for treating the diseases that the pharmaceutical compositions are intended for. For example, antibodies that specifically bind tumor antigens can stimulate or enhance an ADCC response from the cell populations described herein, and can therefore be used in combination with the cell populations or pharmaceutical compositions described herein.

The term “pharmaceutically acceptable carrier” or “pharmaceutically acceptable excipient” refers to a material that is suitable for drug administration to an individual along with an active agent without causing undesirable biological effects or interacting in a deleterious manner with any of the other components of the pharmaceutical composition.

In some embodiments, the pharmaceutical composition is an aqueous formulation. Such a formulation is typically a solution or a suspension, but can also include colloids, dispersions, emulsions, and multi-phase materials. The term “aqueous formulation” is defined as a formulation comprising at least 50% w/w water. Likewise, the term “aqueous solution” is defined as a solution comprising at least 50% w/w water, and the term “aqueous suspension” is defined as a suspension comprising at least 50% w/w water. Pharmaceutically acceptable carriers that can be used in pharmaceutical compositions provided herein include any and all solvents, dispersion media, coatings, antibacterial and antifungal agents, isotonic and absorption delaying agents, and the like that are physiologically compatible. Pharmaceutically acceptable carriers can include, for example, buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminium hydroxide); and preservatives. In some embodiments, the pharmaceutical compositions are cryopreserved, to which the physician or the patient adds solvents and/or diluents prior to use; and cryopreservation solutions which can be used in the pharmaceutical compositions described herein include, for example, DMSO.

In some embodiments, the pharmaceutical compositions provided herein are substantially free of contaminant. In some embodiments, the pharmaceutical compositions provided herein have no detectable levels of contaminants. The contaminants include, for example, endotoxin, mycoplasma, bacterial components, and feeder cells (e.g., transformed cells).

The cell populations in the pharmaceutical compositions provided herein are enriched in gdT cells. In some embodiments, the cell populations for use as a medicament comprise at least 50% gdT, such as more than 60%, more than 70%, more than 80%, more than 90%, more than 95% or more than 99% gdT cells. In some embodiments, the cell populations for use as a medicament comprise at least 80% gdT. In some embodiments, the cell populations for use as a medicament comprise at least 85% gdT. In some embodiments, the cell populations for use as a medicament comprise at least 90% gdT. In some embodiments, the cell populations for use as a medicament comprise at least 95% gdT. In some embodiments, the cell populations are enriched in CD69+gdT cells. In some embodiments, the cell populations for use as a medicament comprise at least 70% gdT cells, wherein (1) the gdT cells express at least 400 DNAM-1 molecules per cell on average; (2) at least 30% of the gdT cells are CD69⁺; or both (1) and (2). In some embodiments, the cell populations comprise at least 70% gdT cells, wherein (1) the gdT cells express at least 400 DNAM-1 molecules per cell on average and (2) at least 30% of the gdT cells are CD69+. In some embodiments, the gdT cells express at least 500, at least 1000, at least 2000, or at least 3000 DNAM-1 molecules per cell on average. In some embodiments, at least 40%, at least 50%, at least 55%, at least 60%, at least 65%, at least 70%, at least 75%, or at least 80% of the gdT cells are CD69+. In some embodiments of the cell populations for use as a medicament, at least 30%, at least 40%, at least 50%, at least 60%, at least 70%, or at least 80% of the gdT cells are TDEM cells.

Pharmaceutical compositions provided herein can be formulated, for example, for parenteral (e.g., intravenous, subcutaneous, intraperitoneal, intramuscular, intrathecal) administration. In some embodiments, the pharmaceutical compositions provided herein are formulated for parenteral administration. In some embodiments, the carriers included in the pharmaceutical compositions provided herein are suitable for parenteral administration (e.g., by injection or infusion). In some embodiments, the pharmaceutical compositions provided herein are formulated for intravenous administration. In some embodiments, the carriers included in the pharmaceutical compositions provided herein are suitable for intravenous administration.

The pharmaceutical compositions provided herein can be stored at or below 0° C. In some embodiments, the cell populations or pharmaceutical compositions provided herein can maintain their therapeutic potency when stored at or below 0° C. for at least one week, at least two weeks, at least 1 month, at least 3 months, at least 6 months, or at least 1 year.

In some embodiments, the cell populations or pharmaceutical compositions provided herein are stored at or below 4° C., 0° C., or −20° C. In some embodiments, the cell populations or pharmaceutical compositions provided herein are stored in containers designed for storing biological material (e.g., human cells or animal cells) at temperatures as low as 4° C., 0° C., −20° C., or −80° C.

In some embodiments, the cell populations or pharmaceutical compositions provided herein are formulated in freezing media and placed in cryogenic storage units such as liquid nitrogen freezers (−195° C.) or ultra-low temperature freezers (−65° C., −80° C. or −120° C.) for long-term storage of at least about 1 month, 2 months, 3 months, 4 months, 5 months, 6 months, 1 year, 2 years, 3 years, or at least 5 years. The freeze media can contain dimethyl sulfoxide (DMSO), and/or sodium chloride (NaCl), and/or dextrose, and/or dextran sulfate and/or hydroxyethyl starch (HES) with physiological pH buffering agents to maintain pH between about 6.0 to about 6.5, about 6.5 to about 7.0, about 7.0 to about 7.5, about 7.5 to about 8.0 or about 6.5 to about 7.5. The cryopreserved cell populations and pharmaceutical compositions can retain their functionality. In some embodiments, no preservatives are used in the formulation. The cryopreserved cell populations and pharmaceutical compositions can be thawed and administered to (e.g., infused into) multiple patients as allogeneic off-the-shelf cell product. In some embodiments, the cell populations are thawed and further processed by stimulation with antibodies, proteins, peptides, and/or cytokines as described herein before being administered. In some embodiments, the cryopreserved cell populations can be modified to add a targeting moiety as described herein before being administered.

### 5.3 Methods of Uses

The cell populations disclosed herein are enriched in gdT cells with NK-like properties and capable of killing target cells and modulating immune responses. Accordingly, the cell populations and pharmaceutical compositions provided herein can be used as a medicament. In some embodiments, provided herein are methods for treating a disease or disorder in a subject in need thereof comprising administering the cell populations or pharmaceutical compositions described herein to the subject. In some embodiments, provided herein are uses of the cell populations or pharmaceutical compositions described herein for treating a disease or disorder in a subject in need thereof. In some embodiments, provided herein are uses of the cell populations or pharmaceutical compositions described herein for the preparation of a medicament for the treatment of a disease or disorder in a subject in need thereof.

The term “treat” and its grammatical equivalents as used herein in connection with a disease or a condition, or a subject having a disease or a condition refer to an action that suppresses, eliminates, reduces, and/or ameliorates a symptom, the severity of the symptom, and/or the frequency of the symptom associated with the disease or disorder being treated. For example, when used in reference to a cancer or tumor, the term “treat” and its grammatical equivalents refer to an action that reduces the severity of the cancer or tumor, or retards or slows the progression of the cancer or tumor, including (a) inhibiting the growth, or arresting development of the cancer or tumor, (b) causing regression of the cancer or tumor, or (c) delaying, ameliorating or minimizing one or more symptoms associated with the presence of the cancer or tumor.

The term “administer” and its grammatical equivalents as used herein refer to the act of delivering, or causing to be delivered, a therapeutic or a pharmaceutical composition to the body of a subject by a method described herein or otherwise known in the art. The therapeutic can be a compound, a polypeptide, an antibody, a cell, or a population of cells. Administering a therapeutic or a pharmaceutical composition includes prescribing a therapeutic or a pharmaceutical composition to be delivered into the body of a subject.

The terms “effective amount,” “therapeutically effective amount,” and their grammatical equivalents as used herein refer to the administration of an agent to a subject, either alone or as a part of a pharmaceutical composition and either in a single dose or as part of a series of doses, in an amount that is capable of having any detectable, positive effect on any symptom, aspect, or characteristics of a disease, disorder or condition when administered to the subject. The therapeutically effective amount can be ascertained by measuring relevant physiological effects. The exact amount required vary from subject to subject, depending on the age, weight, and general condition of the subject, the severity of the condition being treated, the judgment of the clinician, and the like. An appropriate “effective amount” in any individual case can be determined by one of ordinary skill in the art using routine experimentation.

The term “subject” as used herein refers to any animal (e.g., a vertebrate). The subjects include, but are not limited to, humans, non-human primates, simians, canines, felines, rodents, and the like, which is to be the recipient of a particular treatment. A subject can be a human. A subject can be a mammal. A subject can be a farm animal. As subject can be a pet. A subject can have a particular disease or condition.

In some embodiments, the cell populations and pharmaceutical compositions provided herein can be used in the treatment of cancer, an infectious disease or an inflammatory disease. In some embodiments, the cell populations and pharmaceutical compositions provided herein can be used in modulating an immune response in a subject in need thereof. In some embodiments, provided herein are methods of treating a cancer, an infectious disease or an inflammatory disease in a subject in need thereof, comprising administering a therapeutically effective amount of the cell population described herein. Alternatively, a therapeutically effective amount of the pharmaceutical composition comprising the cell population is administered.

In some embodiments, the disease or disorder can be cancer, tumor, autoimmune disease, neuronal disease, HIV infection, hematopoietic cell-related diseases, metabolic syndrome, pathogenic disease, viral infection, fungal infection, protozoan infection, or bacterial infection. As such, the cell populations provided herein, including those prepared by methods described herein, as well as the pharmaceutical compositions provided herein, can be used in, for example, cancer treatment, autoimmune disease treatment, neuronal disease treatment, human immunodeficiency virus (HIV) eradication, hematopoietic cell-related diseases, metabolic syndrome treatment, pathogenic disease treatment, treatment of viral infection, fungal infection, protozoan infection, and treatment of bacterial infection. In some embodiments, the cell populations and pharmaceutical compositions described herein can be used to treat a disease or disorder associated with abnormal cells. In some embodiments, the disease or disorder is a hyperproliferative disease.

As provided above, in some embodiments, the cell populations or pharmaceutical compositions described herein are modified to have a targeting moiety complexed to the surface of the gdT cells. In some embodiments, the cell populations or pharmaceutical compositions can be used to treat diseases or disorders associated with abnormal cells. In some embodiments, the abnormal cells express an antigen to which the targeting moiety specifically binds, and the interaction between the targeting moiety and the antigen induce an ADCC response of the gdT cells, which results in the killing of the diseases cells.

In some embodiments, provided herein are also the uses of the cell populations or pharmaceutical compositions provided herein in the treatment of a tumor or cancer. In some embodiments, provided herein are methods of treating a tumor or cancer in a subject in need thereof, comprising administering the cell populations or pharmaceutical compositions provided herein to the subject. In some embodiments, the tumor or cancer is a solid tumor. In some embodiments, the tumor or cancer is a hematological cancer, or liquid cancer. In some embodiments, gdT cells of the cell populations or pharmaceutical compositions described herein have a targeting moiety on the cell surface that comprises an antibody that specifically binds to a tumor antigen.

In some embodiments, the disease or disorder that can be treated with the cell populations or pharmaceutical compositions provided herein is acanthoma, acinic cell carcinoma, acoustic neuroma, acral lentiginous melanoma, acrospiroma, acute eosinophilic leukemia, acute lymphoblastic leukemia, acute megakaryoblastic leukemia, acute monocytic leukemia, acute myeloblastic leukemia with maturation, acute myeloid dendritic cell leukemia, acute myeloid leukemia, acute promyelocytic leukemia, adamantinoma, adenocarcinoma, adenoid cystic carcinoma, adenoma, adenomatoid odontogenic tumor, adrenocortical carcinoma, adult t-cell leukemia, aggressive NK-cell leukemia, AIDS-related cancers, AIDS-related lymphoma, alveolar soft part sarcoma, ameloblastic fibroma, anal cancer, anaplastic large cell lymphoma, anaplastic thyroid cancer, angioimmunoblastic t-cell lymphoma, angiomyolipoma, angiosarcoma, appendix cancer, astrocytoma, atypical teratoid rhabdoid tumor, basal cell carcinoma, basal-like carcinoma, b-cell leukemia, b-cell lymphoma, bellini duct carcinoma, biliary tract cancer, bladder cancer, blastoma, bone cancer, bone tumor, brain stem glioma, brain tumor, breast cancer, brenner tumor, bronchial tumor, bronchioloalveolar carcinoma, brown tumor, burkitt's lymphoma, cancer of unknown primary site, carcinoid tumor, carcinoma, carcinoma in situ, carcinoma of the penis, carcinoma of unknown primary site, carcinosarcoma, castleman's disease, central nervous system embryonal tumor, cerebellar astrocytoma, cerebral astrocytoma, cervical cancer, cholangiocarcinoma, chondroma, chondrosarcoma, chordoma, choriocarcinoma, choroid plexus papilloma, chronic lymphocytic leukemia, chronic monocytic leukemia, chronic myelogenous leukemia, chronic myeloproliferative disorder, chronic neutrophilic leukemia, clear-cell tumor, colon cancer, colorectal cancer, craniopharyngioma, cutaneous T-cell lymphoma, Degos disease, dermatofibrosarcoma protuberans, dermoid cyst, desmoplastic small round cell tumor, diffuse large B cell lymphoma, dysembryoplastic neuroepithelial tumor, embryonal carcinoma, endodermal sinus tumor, endometrial cancer, endometrial uterine cancer, endometrioid tumor, enteropathy-associated T-cell lymphoma, ependymoblastoma, ependymoma, epithelioid sarcoma, erythroleukemia, esophageal cancer, esthesioneuroblastoma, Ewing family of tumor, Ewing family sarcoma, Ewing's sarcoma, extracranial germ cell tumor, extragonadal germ cell tumor, extrahepatic bile duct cancer, extramammary Paget's disease, fallopian tube cancer, fetus in fetu, fibroma, fibrosarcoma, follicular lymphoma, follicular thyroid cancer, gallbladder cancer, gallbladder cancer, ganglioglioma, ganglioneuroma, gastric cancer, gastric lymphoma, gastrointestinal cancer, gastrointestinal carcinoid tumor, gastrointestinal stromal tumor, gastrointestinal stromal tumor, germ cell tumor, germinoma, gestational choriocarcinoma, gestational trophoblastic tumor, giant cell tumor of bone, glioblastoma multiforme, glioma, gliomatosis cerebri, glomus tumor, glucagonoma, gonadoblastoma, granulosa cell tumor, hairy cell leukemia, hairy cell leukemia, head and neck cancer, heart cancer, hemangioblastoma, hemangiopericytoma, hemangiosarcoma, hematological malignancy, hepatocellular carcinoma, hepatosplenic T-cell lymphoma, hereditary breast-ovarian cancer syndrome, Hodgkin lymphoma, Hodgkin's lymphoma, hypopharyngeal cancer, hypothalamic glioma, inflammatory breast cancer, intraocular melanoma, islet cell carcinoma, islet cell tumor, juvenile myelomonocytic leukemia, Kaposi sarcoma, Kaposi's sarcoma, kidney cancer, Klatskin tumor, Krukenberg tumor, laryngeal cancer, laryngeal cancer, lentigo maligna melanoma, leukemia, lip and oral cavity cancer, liposarcoma, lung cancer, luteoma, lymphangioma, lymphangiosarcoma, lymphoepithelioma, lymphoid leukemia, lymphoma, macroglobulinemia, malignant fibrous histiocytoma, malignant fibrous histiocytoma, malignant fibrous histiocytoma of bone, malignant glioma, malignant mesothelioma, malignant peripheral nerve sheath tumor, malignant rhabdoid tumor, malignant triton tumor, malt lymphoma, mantle cell lymphoma, mast cell leukemia, mediastinal germ cell tumor, mediastinal tumor, medullary thyroid cancer, medulloblastoma, medulloepithelioma, melanoma, meningioma, merkel cell carcinoma, mesothelioma, metastatic squamous neck cancer with occult primary, metastatic urothelial carcinoma, mixed mullerian tumor, monocytic leukemia, mouth cancer, mucinous tumor, multiple endocrine neoplasia syndrome, multiple myeloma, multiple myeloma, mycosis fungoides, mycosis fungoides, myelodysplastic disease, myelodysplastic syndromes, myeloid leukemia, myeloid sarcoma, myeloproliferative disease, myxoma, nasal cavity cancer, nasopharyngeal cancer, nasopharyngeal carcinoma, neoplasm, neurinoma, neuroblastoma, neuroblastoma, neurofibroma, neuroma, nodular melanoma, non-Hodgkin lymphoma, non-Hodgkin lymphoma, nonmelanoma skin cancer, non-small cell lung cancer, ocular oncology, oligoastrocytoma, oligodendroglioma, oncocytoma, optic nerve sheath meningioma, oral cancer, oral cancer, oropharyngeal cancer, osteosarcoma, ovarian cancer, ovarian cancer, ovarian epithelial cancer, ovarian germ cell tumor, ovarian low malignant potential tumor, Paget's disease of the breast, pancoast tumor, pancreatic cancer, pancreatic cancer, papillary thyroid cancer, papillomatosis, paraganglioma, paranasal sinus cancer, parathyroid cancer, penile cancer, perivascular epithelioid cell tumor, pharyngeal cancer, pheochromocytoma, pineal parenchymal tumor of intermediate differentiation, pineoblastoma, pituicytoma, pituitary adenoma, pituitary tumor, plasma cell neoplasm, pleuropulmonary blastoma, polyembryoma, precursor t-lymphoblastic lymphoma, primary central nervous system lymphoma, primary effusion lymphoma, primary hepatocellular cancer, primary liver cancer, primary peritoneal cancer, primitive neuroectodermal tumor, prostate cancer, pseudomyxoma peritonei, rectal cancer, renal cell carcinoma, respiratory tract carcinoma involving the nut gene on chromosome 15, retinoblastoma, rhabdomyoma, rhabdomyosarcoma, Richter's transformation, sacrococcygeal teratoma, salivary gland cancer, sarcoma, schwannomatosis, sebaceous gland carcinoma, secondary neoplasm, seminoma, serous tumor, Sertoli-Leydig cell tumor, sex cord-stromal tumor, sezary syndrome, signet ring cell carcinoma, skin cancer, small blue round cell tumor, small cell carcinoma, small cell lung cancer, small cell lymphoma, small intestine cancer, soft tissue sarcoma, somatostatinoma, soot wart, spinal cord tumor, spinal tumor, splenic marginal zone lymphoma, squamous cell carcinoma, stomach cancer, superficial spreading melanoma, supratentorial primitive neuroectodermal tumor, surface epithelial-stromal tumor, synovial sarcoma, T-cell acute lymphoblastic leukemia, T-cell large granular lymphocyte leukemia, T-cell leukemia, T-cell lymphoma, T-cell prolymphocytic leukemia, teratoma, terminal lymphatic cancer, testicular cancer, thecoma, throat cancer, thymic carcinoma, thymoma, thyroid cancer, transitional cell cancer of renal pelvis and ureter, transitional cell carcinoma, urachal cancer, urethral cancer, urogenital neoplasm, uterine sarcoma, uveal melanoma, vaginal cancer, Verner-Morrison syndrome, verrucous carcinoma, visual pathway glioma, vulvar cancer, Waldenstrom's macroglobulinemia, Warthin's tumor, Wilms' tumor.

In some embodiments, provided herein are uses of the cell populations or pharmaceutical compositions described herein in an adoptive immunotherapy. The term “adoptive immunotherapy” refers generally to the transfer of immune cells to a subject for the treatment of a disease such as a hyperproliferative disease, a HIV or other viral infectious disease, a fungi infectious disease, a bacteria infectious disease, a protozoan infectious disease, an autoimmune disease, a neuronal disease, a hematopoietic cell-related disease, a metabolic syndrome, or a pathogenic disease.

Adoptive immunotherapies can be autologous, i.e., the cell populations are transferred back into the same patient from which they were obtained, or the immunotherapies can be allogeneic, i.e., the gdT cells from one person can be transferred into a different patient. In instances involving allogeneic transfer, the cell populations are substantially free of ab T cells. For illustrative purposes, a method of treatment can include: obtaining a source cell population (e.g., PBMCs) from a donor individual; culturing the source cell population as described herein to produce a cell population enriched in NK-like gdT cells; and administering the cell population to a recipient individual.

The patient or subject to be treated can be a human patient with a disease or disorder described herein. In some embodiments, the subject is a cancer patient. In some embodiments, the subject is a virus-infected patient (e.g., a CMV-infected or HIV infected patient). In some embodiments, the subject has and/or is being treated for a cancer or tumor.

Because gdT cells are non-MHC restricted, they do not recognize a host into which they are transferred as foreign and are less likely to cause graft-versus-host disease. In some embodiments, the cell populations and pharmaceutical compositions provided herein can be used “off the shelf” and transferred into any recipient for, for example, allogeneic adoptive immunotherapies. As described herein, gdT cells obtained by methods described herein express a cytotoxic profile in the absence of any activation and are therefore likely to be effective at killing tumor cells or other pathogens. For example, the gdT cells obtained as described herein can express one or more, preferably all of CD69, NKG2D, IFN-γ, TNF-α, and Granzyme B in the absence of any activation. In some embodiments, gdT cells obtained by methods described herein express high levels of NKG2D and therefore respond to NKG2D ligands (e.g., MICA) associated with malignancy.

In some instances, a therapeutically effective amount of cell populations or pharmaceutical compositions described above can be administered to a subject (e.g., for treatment of cancer). In some cases, the therapeutically effective amount of cell populations or pharmaceutical compositions include about 10×10¹², about 9×10¹², about 8×10¹², about 7×10¹², about 6×10¹², about 5×10¹², about 4×10¹², about 3×10¹², about 2×10¹², about 1×10¹², about 9×10¹¹, about 8×10¹¹, about 7×10¹¹, about 6×10¹¹, about 5×10¹¹, about 4×10¹¹, about 3×10¹¹, about 2×10¹¹, about 1×10¹¹, about 9×10¹⁰, about 7.5×10¹⁰, about 5×10¹⁰, about 2.5×10¹⁰, about 1×10¹⁰, about 7.5×10⁹, about 5×10⁹, about 2.5×10⁹, about 1×10⁹, about 7.5×10⁸, about 5×10⁸, about 2.5×10⁸, about 1×10⁸, about 7.5×10⁷, about 5×10⁷, about 2.5×10⁷, about 1×10⁷, about 7.5×10⁶, about 5×10⁶, about 2.5×10⁶, about 1×10⁶, about 7.5×10⁵, about 5×10⁵, about 2.5×10⁵, or about 1×10⁵ gdT cells per dose. In some embodiments, a dose can include about 1×10⁷, 2×10⁷, 5×10⁷, 1×10⁸, 2×10⁸, 5×10⁸, 1×10⁹, 2×10⁹, or 5×10⁹ gdT cells. In some embodiments, a dose comprises at least about 1×10⁷, 2×10⁷, 5×10⁷, 1×10⁸, 2×10⁸, 5×10⁸, 1×10⁹, 2×10⁹, or 5×10⁹ cells. In some embodiments, a dose comprises up to about 1×10⁷, 2×10⁷, 5×10⁷, 1×10⁸, 2×10⁸, 5×10⁸, 1×10⁹, 2×10⁹, or 5×10⁹ gdT ells.

In some embodiments, the therapeutically effective amount of cell populations or pharmaceutical compositions can include about 10×10¹², about 9×10¹², about 8×10¹², about 7×10¹², about 6×10¹², about 5×10¹², about 4×10¹², about 3×10¹², about 2×10¹², about 1×10¹², about 9×10¹¹, about 8×10¹¹, about 7×10¹¹, about 6×10¹¹, about 5×10¹¹, about 4×10¹¹, about 3×10¹¹, about 2×10¹¹, about 1×10¹¹, about 9×10¹⁰, about 7.5×10¹⁰, about 5×10¹⁰, about 2.5×10¹⁰, about 1×10¹⁰, about 7.5×10⁹, about 5×10⁹, about 2.5×10⁹, about 1×10⁹, about 7.5×10⁸, about 5×10⁸, about 2.5×10⁸, about 1×10⁸, about 7.5×10⁷, about 5×10⁷, about 2.5×10⁷, about 1×10⁷, about 7.5×10⁶, about 5×10⁶, about 2.5×10⁶, about 1×10⁶, about 7.5×10⁵, about 5×10⁵, about 2.5×10⁵, or about 1×10⁵ gdT cells over the course of treatment. In some embodiments, the therapeutically effective amount of cell populations or pharmaceutical compositions can include at least 10×10¹², at least 9×10¹², at least 8×10¹², at least 7×10¹², at least 6×10¹², at least 5×10¹², at least 4×10¹², at least 3×10¹², at least 2×10¹², at least 1×10¹², at least 9×10¹¹, at least 8×10¹¹, at least 7×10¹¹, at least 6×10¹¹, at least 5×10¹¹, at least 4×10¹¹, at least 3×10¹¹, at least 2×10¹¹, at least 1×10¹¹, at least 9×10¹⁰, at least 7.5×10¹⁰, at least 5×10¹⁰, at least 2.5×10¹⁰, at least 1×10¹⁰, at least 7.5×10⁹, at least 5×10⁹, at least 2.5×10⁹, at least 1×10⁹, at least 7.5×10⁸, at least 5×10⁸, at least 2.5×10⁸, at least 1×10⁸, at least 7.5×10⁷, at least 5×10⁷, at least 2.5×10⁷, at least 1×10⁷, at least 7.5×10⁶, at least 5×10⁶, at least 2.5×10⁶, at least 1×10⁶, at least 7.5×10⁵ at least 5×10⁵, at least 2.5×10⁵, or at least 1×10⁵ gdT cells over the course of treatment. In some embodiments, the therapeutically effective amount of cell populations or pharmaceutical compositions can include up to 10×10¹², up to 9×10¹², up to 8×10¹², up to 7×10¹², up to 6×10¹², up to 5×10¹², up to 4×10¹², up to 3×10¹², up to 2×10¹², up to 1×10¹², up to 9×10¹¹, up to 8×10¹¹, up to 7×10¹¹, up to 6×10¹¹, up to 5×10¹¹, up to 4×10¹¹, up to 3×10¹¹, up to 2×10¹¹, up to 1×10¹¹, up to 9×10¹⁰, up to 7.5×10¹⁰, up to 5×10¹⁰, up to 2.5×10¹⁰, up to 1×10¹⁰, up to 7.5×10⁹, up to 5×10⁹, up to 2.5×10⁹, up to 1×10⁹, up to 7.5×10⁸, up to 5×10⁸, up to 2.5×10⁸, up to 1×10⁸, up to 7.5×10⁷, up to 5×10⁷, up to 2.5×10⁷, up to 1×10⁷, up to 7.5×10⁶, up to 5×10⁶, up to 2.5×10⁶, up to 1×10⁶, up to 7.5×10⁵, up to 5×10⁵, up to 2.5×10⁵, or up to 1×10⁵ gdT cells over the course of treatment.

In some embodiments, a dose of the therapeutically effective amount of cell populations or pharmaceutical compositions can include about 1×10⁶, 1.1×10⁶, 2×10⁶, 3.6×10⁶, 5×10⁶, 1×10⁷, 1.8×10⁷, 2×10⁷, 5×10⁷, 1×10⁸, 2×10⁸, or 5×10⁸ gdT cells/kg. In some embodiments, a dose can include up to about 1×10⁶, 1.1×10⁶, 2×10⁶, 3.6×10⁶, 5×10⁶, 1×10⁷, 1.8×10⁷, 2×10⁷, 5×10⁷, 1×10⁸, 2×10⁸, or 5×10⁸ gdT cells/kg. In some embodiments, a dose can include about 1.1×10⁶-1.8×10⁷ gdT cells/kg.

In some embodiments, the subject is administered one dose during the treatment. In some embodiments, the subject is administered at least two doses during the treatment. In some embodiments, the subject receives an initial dose and one or more (e.g., 2, 3, 4, or 5) subsequent administrations. In one embodiment, the one or more subsequent administrations are administered less than 15 days (e.g., 14, 13, 12, 11, 10, 9, 8, 7, 6, 5, 4, 3, or 2 days) after the previous administration after the previous administration. For illustrative purposes, in some embodiments, the subject receives a total of about 5×10⁷ gdT cells over the course of three administrations, e.g., the subject receives an initial dose of 3×10⁷ gdT cells, a second administration of 1.5×10⁷ gdT cells, and a third administration of 1.5×10⁷ gdT cells, wherein each administration is administered less than 4, 3, or 2 days after the previous administration. A person of ordinary skill in the art would be able to adjust and optimize the doses as necessary and appropriate.

In some embodiments, one or more additional therapeutic agents can be administered to the subject. In some embodiments, the cell populations and pharmaceutical compositions described herein are used as medicament for the treatment of diseases as an adjunct to, or in conjunction with, other established therapies normally used in the treatment of such diseases. The additional therapeutic agent can be administered prior to, concurrently with, or after the administration of the cell populations or pharmaceutical populations provided herein. The additional therapeutic agent can be selected from the group consisting of an immunotherapeutic agent, a cytotoxic agent, a growth inhibitory agent, a radiation therapy agent, an anti-angiogenic agent, or any combination thereof. The additional therapeutic agent can be an immunotherapeutic agent, which can act on a target within the subject's body (e.g., the subject's own immune system) and/or on the transferred gdT cells. In some embodiments, the additional therapeutic agent is an antibody targeting a tumor antigen.

The administration of the compositions can be carried out in any convenient manner. The cell populations and pharmaceutical compositions described herein can be administered to a subject transarterially, subcutaneously, intradermally, intratumorally, intranodally, intramedullary, intramuscularly, by intravenous injection, or intraperitoneally, e.g., by intradermal or subcutaneous injection. The compositions of gdT cells can be injected directly into a tumor, lymph node, or site of infection.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### 5.4 Examples

The examples provided below are for purposes of illustration only, which are not intended to be limiting unless otherwise specified. Thus, the invention should in no way be construed as being limited to the following examples, but rather, should be construed to encompass any and all variations which become evident as a result of the teaching provided herein.

Exemplary genes and polypeptides are described herein with reference to GenBank numbers, GI numbers and/or SEQ ID NOs. It is understood that one skilled in the art can readily identify homologous sequences by reference to sequence sources, including but not limited to GenBank (ncbi.nlm.nih.gov/genbank/) and EMBL (embl.org/).

Some culture media used in the studies were referred to as “complete growth medium” or “complete medium.” Complete growth media were RPMI-1640 based and supplemented with 1-30 vol % HPL and 100-2500 IU/mL (0.0612-1.53 μg/mL) human IL-2, and complete media were RPMI-1640 based and supplemented with 1-30 vol % HPL. The actual concentrations of HPL and IL-2 used in the studies described below are separated indicated.

5.4.1 Preparation of Compositions Enriched in gdT Cells with NK-Like Properties

A cell population enriched in gdT cells (T cells with NK-like properties) was prepared following the procedures illustrated in FIG. 1B: On Day 0, a vial of cryopreserved human PBMCs were thawed in a 37° C. water bath. 1 mL of the thawed PBMCs were resuspended and centrifuged at 400×g at room temperature for 3-5 minutes. The cells were resuspended, and 3×10⁷ of the resuspended cells were transferred into a G-Rex device containing complete growth medium (5% (v/v) HPL and 700 IU/mL (0.4284 μg/mL) human IL-2) further supplemented with 1 μM zoledronate, and the culture volume was filled up to the max capacity of the G-Rex device. Human IL-2 was replenished between Day 2 and Day 4. On Day 6, TCRα/β T cells in the expanded cell population were labeled with anti-TCRα/β-Biotin and depleted using anti-Biotin MicroBeads according to manufacturer's instruction. The eluted cells were reseeded in a larger G-Rex device at cell density 1×10⁶ cells/mL. Between Day 8 and Day 16, cells were reseeded, medium changed, and/or human IL-2 replenished as needed. The cultured cells were harvested on Day 16 for subsequent application.

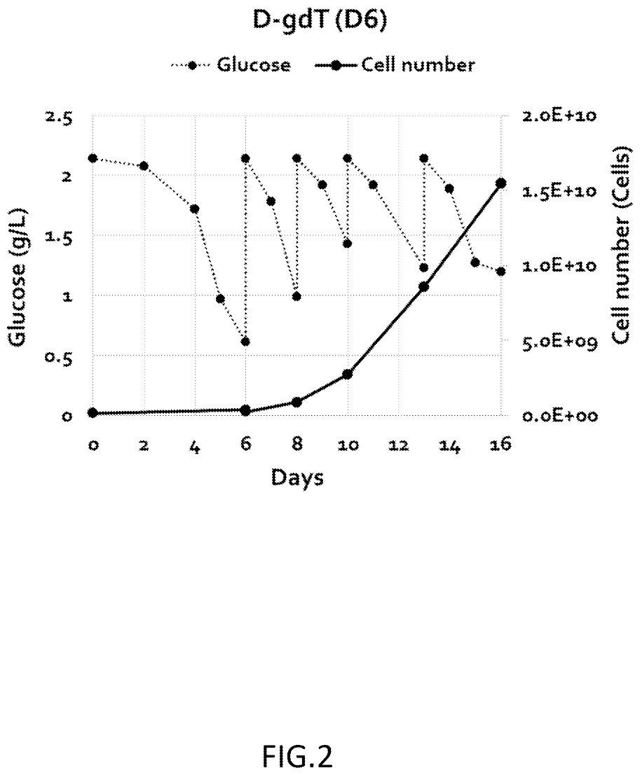

Glucose level was monitored on Days 0, 2, 4, 5, 6, 7, 8, 9, 10, 11, 13, 14, 15 and 16 (Kabelitz et al., 2020. Cell Mol Immunol. 17(9):925-939). Cell numbers were monitored on Days 0, 6, 8, 10, 13, and 16. Each sample of the cell suspensions obtained on different days was mixed with an equal volume of Trypan blue, and the total cell number in the culture was calculated. As shown in FIG. 2, methods described above rapidly expanded the 3×10⁷ human PBMCs to a population of 1.55×10¹⁰ cells in 16 days.

### 5.4.2 Characterization of the Resulting Cell Population by Flow Cytometry

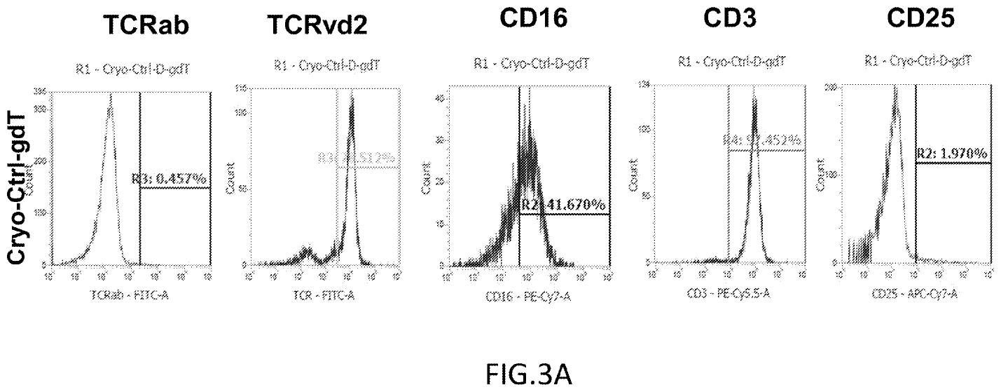

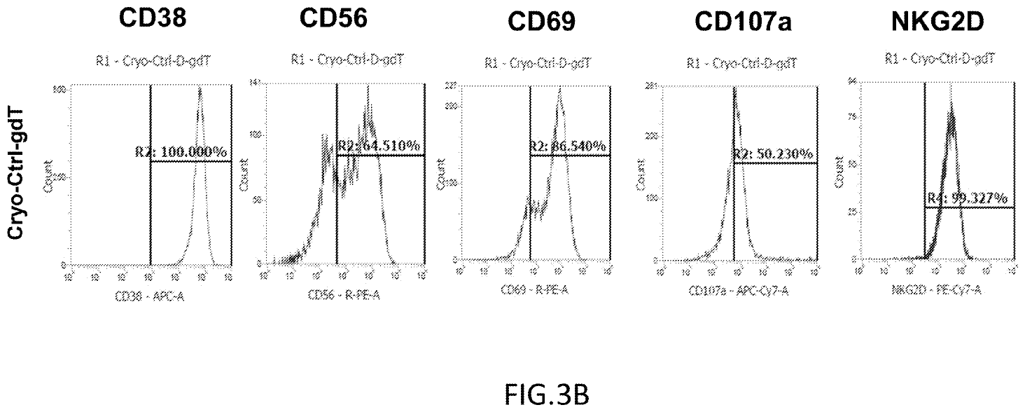

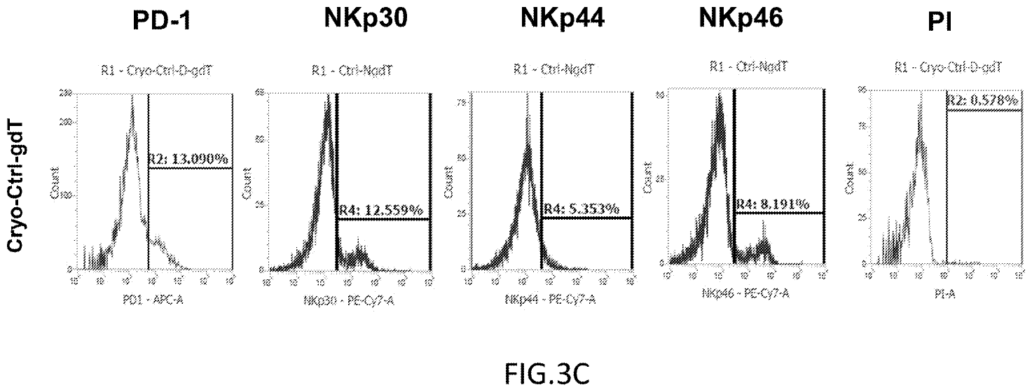

Cells obtained on Day 16 in the study described in Section 5.4.1 above (hereinafter referred to as the “Day 16 resulting cell population” or “Ctrl-gdT cells”) were characterized by flow cytometry. The cells were first centrifuged at room temperature at 400×g for 3 minutes. Supernatant was discarded and cell pellet was resuspended and washed with 1 mL of Dulbecco's Phosphate-Buffered Saline (DPBS). Cell suspension was centrifuged again, and the supernatant was removed. Cell pellet was resuspended with DPBS, and 0.1 mL of cell suspension (5×10⁵ cells) was aliquoted to 1.5 mL Eppendorf tubes. 5×10⁵ cells were then stained with fluorescent dye-conjugated antibodies against TCRα/β, TCRVδ2, CD16, CD3, CD25, CD38, CD56, CD69, CD107a, NKG2D, PD-1, NKp30, NKp44, and NKp46, respectively (all antibodies were purchased from BioLegend). Viability of the cells in the composition was determined by propidium iodide (PI, ThermoFisher Scientific) negative staining. Cells were centrifuged at room temperature at 400×g for 3 minutes. Supernatant was removed, and cell pellet was resuspended with 1 mL of DPBS. Centrifugation was repeated, and 0.5 mL of DPBS-resuspended cells were analyzed for percentage and/or mean fluorescence intensity (MFI) of TCRα/β+, TCRVδ2+, CD16+, CD3+, CD25+, CD38+, CD56+, CD69+, CD107a+, NKG2D+, PD-1+, NKp30+, NKp44+, NKp46+ and PI+ populations by flow cytometry. Results are shown in FIGS. 3A-3C and summarized in the table below.

| | PI+ | TCRα/β+ | TCRVδ2+ | CD16+ | CD3+ | CD25+ | CD38+ | CD56+ |
|---|---|---|---|---|---|---|---|---|
| % | 0.58 | 0.46 | 78.51 | 41.67 | 97.45 | 1.97 | 100 | 64.51 |
| | CD69+ | CD107a+ | NKG2D+ | PD-1+ | NKp30+ | NKp44+ | NKp46+ | |
| % | 86.54 | 50.23 | 99.33 | 13.09 | 12.56 | 5.35 | 8.19 | |

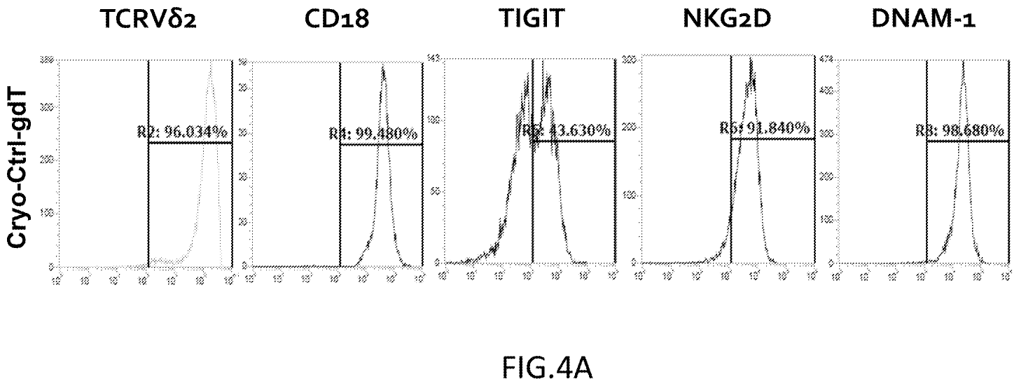

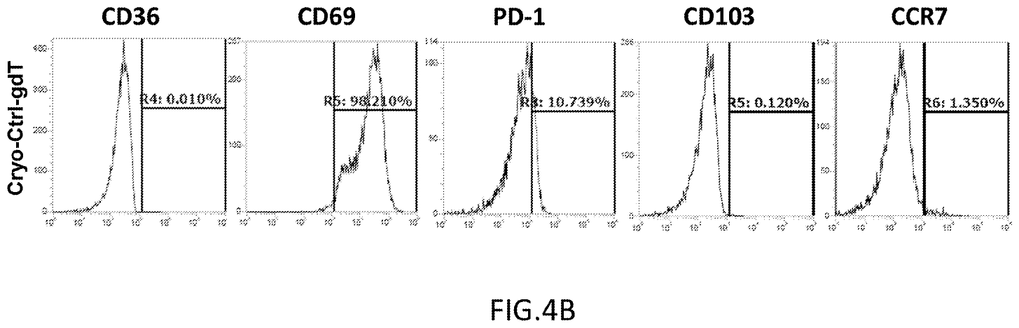

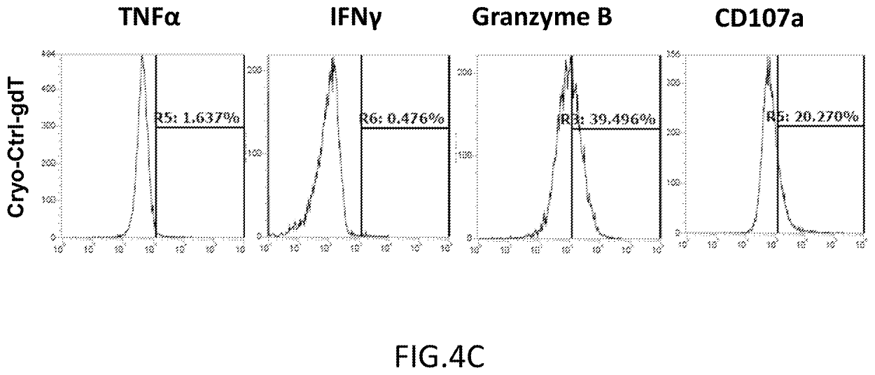

The TCRVδ2+cells in the Day 16 resulting cell population were further analyzed for percentages and/or MFI of TCRVδ2+, CD18+, TIGIT+, NKG2D+, DNAM-1+, CD36+, CD69+, PD-1+, CD103+, CCR7+, TNFα+, IFNγ+, granzyme B+, and CD107a+ populations by flow cytometry. Specifically, cells in the Day 16 resulting cell population were co-stained with fluorescent dye-conjugated antibody against TCRVδ2 along with CD18, TIGIT, NKG2D, DNAM-1, CD36, CD69, PD-1, CD103, CCR7, TNFα, IFNγ, granzyme B, CD107a, CD45RA and CD27 (all antibodies were purchased from BioLegend). Viability was determined by PI staining. The PI−TCRVβ2+-gated populations (PI negative and TCRVδ2 positive cells) were further characterized in percentage/MFI of the above-mentioned markers. The percentages were calculated using the number of PI−TCRVβ2+cells (i.e., the gdT cells) as the total number (denominator), and the MFI values were determined in respective gated marker-positive gdT populations. Results are shown in FIGS. 4A-4C and summarized below.

| | CD18+ | TIGIT+ | NKG2D+ | DNAM1+ | CD36+ | CD69+ | PD-1+ |
|---|---|---|---|---|---|---|---|
| % | 99.48 | 43.63 | 91.84 | 98.68 | 0.01 | 98.21 | 10.74 |
| MFI | 48377 | 5330 | 6193 | 25956 | NA | 31100 | 1729 |
| | CD103+ | CCR7+ | TNFα+ | IFNγ+ | GRZB+ | CD107a+ | |
| % | 0.12 | 1.35 | 1.64 | 0.48 | 39.50 | 20.27 | |
| MFI | 1991 | 4307 | 2798 | 3105 | 2818 | 3487 | |

Conversion of MFI to average Number of Molecules per Cell (“NMC”): The MFI values in the flow cytometry results were also converted to NMC. As used herein, NMC refers to the number of molecules detected on the surface of cells on average. For example, if 50% of a cell population detectably express receptor X, with each cell expressing about 400 molecules of receptor X, the NMC of receptor X in this receptor X-expressing sub-population should be 400 (number of cells with detectable expression used as the denominator), not 200 (number of all cells used as the denominator). To convert MFI to NMC, standard curves derived from Quantum™ Simply Cellular® kit (Bangs Laboratories, Inc. #817) were developed. Five bottles of microspheres (4 populations “#1, #2, #3 and #4” coated with increasing amounts of anti-mouse IgG Fc antibody, 1 uncoated blank) in the Quantum™ Simply Cellular® kit were used. 10 μL of anti-mouse IgG Fc antibody-bound microspheres, including #1, #2, #3 and #4, and blank microsphere were individually incubated with 5 μg/mL of one of the corresponding antibodies in total 0.1 mL reaction volume at room temperature for 10 minutes.

Microspheres were washed with 0.5 mL of DPBS and the cell suspension was centrifuged at 400×g at room temperature for 5 minutes. The supernatant was removed and the suspended QSC (Quantum™ Simply Cellular® kit) microspheres were analyzed by flow cytometry. Acquired MFI of each microsphere was inserted into respective columns of manufacturer-provided calculation sheet (QuickCal V2.3) to generate the corresponding standard curve for each antibody following manufacturer's instruction. For each antibody, after developing the standard curve, the MFI of the corresponding antibody-stained cell population was next inserted to the QuickCal sheet to convert into number of the corresponding receptors on cell surface on average.

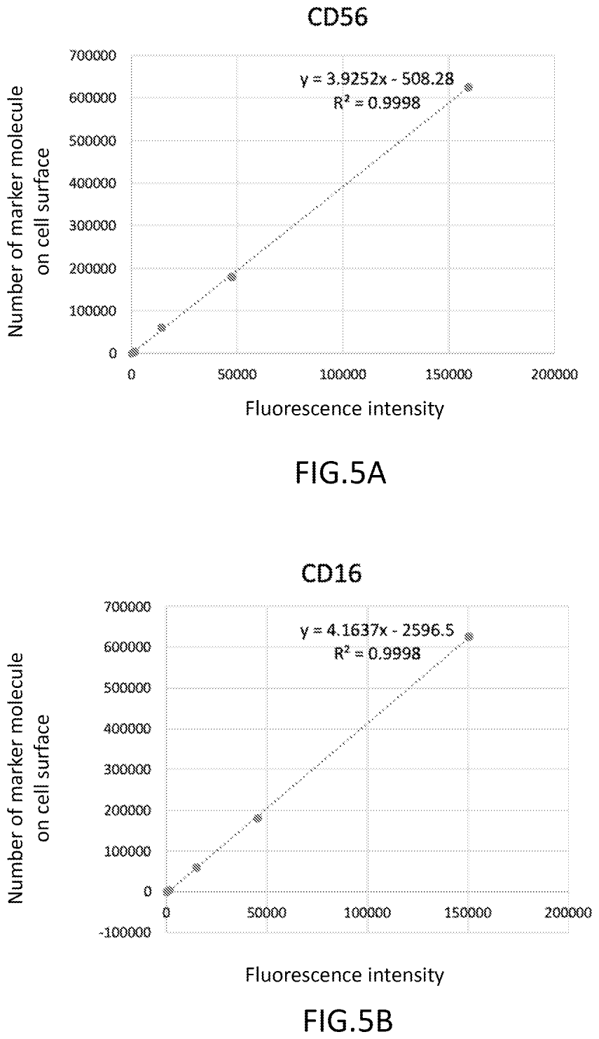

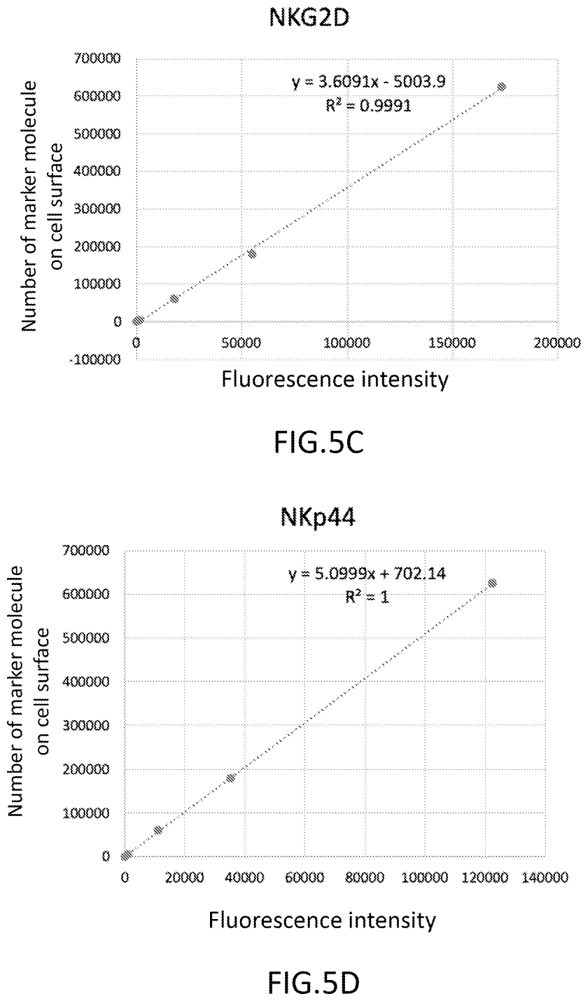

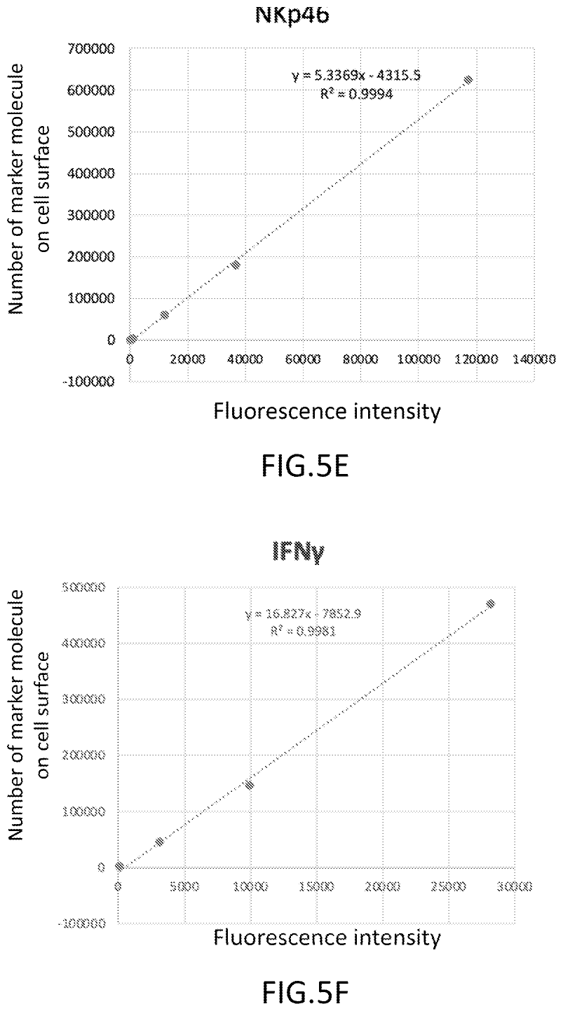

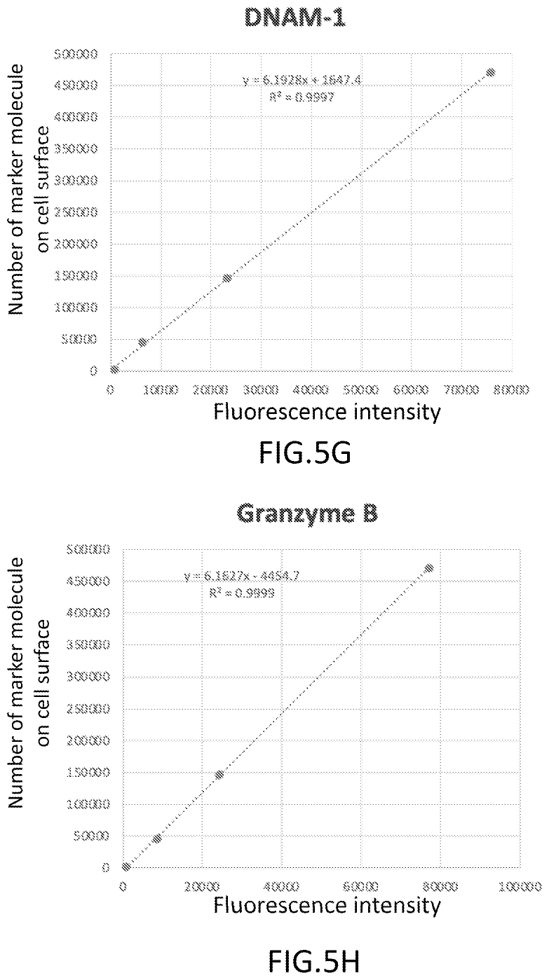

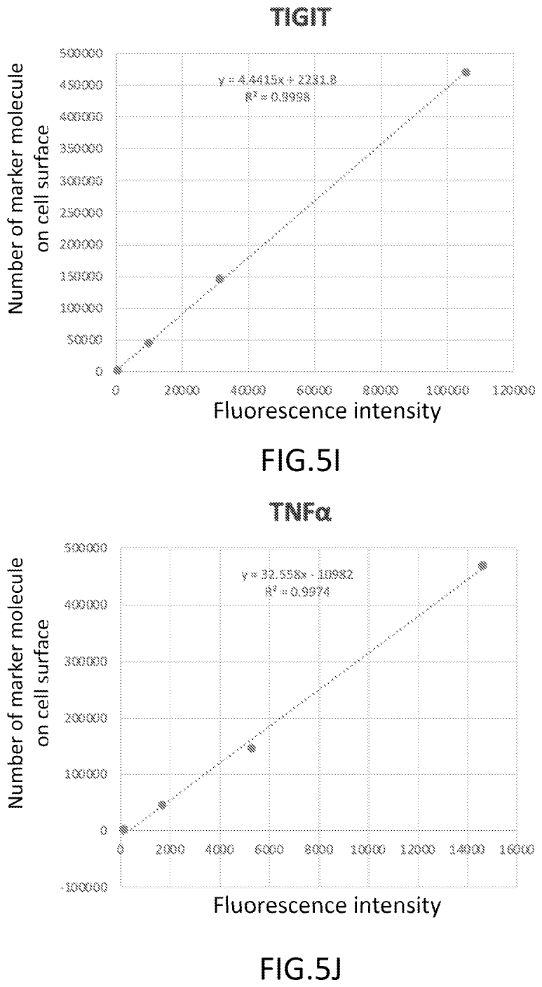

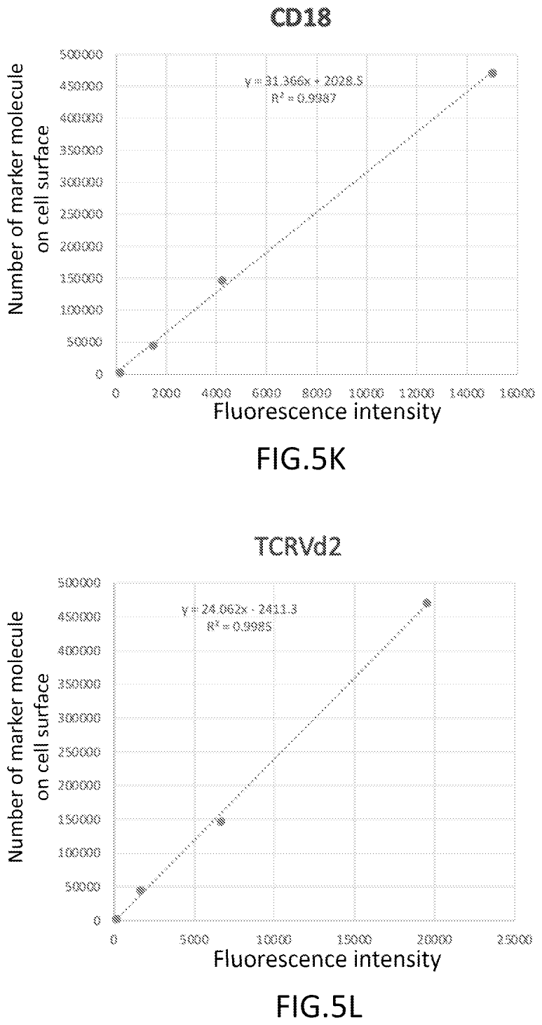

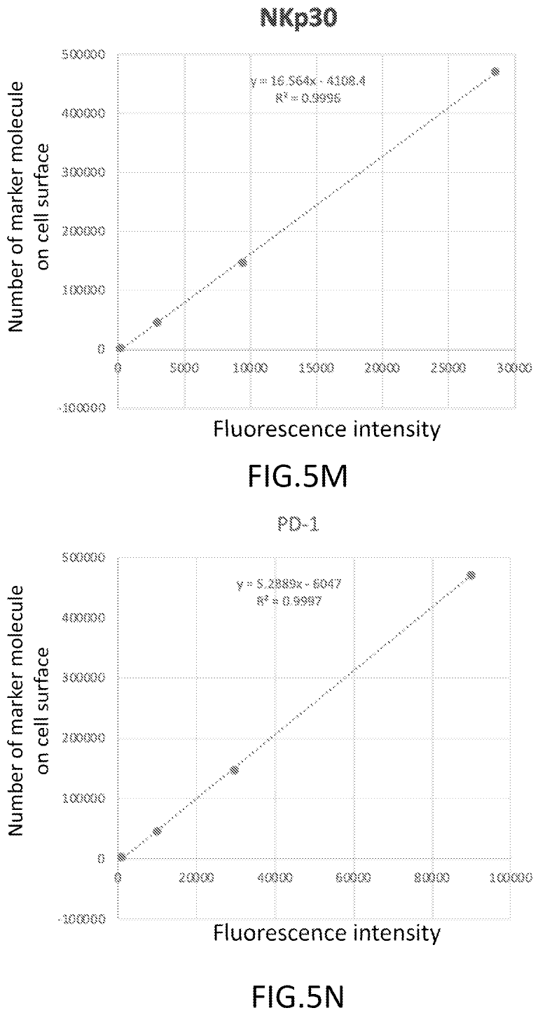

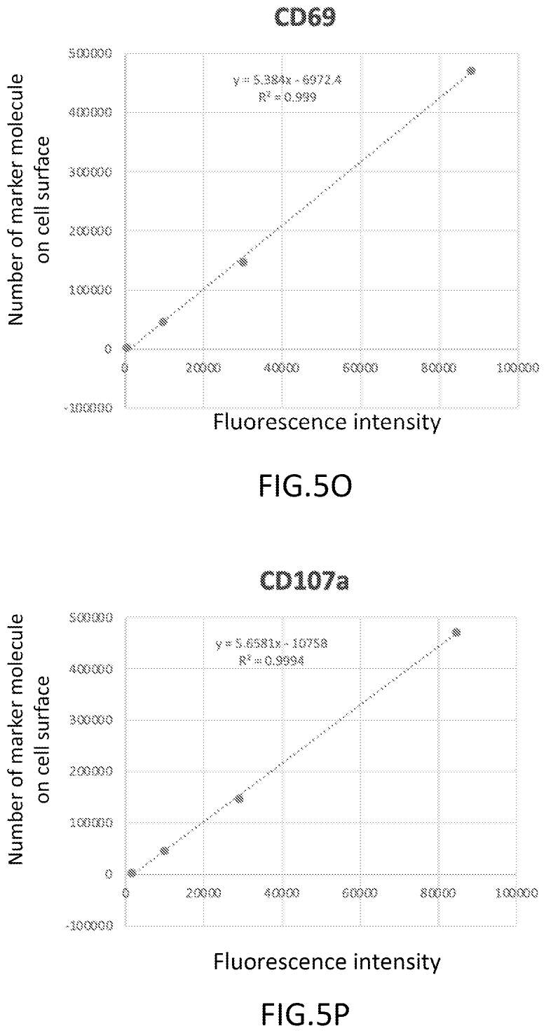

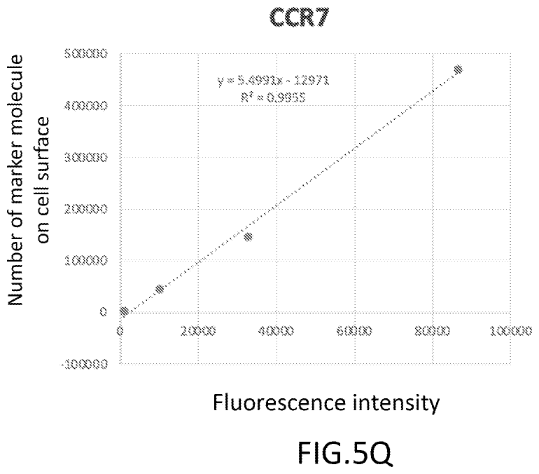

The following MFI/NMC conversions are provided below for exemplary purpose. For example, in studies described in Section 5.4.2, the MFI of the “NKG2D⁺PI⁻TCRVδ2⁺ cell population” (or the NKG2D-expressing gdT population) was 6193; the QuickCal sheet converted this MFI value into an NMC of 17347 based on the standard curve for the mouse anti-human NKG2D IgG used in the study (FIG. 5C; x stands for the MFI and y stands for the NMC), meaning that 17347 NKG2D molecules were detected on the cell surface on average in this study. Notably, because the “NKG2D⁺PI⁻TCRVδ2⁺ cell population” was gated for measuring the MFI value, the calculated NMC value corresponded to the average number of NKG2D molecules per cell in the NKG2D-expressing subpopulation of the entire gdT population. For another example, in studies described in Section 5.4.8, the MFI of the “PI⁻TCRVδ2⁺ cell population” (or the gdT cell population) in 5 vol % HPL group was 18488; QuickCal sheet converted this MFI value into an NMC of 61721 based on the standard curve for the mouse anti-human NKG2D IgG used in the study (FIG. 5C), meaning that 61721 NKG2D molecules were detected on the cell surface on average in this study. Here, as the MFI value was measured in the gated “PI⁻TCRVδ2+cell population,” the calculated NMC value corresponded to the NMC of NKG2D in the entire gdT cell population.

The standing curves for the following antibodies are shown in FIGS. 5A-5Q: PE-conjugated mouse anti-human CD56 IgG (FIG. 5A), PE-Cy7-conjugated mouse anti-human CD16 IgG (FIG. 5B), mouse anti-human NKG2D IgG (FIG. 5C), mouse anti-human NKp44 IgG (FIG. 5D), mouse anti-human NKp46 IgG (FIG. 5E), mouse anti-human IFNγ IgG (FIG. 5F), mouse anti-human DNAM-1 IgG (FIG. 5G), Alexa647-conjugated mouse anti-human granzyme B IgG (FIG. 5H), mouse anti-human TIGIT IgG (FIG. 5I), FITC-conjugated mouse anti-human TNFα IgG (FIG. 5J), mouse anti-human CD18 IgG (FIG. 5K), mouse anti-human TCRVd2 IgG (FIG. 5L), mouse anti-human NKp30 IgG (FIG. 5M), mouse anti-human PD-1 IgG (FIG. 5N); PE-conjugated mouse anti-human CD69 IgG (FIG. 5O); APC-conjugated mouse anti-human CD107a IgG (FIG. 5P); and mouse anti-human CCR7 IgG (FIG. 5Q).

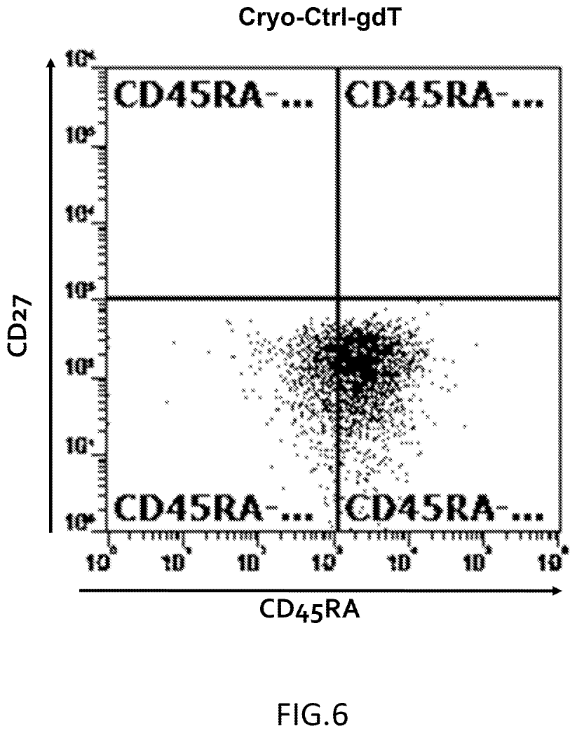

The phenotype of PI−TCRVβ2+-gated populations were also analyzed to distinguish between naïve (CD45RA+CD27+), CM (CD45RA−CD27+), EM (CD45RA−CD27−) and TDEM (CD45RA+CD27−) cells. As shown in FIG. 6, the PI−TCRVβ2+-gated cells were primarily enriched in EM cells (CD45RA−CD27−; 26.43%) and TDEM cells (CD45RA+CD27−; 73.57%).

Expression of NK cytotoxicity receptors (CD56, CD16, DNAM-1, NKG2D, NKp44 and NKp46) and degranulation markers (CD107a) potentiates gdT cells with NK-like anti-tumor activity, whereas enrichment of EM and TDEM cells in gdT cells helps their localization at inflammatory microenvironment of tumor. CD69 expression represents activation of gdT cells. As such, the culturing methods described herein rapidly and selectively expanded the gdT cells with NK-like properties in PBMCs, as demonstrated with the increased expression of (1) NK cytotoxicity receptors (CD56, CD16, DNAM-1, NKG2D, NKp44 and NKp46), (2) degranulation markers (CD107a), and (3) activation marker (CD69) in cells of the Day 16 resulting cell population.

### 5.4.3 Preparation of ACE-gdT Cells

Preparation of ACE-gdT-CD20 cells: ACE-gdT-CD20 cells were obtained by binding rituximab (commercially available anti-CD20 antibody) to Ctrl-gdT cells (the Day 16 resulting cell population prepared as described in section 5.4.1) using a cell linker and a rituximab linker which were complementary, which included the following steps:
(A′) preparing the cell linker and binding the cell linker (a first ssDNA) to the Ctrl-gdT cell to prepare an gdT-ssDNA conjugate;
(B′) preparing rituximab linker and binding the rituximab linker (a second ssDNA complementary to the first ssDNA) to rituximab to prepare the rituximab-ssDNA conjugate; and
(C′) mixing gdT-ssDNA conjugate and 100-500p of rituximab-ssDNA conjugate to prepare ACE-gdT-CD20 cells by allowing the complementary ssDNA linkers to hybridize.

The step (A′) included the following steps (a1′)˜(a4′):
(a1′) a first ssDNA was obtained (SEQ ID NO:1, SEQ ID NO:2, or SEQ ID NO:3);
(a2′) the 5′ end of the first ssDNA was modified with a thiol group (5′ end thiol-modified first ssDNA) to obtain the cell linker stock (see e.g., Zimmermann, J, 2010; also commercially available from Integrated DNA Technologies);
(a3′) 10-500 μL cell linker stock and 0.1-10 μL NHS-Maleimide (SMCC, commercially available from Fisher Scientific) were mixed and incubated for 1-60 minute(s); and
(a4′) the resulting mixtures from Step (a3′) were mixed with 1×10⁶-1×10⁹ Ctrl-gdT cells and incubated for 1-60 minutes.

The step (B′) included the following steps (b1′)˜(b4′):
(b1′) a second ssDNA was obtained (SEQ ID NO:4, SEQ ID NO:5, or SEQ ID NO: 6);
(b2′) the 5′ end of the second ssDNA was modified with a thiol group (5′ end thiol-modified second ssDNA) to obtain the rituximab linker stock (see e.g., Zimmermann, J, 2010; also commercially available from Integrated DNA Technologies);
(b3′) 10-500 μL rituximab linker stock and 0.1-10 μL NHS-Maleimide (SMCC, commercially available from Fisher Scientific) were mixed and incubated for 1-60 minute(s); and
(b4′) the resulting mixtures from Step (b3′) were mixed with 10-100 μL rituximab stock (Rituxan®) and incubated for 10 minutes to 3 hours.

Preparation of ACE-gdT-HER2 cells: ACE-gdT-HER2 cells were obtained by binding trastuzumab (commercially available anti-HER2 antibody) to Ctrl-gdT cells (the Day 16 resulting cell population prepared as described in section 5.4.1) using a cell linker and a trastuzumab linker which were complementary, which included the following steps:
(A″) preparing the cell linker and binding the cell linker (a first ssDNA) to the Ctrl-gdT cell to prepare an gdT-ssDNA conjugate;
(B″) preparing trastuzumab linker and binding the trastuzumab linker (a second ssDNA complementary to the first ssDNA) to trastuzumab to prepare the trastuzumab-ssDNA conjugate; and
(C″) mixing gdT-ssDNA conjugate and 100-500 μL of trastuzumab-ssDNA conjugate to prepare ACE-gdT-HER2 cells by allowing the complementary ssDNA linkers to hybridize.

The step (A″) included the following steps (a1″)˜(a4″):
(a1″) a first ssDNA was obtained (SEQ ID NO: 1, SEQ ID NO:2, or SEQ ID NO:3);
(a2″) the 5′ end of the first ssDNA was modified with a thiol group (5′ end thiol-modified first ssDNA) to obtain the cell linker stock (see e.g., Zimmermann, J, 2010; also commercially available from Integrated DNA Technologies);
(a3″) 10-500 μL cell linker stock and 0.1-10 μL NHS-Maleimide (SMCC, commercially available from Fisher Scientific) were mixed and incubated for 1-60 minute(s); and
(a4″) the resulting mixtures from Step (a3″) were mixed with 1×10⁶-1×10⁹ Ctrl-gdT cells and incubated for 1-60 minutes.

The step (B″) included the following steps (b1″)˜(b4″):
(b1″) a second ssDNA was obtained (SEQ ID NO:4, SEQ ID NO:5, or SEQ ID NO:6);
(b2″) the 5′ end of the second ssDNA was modified with a thiol group (5′ end thiol-modified second ssDNA) to obtain the trastuzumab linker stock (see e.g., Zimmermann, J, 2010; also commercially available from Integrated DNA Technologies);
(b3″) 10-500 μL trastuzumab linker stock and 0.1-10 μL NHS-Maleimide (SMCC, commercially available from Fisher Scientific) were mixed and incubated for 1-60 minute(s); and
(b4′) the resulting mixtures from Step (b3″) were mixed with 10-100 μL trastuzumab stock and incubated for 10 minutes to 3 hours.

### 5.4.4 Characterization of ACE-gdT Cells by Flow Cytometry

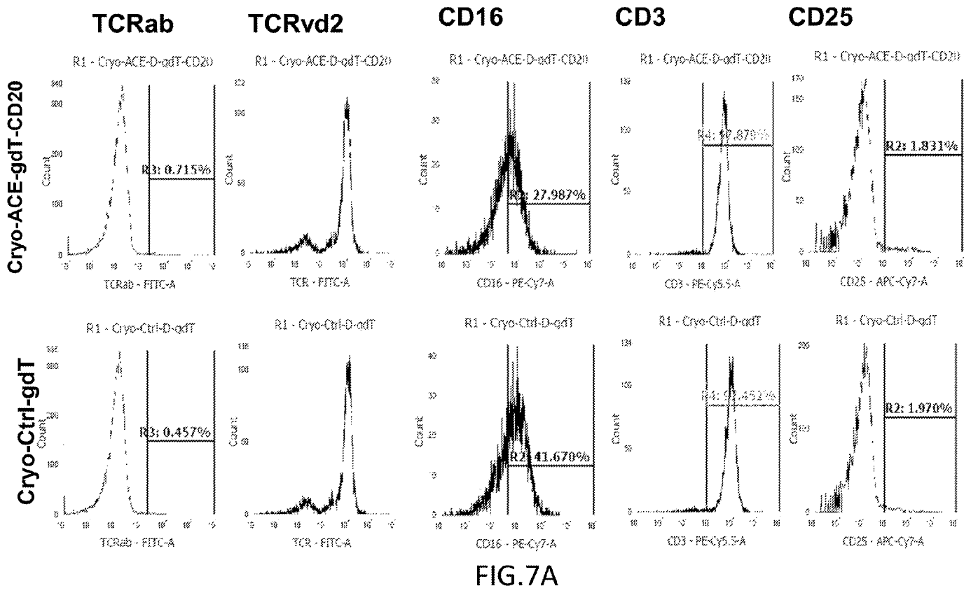

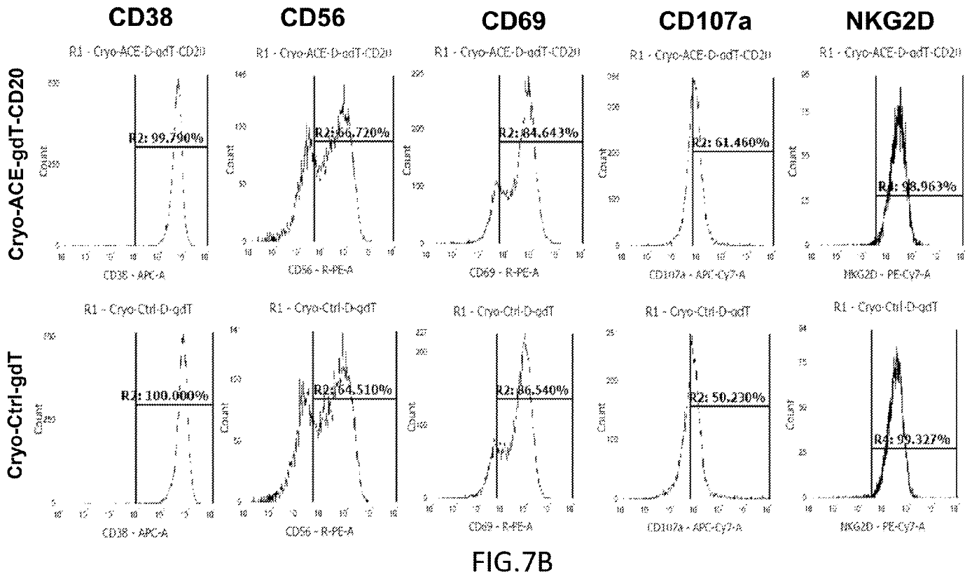

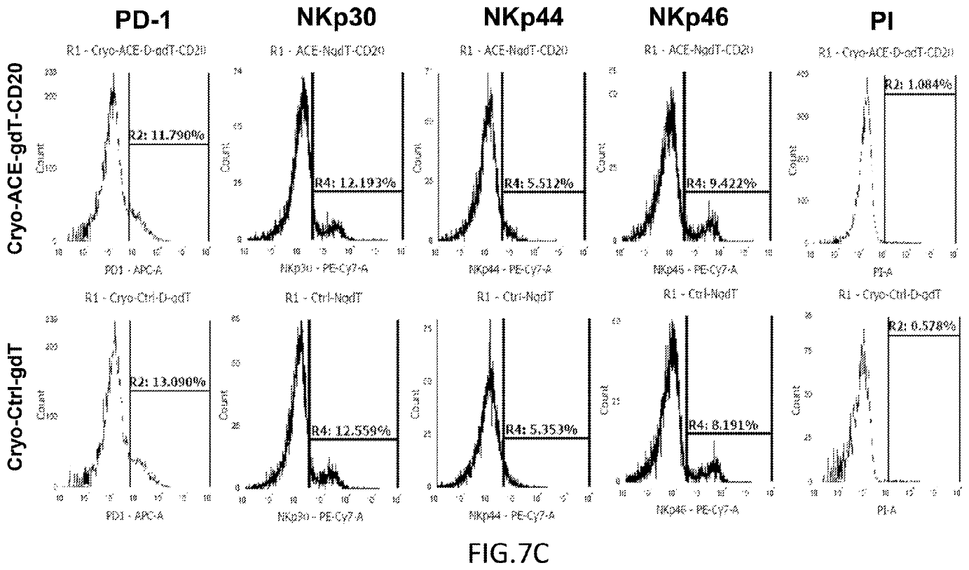

The Day 16 resulting cell population described in Section 5.4.1 above was divided into 2 groups: one used as the control group (Ctrl-gdT) and the other further modified to prepare the ACE-gdT-CD20 group using methods described in Section 5.4.3 above. Briefly, half million of cells of each group were individually stained with fluorescent dye-conjugated antibody against TCRα/β, TCRVδ2, CD16, CD3, CD25, CD38, CD56, CD69, CD107a, NKG2D, PD-1, NKp30, NKp44, and NKp46, respectively. Viability of the cells in the composition was determined by PI negative staining. Cells were centrifuged at room temperature at 400×g for 3 minutes. Supernatant was removed, and cell pellet was resuspended with 1 mL of DPBS. Centrifugation was repeated, and 0.5 mL of DPBS-resuspended cells were analyzed for percentages of TCRα/β+, TCRVδ2+, CD16+, CD3+, CD25+, CD38+, CD56+, CD69+, CD107a+, NKG2D+, PD-1+, NKp30+, NKp44+, NKp46+ and PI+populations by flow cytometry. Results are shown in FIGS. 7A-7C and summarized below.

| % | Cryo-Ctrl-gdT | Cryo-ACE-gdT-CD20 |
|---|---|---|
| PI+ | 0.58 | 1.08 |
| TCRα/β+ | 0.46 | 0.72 |
| TCRVδ2+ | 78.51 | 79.16 |
| CD16+ | 41.67 | 27.99 |
| CD3+ | 97.45 | 97.88 |
| CD25+ | 1.97 | 1.83 |
| CD38+ | 100 | 99.79 |
| CD56+ | 64.51 | 66.72 |
| CD69+ | 86.54 | 84.64 |
| CD107a+ | 50.23 | 61.46 |
| NKG2D+ | 99.33 | 98.96 |
| PD-1+ | 13.09 | 11.79 |
| NKp30+ | 12.56 | 12.19 |
| NKp44+ | 5.35 | 5.51 |
| NKp46+ | 8.19 | 9.42 |

** The prefix “Cryo-” means that the cell populations had been cryopreserved and thawed before the studies.

TCRVδ2+gdT cells: The TCRVδ2+cells in each group were further analyzed for percentages and/or MFI of TCRVδ2+, CD18+, TIGIT+, NKG2D+, DNAM-1+, CD36+, CD69+, PD-1+, CD103+, CCR7+, TNFα+, IFNγ+, granzyme B+, CD107a+, CD45RA, and CD27 populations by flow cytometry. Cells in control group or cells in Cryo-ACE-gdT-CD20 group were co-stained with fluorescent dye-conjugated antibody against TCRVδ2 CD18, TIGIT, NKG2D, DNAM-1, CD36, CD69, PD-1, CD103, CCR7, TNFα, IFNγ, granzyme B (GZMB), CD107a, CD45RA and CD27. Viability was determined by PI staining.

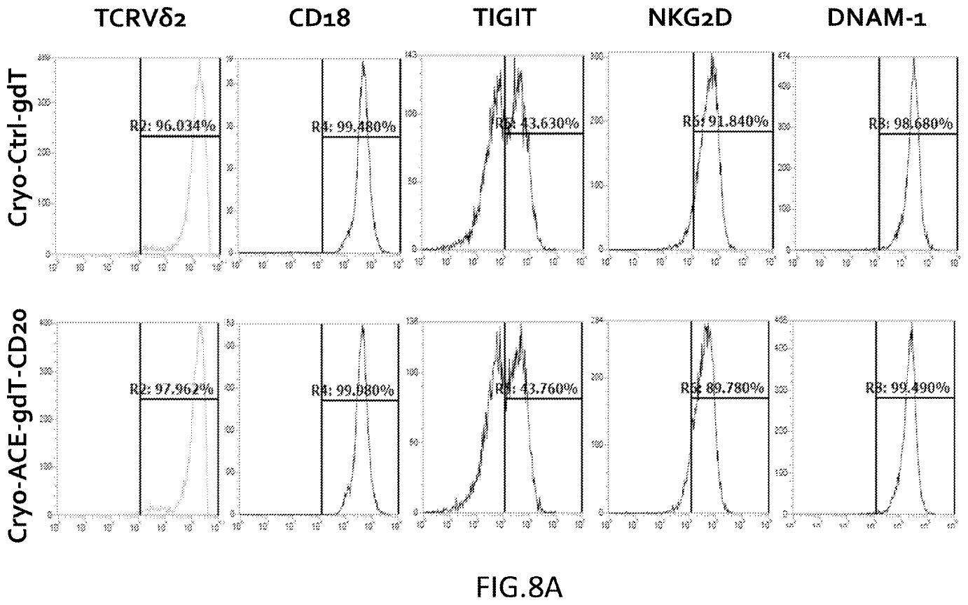

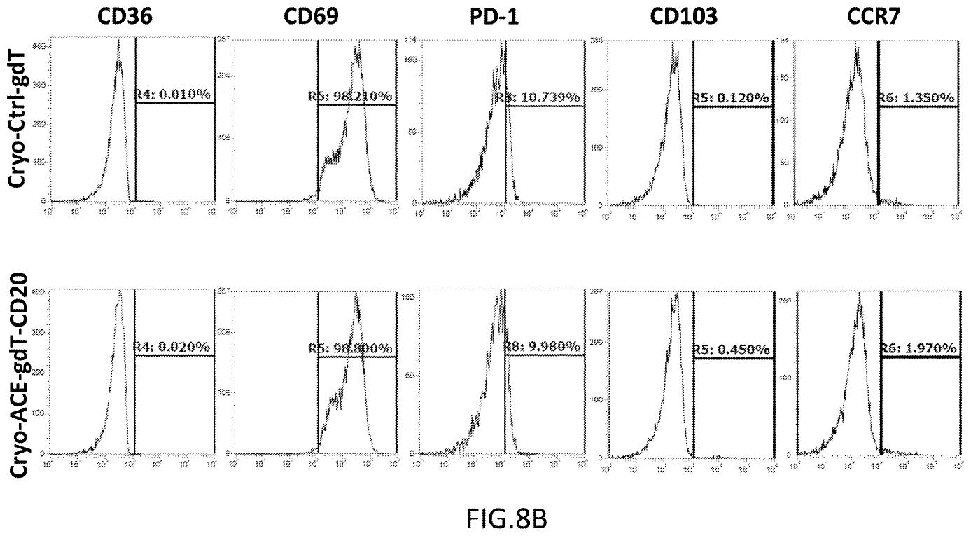

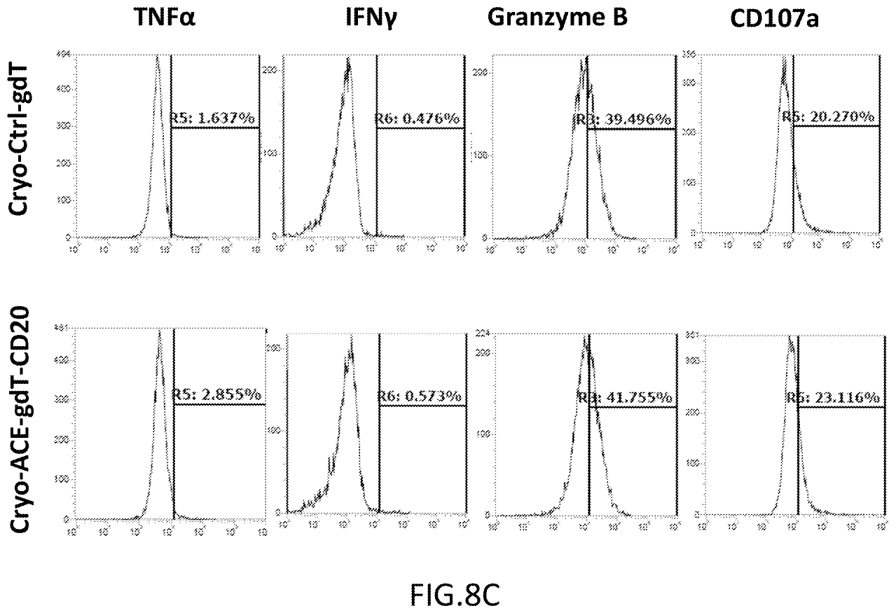

As shown in FIGS. 8A-8C and summarized below, the PI−TCRVβ2+-gated populations in both control group and the Cryo-ACE-gdT-CD20 group were further characterized in percentage/MFI. The percentages were calculated using the number of PI−TCRVβ2+cells (i.e., the gdT cells) as the total number (denominator), and the MFI values were determined in respective gated marker-positive gdT populations.

| | Cryo-Ctrl- | Cryo-ACE- | Cryo-Ctrl- | Cryo-ACE- |
|---|---|---|---|---|
| | gdT | gdT-CD20 | gdT | gdT-CD20 |
| | (%) | (%) | (MFI) | (MFI) |
| CD18+ | 99.48 | 99.98 | 48377 | 47472 |
| TIGIT+ | 43.63 | 43.76 | 5330 | 5232 |
| NKG2D+ | 91.84 | 89.78 | 6193 | 5441 |
| DNAM1+ | 98.68 | 99.49 | 25956 | 24801 |
| CD69+ | 98.21 | 98.80 | 31100 | 27917 |
| PD-1+ | 10.74 | 9.98 | 1729 | 1819 |
| CD103+ | 0.12 | 0.45 | 1991 | 8545 |
| CCR7+ | 1.35 | 1.97 | 4307 | 6839 |
| TNFα+ | 1.64 | 2.86 | 2798 | 2900 |
| IFNγ+ | 0.48 | 0.57 | 3105 | 3638 |
| GZMB+ | 39.50 | 41.76 | 2818 | 2823 |
| CD107a+ | 20.27 | 23.12 | 3487 | 3463 |

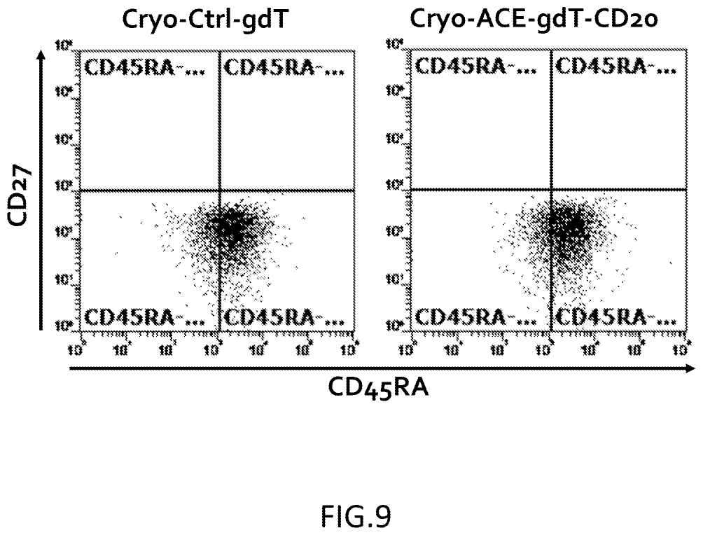

The phenotype of PI−TCRVβ2+-gated populations were also analyzed to distinguish between naïve (CD45RA+CD27+), CM (CD45RA−CD27+), EM (CD45RA−CD27−) and TDEM (CD45RA+CD27−) cells. As shown in FIG. 9, PI−TCRVβ2+-gated cells in the control group were primarily enriched in EM cells (CD45RA−CD27−; 26.43%) and TDEM cells (CD45RA+CD27−; 73.57%) populations. Consistently, the PI−TCRVβ2+-gated cells in the ACE-gdT-CD20 group were also primarily enriched in EM cells (CD45RA−CD27−; 21.47%) and TDEM cells (CD45RA+CD27−; 78.53%).

### 5.4.5 Cytotoxicity of Ctrl-gdT Cells and ACE-gdT-HER2 Cells

xCELLigence Real Time Cell Analysis System (xCELLigence RTCA system, ACEA Biosciences Inc.) was used to measure the cytotoxicity of effector cells toward target cells. 96 well xCELLigence E-Plates were used, and the wells were divided into effector cell alone control wells (ESA), target cell alone control wells (TSA), experimental wells, and target cell total lysis control wells (TML). ACE-gdT-HER2 and Ctrl-gdT cell populations prepared as described above were used as effector cells, and SK-OV-3 cell line (HTB-77, ATCC), an adherent ovarian cancer cell line, was used as the target cells.

2×10⁴ SK-OV-3 cells were seeded in each of the TSA wells, experimental wells, and TML wells, which were allowed to sit 2-4 hours. When cell index (CI) of target cells reaches 0.5, effector cells (ACE-gdT-HER2 or Ctrl-gdT) were added to the ESA wells and experimental wells, to reach an E:T ratio (the ratio of the effector cell number to the target cell number) of 1, 2, 5 or 10. Lysis buffer was added into the TML cells to determine the CI of the wells as all of the target cells in these wells were lysed. No effector cell or lysis buffer was added to the TSA wells.

Control-gdT cells were also seeded in the presence of trastuzumab at different concentrations (10 ng/mL, 100 ng/mL, 1 μg/mL, or 10 μg/mL). For these samples, E:T ratio was 2.

The xCELLigence E-Plates were placed in the xCELLigence Real Time Cell Analysis System for 18 hours to detect real time change in the CI (37° C. and 5% carbon dioxide). The more target cells attached to the bottom of the xCELLigence E-Plate, the higher the detected CI. Therefore, the CI were used to convert the percentage of target cells that were lysed in experimental wells according to the following formula: Percentage of lysed target cell (%)={1−[(CI of experimental well−CI of ESA wells−CI of TML wells)+(CI of TSA wells−CI of TML wells)]}×100%

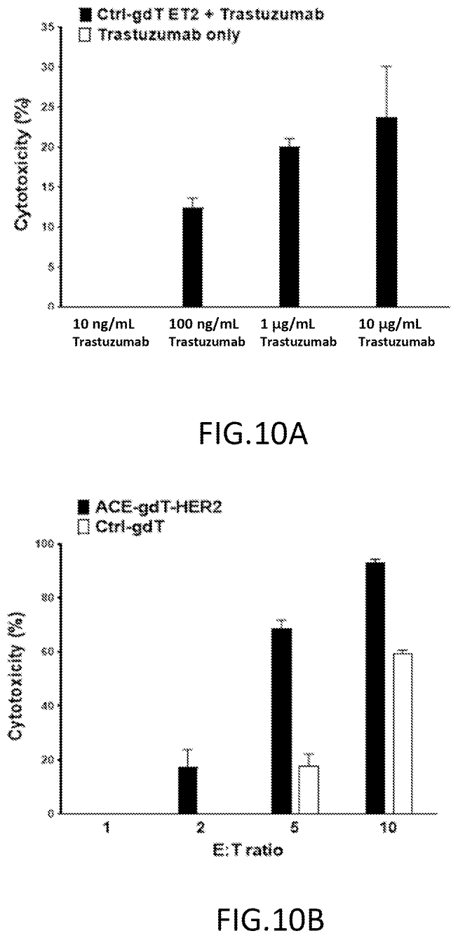

As shown in FIG. 10A, trastuzumab alone did not kill target cell SK-OV-3 at any concentration, whereas in the presence of trastuzumab, up to 22% of the target cell SK-OV-3 were lysed by the control gdT cells. This result demonstrated the cytotoxicity of the cell populations prepared as disclosed herein, which was dose dependent on the presence of antibody, demonstrating that the control gdT cells mediated an ADCC response. As further shown in FIG. 10B, the ACE-gdT-HER2 cells killed 0%, 18%, 68% and 92% of SK-OV-3 at the E/T of 1, 2, 5 and 10, respectively; while control-gdT cells killed 0%, 0%, 15% and 58% of SK-OV-3 at E/T 1, 2, 5 and 10, respectively. This result showed the cytotoxicity of the Ctrl-gdT cells against SK-OV-3, which was further enhanced with trastuzumab conjugation, as observed for the ACE-gdT-HER2 cells.

### 5.4.6 Cytotoxicity of Ctrl-gdT Cells and ACE-gdT-CD20 Cells

CD20+Daudi human lymphoma cell line, CD20+Raji human lymphoma cell line, and CD20-K562 human lymphoma cell line were purchased from ATCC and used as the target cells. The target cells were spun down (400×g, 3 min), resuspended with 1 mL RPMI growth media and adjusted to 2×10⁶ cells/ml. 6 millions of target cells were stained in DPBS with 5 μM fluorescent dye carboxyfluorescein succinimidyl ester (CFSE, ThermoFisher Scientific) for 10 minutes at room temperature according to manufacturer's instruction. The stained cells were washed twice with DPBS and seeded in a 24-well cell culture plate (1 million per well). ACE-gdT-CD20 (rituximab-conjugated gdT cells) and Ctrl-gdT cell populations prepared as described in Section 5.4.3 were used as effector cells.

CFSE-stained target cells (2×10⁵) were co-incubated with effector cells at E:T ratio of 2:1, 5:1 or 10:1 in the 24-well cell culture plate at 37° C. in 5% of CO2 for 4 hours. The cell cultures were harvested and stained with PI at 1:500 dilution. The cytotoxicity was determined by using flow cytometry: percentage of lysed target cell (%)=the number of the PI+cells in 10000 gated CFSE+target cells

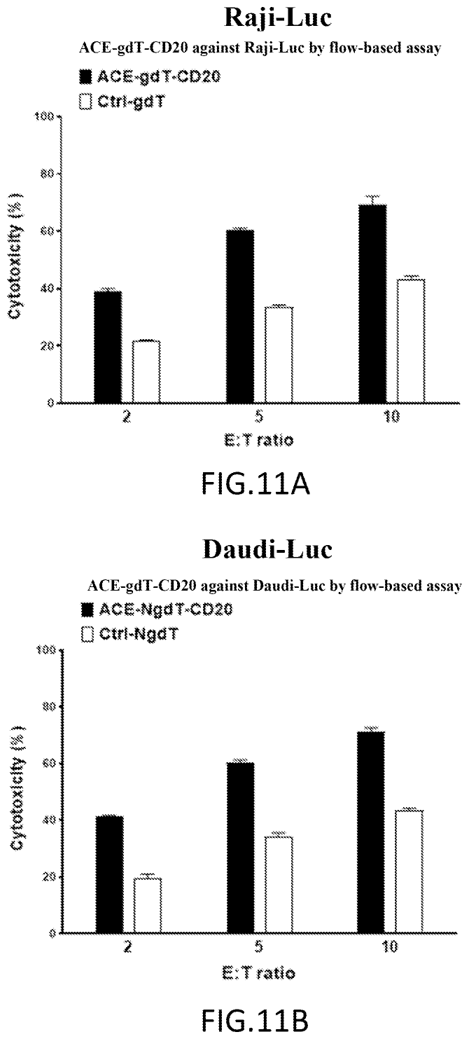

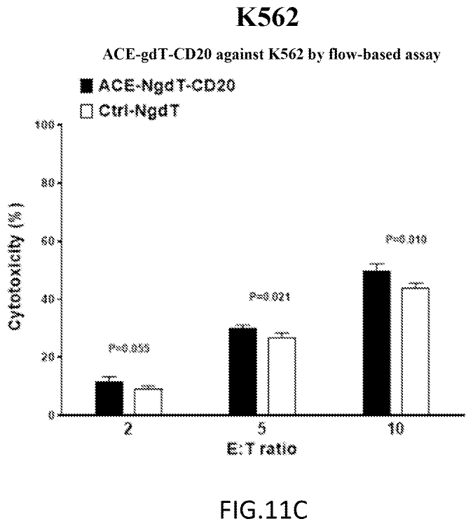

The results (percentage of lysed target cells) are shown in FIGS. 11A-11C. The bar chart in FIG. 11A presenting the comparison of cytotoxic function between the control gdT cells and the rituximab-conjugated gdT cells to kill CD20-positive human lymphoma cell line Raji at different effector (E) to target (T) ratio. In CD20-positive Raji-Luc model, the cytotoxicity exerted by the control gdT cells in the control group (Ctrl-gdT) were 21.95±0.21%-43.13±1.29% from E:T ratio of 2:1 to 10:1, whereas ACE-gdT-CD20 cells killed 39.14±0.86%-69.38±2.77% of the target cells. The bar chart in FIG. 11B presenting the comparison of cytotoxic function between the control gdT cells and the ACE-gdT-CD20 cells to kill CD20-positive human lymphoma cell line Daudi at different effector (E) to target (T) ratio. In CD20-positive Daudi-Luc model, the cytotoxicity exerted by the control gdT cells in the control group (Ctrl-gdT) were 19.68±1.38%-43.66±0.66% from E:T ratio of 2:1 to 10:1, whereas ACE-gdT-CD20 cells killed 41.19±0.6%-71.22±1.42% of the target cells. The bar chart in FIG. 11C presenting the comparison of cytotoxic function between the control gdT cells and the rituximab-conjugated gdT cells to kill CD20-negative human lymphoma cell line K562 at different effector (E) to target (T) ratio. In CD20-negative K562 human lymphoma cell line model, the results demonstrated that the control gdT cells in the control group (Ctrl-gdT) killed 9.31±0.80%-44.12±1.41% of target cells from E:T ratio of 2:1 to 10:1 and ACE-gdT-CD20 cells killed 11.59±1.76%-49.93±2.31% of target cells in the same range of E:T ratio. As provided, Ctrl-gdT cells demonstrated dose-dependent cytotoxicity against all tumor cell lines, whereas the ACE-gdT-CD20 cell population demonstrated enhanced cytotoxicity against CD20+tumor cell lines.

### 5.4.7 Maintenance of Cytotoxicity During Cryopreservation

The study described below was to confirm that cryopreservation would not affect the cytotoxicity of the cell populations disclosed herein. Target cells (Daudi cells and Raji cells) were prepared as described above. The effector cells used in this study included (1) fresh Day 16 resulting cell populations; (2) fresh ACE-gdT-CD20 cell populations prepared as described in Section 5.4.3; (3) cryopreserved and thawed Day 16 resulting cell populations; (4) cryopreserved and thawed ACE-gdT-CD20 cell populations. PBMC cells derived from three difference donors (donors 1, 2, and 3) were used in parallel experiments.

CFSE-stained target cells (2×10⁵) were co-incubated with effector cells at E:T ratio of 2:1, 5:1 and 10:1 in wells of the 24-well cell culture plate at 37° C. in 5% of CO₂ for 4 hours. The cell cultures were harvested and stained with PI at 1:500 dilution. The cytotoxicity was determined by using flow cytometry: Percentage of lysed target cell (%)=the number of the PI+cells in 10000 gated CFSE+target cells.

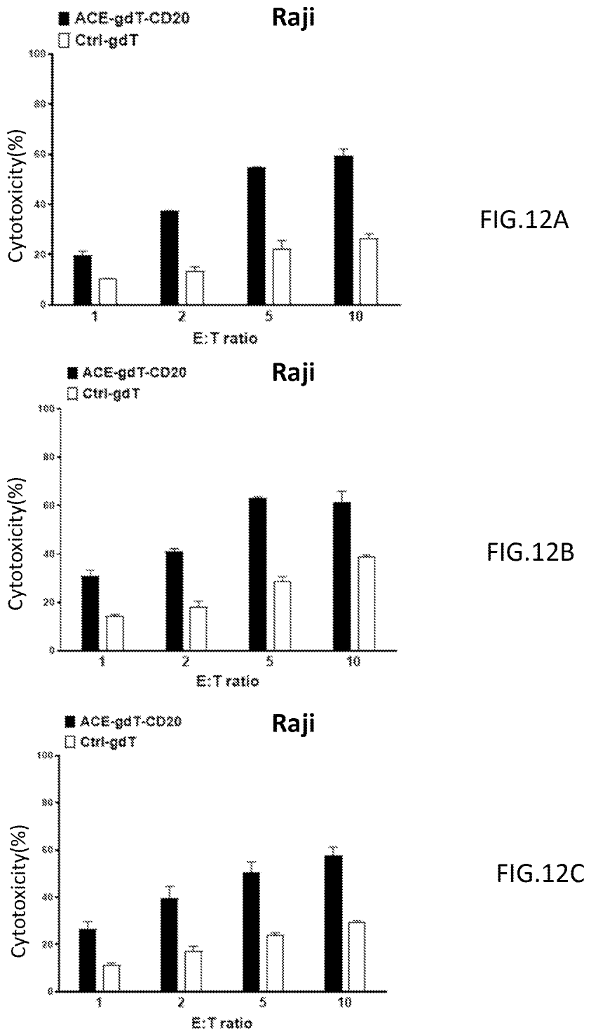

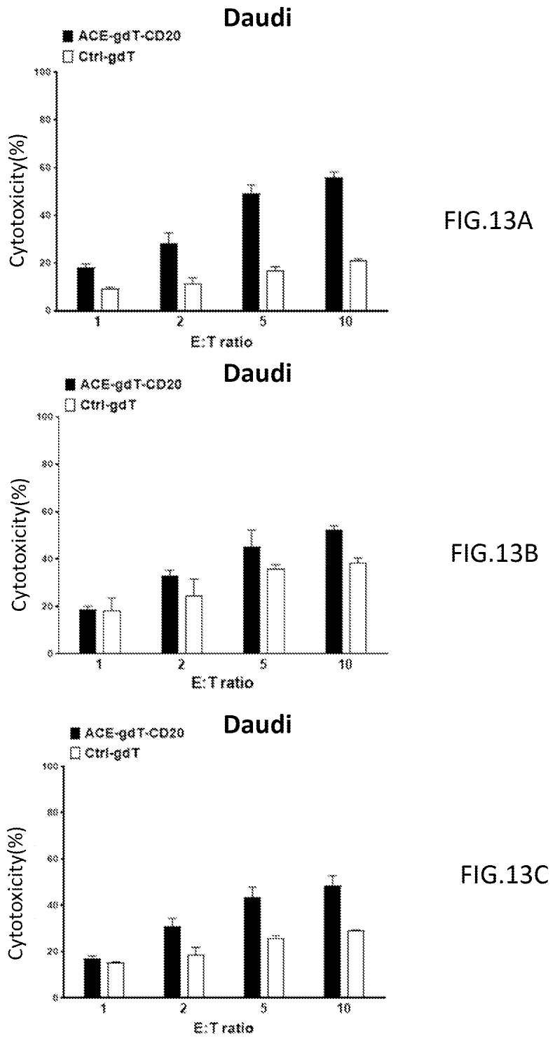

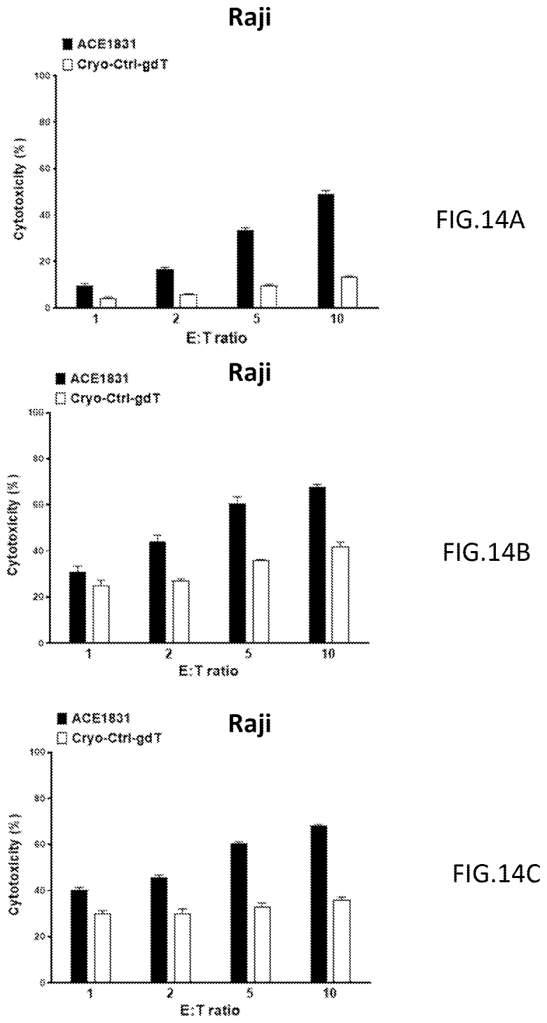

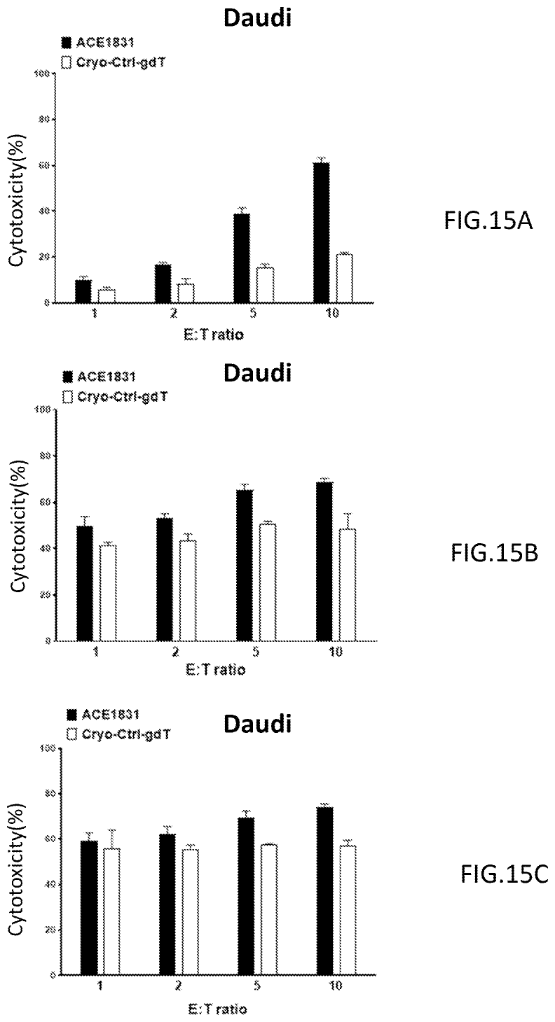

The results are shown in FIGS. 12A-12C (fresh cell populations in Raji model), 13A-13C (fresh cell populations in Daudi model), 14A-14C (cryopreserved cell populations in Raji model), and 15A-15C (cryopreserved cell populations in Daudi model) and summarized below. Panels A, B, and C corresponded to results from Donor 1, 2, and 3, respectively. In Daudi-Luc model, the cytotoxicity exerted by fresh Ctrl-gdT cells (fresh 16-Day cultured PBMC cells) were 9.18±0.62%-38.59±1.93% from E:T ratio of 1:1 to 10:1, whereas fresh ACE-gdT-CD20 cells (fresh CD20-linked 16-Day cultured PBMC cells) killed 16.99±1.16%-55.95±2.21% of the target cells. In Raji-Luc model, cryopreserved Ctrl-gdT cells (cryopreserved 16-Day cultured PBMC cells) were 4.40±0.28%-41.96±1.85% from E:T ratio of 1:1 to 10:1, whereas cryopreserved ACE-gdT-CD20 cells (cryopreserved CD20-linked 16-Day cultured PBMC cells) killed 9.50±1.05%-68.13±0.47% of the target cells. In Daudi-Luc model, the cytotoxicity exerted by cryopreserved Ctrl-gdT cells (cryopreserved 16-Day cultured PBMC cells) were 5.83±0.95%-56.94±2.47% from E:T ratio of 1:1 to 10:1, whereas cryopreserved ACE-gdT-CD20 cells (cryopreserved CD20-linked 16-Day cultured PBMC cells) killed 10.02±1.29%-74.01±1.51% of the target cells.

As provided, the cytotoxicity of the cell populations used in this study was largely maintained after cryopreservation and thawing.

### 5.4.8 Effects of Human Platelet Lysate

The methods for preparing cell populations enriched in gdT cells with NK-like properties as described in Section 5.4.1 were repeated wherein the culture media were supplemented with three different concentrations of HPL (1, 5, or 20 vol % HPL). Glucose levels and cell numbers were monitored during the culture period. Samples were obtained on different days, mixed with an equal volume of Trypan blue, and cell numbers counted. The total cell number in the culture was calculated accordingly.

| | Day | 1 vol % HPL | 5 vol % HPL | 20 vol % HPL |
|---|---|---|---|---|
| Viability (%) - | 0 | 97.7 | 97.7 | 97.7 |
| Batch #2 | 7 | 95.3 | 95.0 | 97.3 |
| | 10 | 85.0 | 97.0 | 85.0 |
| | 14 | 90.7 | 93.7 | 85.3 |
| Total cell | 0 | 3 | 3 | 3 |
| number (10⁷) - | 7* | 3.11 (2.4) | 11.64 (5.78) | 11.13 (5.82) |
| Batch #1 | 10 | 0.24 | 13.23 | 15.23 |
| | 14 | 0.09 | 49.53 | 18.33 |
| Total cell | 0 | 3 | 3 | 3 |
| number (10⁷) - | 7 | 1.82 | 14.29 | 10.79 |
| Batch #2 | 10 | — | 27.86 | 22.18 |
| | 14 | — | 55.11 | 63.70 |
| | 16 | — | >101.2 | 62.40 |

*In Batch #1, a subpopulation of the cells was reseeded on Day 7. The numbers in parentheticals corresponded to the numbers of cells reseeded for further expansion.

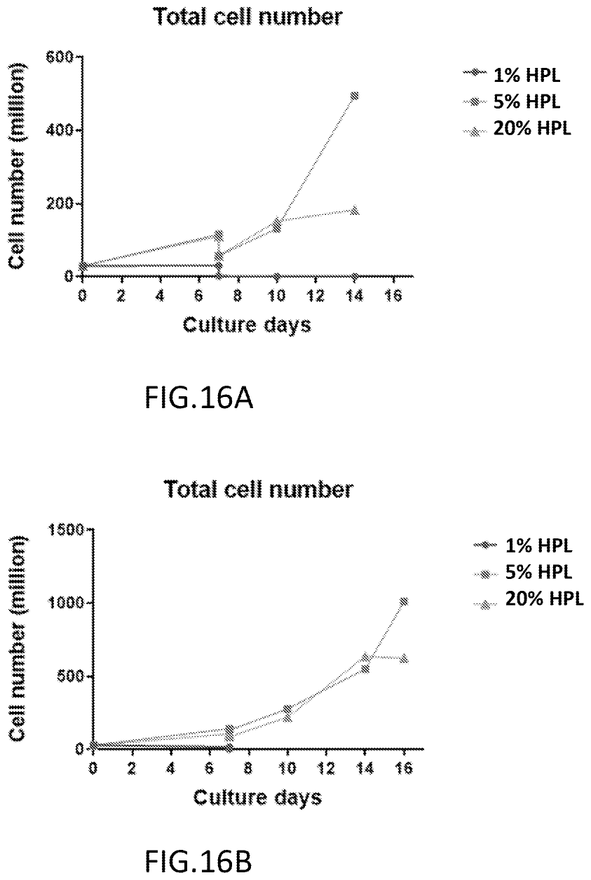

FIGS. 16A-16B provides the cell numbers measured during the culture, which were further summarized in the table above together with the cell viability data. As shown, rapid expansion of gdT cell populations was observed in both the 5 and 20 vol % HPL groups, with the 5 vol % HPL group showing the greatest expansion at the end of the culture.

The marker profiling of the PI−TCRVβ2+-gated cell population was performed according to methods described in Sections 5.4.2 and 5.4.4 above. Note that the percentages were calculated using the number of PI−TCRVβ2+cells (i.e., the gdT cells) as the total number (denominator), but the MFI-based NMC values in the gdT population (instead of the marker-positive subpopulations) were determined for each marker. Results were summarized below. Together, these results demonstrated the NK-like cytotoxic properties of the gdT cells in the resulting cell populations cultured in media comprising either 5% or 20 vol % HPL.

| | | 5 vol % | 20 vol % | 5 vol % | 20 vol % |
|---|---|---|---|---|---|
| | | HPL | HPL | HPL | HPL |
| Day | Marker | % | % | NMC | NMC |
| 16 | CD56+ | 76.410 | 76.779 | 77336 | 75338 |
| | CD16+ | 80.573 | 76.410 | 87202 | 87910 |
| | NKG2D+ | 96.842 | 98.574 | 61721 | 86714 |
| | CD69+ | 40.943 | 79.206 | 35674 | 61442 |
| | DNAM1+ | 88.399 | 88.322 | 220408 | 142280 |

Also, as shown below, the resulting cell populations in both the 5 and 20 vol % HPL groups were predominantly EM cells and TDEM cells, with only about 1% naïve cells and about 1% CM cells, again supporting the therapeutic potential of the resulting cell populations.

| | Naive | CM | EM | TDEM |
|---|---|---|---|---|
| Phenotype | CD45RA+CD27+ | CD45RA−CD27+ | CD45RA−CD27− | CD45RA+CD27− |
| 5 vol % HPL | 0.452 | 0.552 | 54.696 | 44.301 |
| 20 vol % HPL | 0.621 | 1.381 | 74.555 | 23.443 |

The cytotoxicity of the resulting cell populations was also analyzed. Briefly, target Raji cells were seeded in 96-well plates (5,000 per well), and the resulting cell populations in both the 5 and 20 vol % HPL groups were co-incubated with Raji cells at E:T ratio of 2:1, 5:1 and 10:1 for 4 hours. The culture was then analyzed by the luminescence-based cytotoxicity assay described in Sections 5.4.5 and 5.4.6 above.

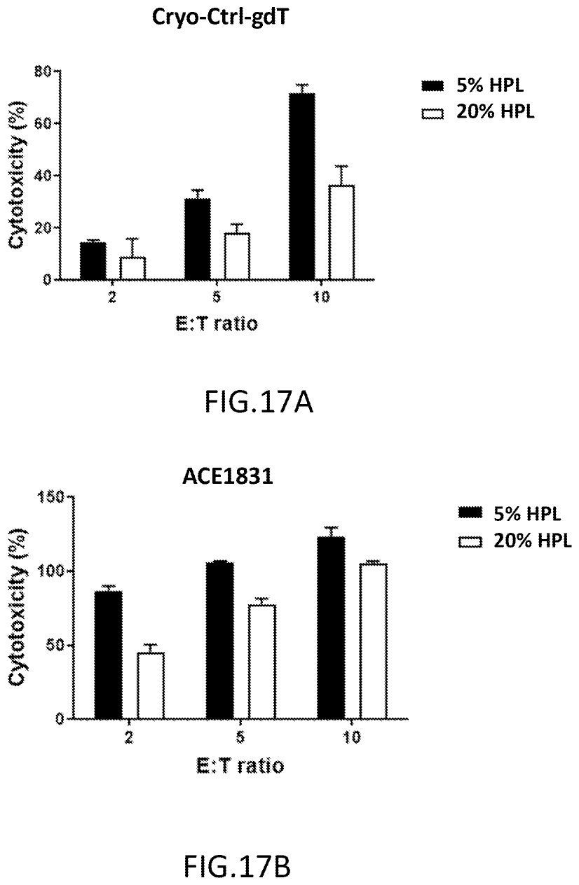

As shown in FIGS. 17A-17B, potent cytotoxicity was observed in both the 5 and 20 vol % HPL groups. Higher cytotoxicity was observed in the 5% HPL group, which could result from the increased expression of DNAM1 on the gdT cells (measured in NMC) and/or the increased percentage of TDEM cells in this group.

5.4.9 Effects of the containers

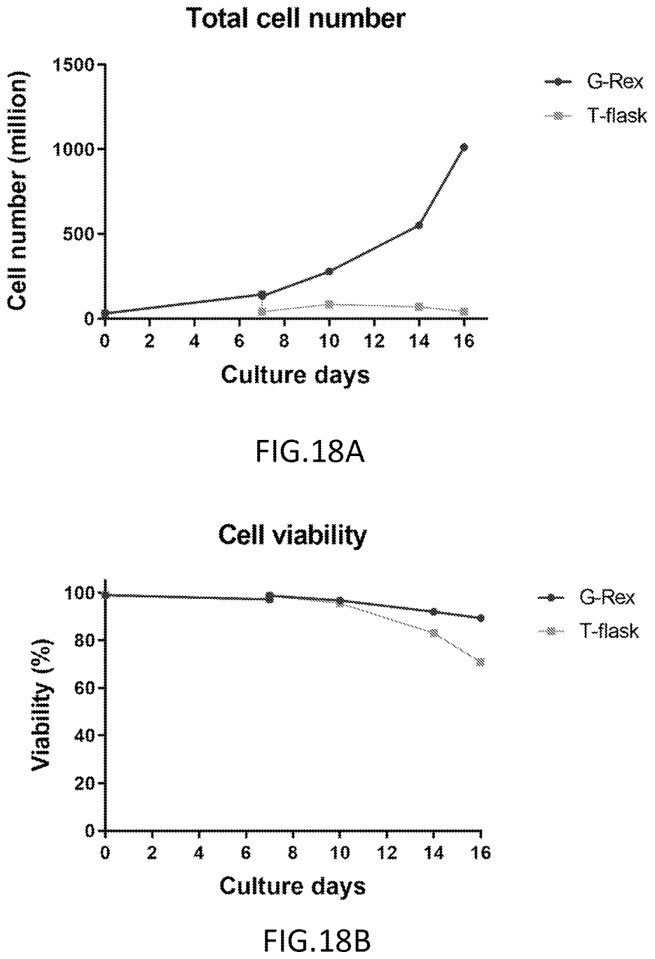

The methods for preparing cell populations enriched in gdT cells with NK-like properties as described in Section 5.4.1 were repeated, except that different cell culture containers were used, including (1) an air-permeable cell culture container (G-Rex device) and (2) an air-impermeable cell culture container (T flask). Glucose levels and cell numbers were monitored during the culture period. Samples were obtained on different days, mixed with an equal volume of Trypan blue, and cell numbers counted. The total cell number in the culture was calculated accordingly. As shown in FIG. 18A, the cell population expanded rapidly between Day 7 and Day 16 when cultured in G-Rex, but not when cultured in air-impermissible T-flask. A decrease in cell viability was also observed when the cell population was cultured in T-flask (FIG. 18B).

The marker profiling was performed according to methods described in Sections 5.4.2 and 5.4.4 above for CD56, CD16, DNAM-1, NKG2D, and CD69 in the PI−TCRVβ2+-gated cell population. Both percentages of marker-positive populations and MFI-based NMC were summarized below. Note that the percentages were calculated using the number of PI−TCRVβ2+cells (i.e., the gdT cells) as the total number (denominator), but the MFI-based NMC values in the gdT population (instead of the mark-positive subpopulations) were determined for each marker. As shown, significantly higher expression of certain activation markers for gdT cells (e.g., CD56, CD16, and DNAM) was observed in the G-Rex group.

| | | G-Rex | T-flask | G-Rex | T-flask |
|---|---|---|---|---|---|
| Day | Marker | % | % | NMC | NMC |
| 16 | CD56+ | 76.410 | 57.483 | 77336 | 61502 |
| | CD16+ | 80.573 | 57.193 | 87202 | 36276 |
| | DNAM1+ | 88.399 | 69.130 | 220408 | 56732 |
| | NKG2D+ | 96.842 | 95.979 | 61721 | 60642 |
| | CD69+ | 40.943 | 50.838 | 35674 | 33499 |

Also, as shown below, the resulting cell populations in both the G-Rex- and T-flask-cultured groups were predominantly EM cells and TDEM cells, with only about 1% naïve cells and about 1% CM cells.

| | Naive | CM | EM | TDEM |
|---|---|---|---|---|
| Phenotype | CD45RA+CD27+ | CD45RA−CD27+ | CD45RA−CD27− | CD45RA+CD27− |
| G-Rex | 0.452 | 0.552 | 54.696 | 44.301 |
| T-flask | 2.233 | 11.736 | 66.954 | 19.077 |

These results demonstrated that cell populations prepared using containers with better air-permeability harbored better expansion capability and higher toxicity as demonstrated by the higher percentages and mean numbers per cell of activation receptors including cytotoxicity receptors (CD56, CD16, DNAM-1) and the percentage of TDEM population. As shown, a significantly higher percentage of TDEM cells was observed in the G-Rex group.

### 5.4.10 Effects of αβT Depletion

The methods for preparing cell populations enriched in gdT cells with NK-like properties as described in Section 5.4.1 were repeated with (1) depletion of abT cells performed on Day 6, (2) depletion of abT cells not performed; (3) depletion of abT cells performed on Day 16 or (4) depletion of abT cells performed on Day 0.

Cell numbers were monitored during the culture period. Samples were obtained on different days, mixed with an equal volume of Trypan blue, and cell numbers counted. The total cell number in the culture was calculated accordingly. On Day 16, Group 1 had 1.55×10¹⁰ cells, Group 2 had 5×10⁹ cells, Group 3 had 2.74×10⁹ cells, and Group 4 failed to expand. Group 2 had 8.52% abT cells before depletion, whereas Group 3 had 20% abT cells before depletion.

These results demonstrated that abT cells could be depleted after a few days (e.g., 1-5 days) of ex vivo culture of the cell population. Additionally, that Group 3 had less cells than Group 2 indicated that depleting the abT cells when their percentage was relatively low (e.g., less than 10%) would help achieve the highest number of gdT cells by the end of the culture.

The marker profiling is also to be performed according to methods described in Sections 5.4.2 and 5.4.4 above.

### 5.4.11 CD69+ as the Marker for Cytotoxicity

To evaluate the receptor CD69 as a positive marker for cytotoxicity of the resulting cell population, the cytotoxicity assay described in sections above can be performed. For example, Raji cells can be used as the target cells, and (1) CD69+gdT cells and (2) CD69− gdT cells isolated from Day 16 resulting cell population prepared as described in Section 5.4.1 can be used as the effector cells.

CD69+cells and CD69− cells can be separated using different methods. For example, TCRVδ2+cells can be isolated from Day 16 resulting cell population, and CD69 magnetic microbeads (Miltenyi) can be used to separate CD69+(microbeads-bound) and CD69− (eluted) gdT cells. For another example, human CD69 MicroBead Kit II (Miltenyi Biotech) can be used and CD69+gdT cells can be isolated according to isolation kit manufacturer's instruction. In brief, about 1×10⁷ harvested gdT cells suspended in the 40 μL of autoMACS running buffer are incubated with 10 μL biotin-conjugated anti-CD69 antibody supplied in the kit at 4° C. for 15 minutes. The binding can be scaled up proportionally. The aforementioned mixture is mixed with about 20 μL of anti-Biotin MicroBeads per 10⁷ cells and 30 μL of autoMACS running buffer, the total 100 uL of mixture is incubated at 4° C. for 15 minutes. The stained cells are washed with 1-2 mL autoMACS running buffer per 10⁷ cells followed by 400×g for 5 minutes. The centrifuged cells are resuspended with autoMACS running buffer at 500 μL per 10⁸ cells and loaded into equilibrated depletion column to separate fraction of CD69+ and CD69− population of gdT cells.

Populations with different proportions of CD69+ and CD69− gdT cells can be prepared by mixing the separated fraction of CD69+ and CD69− populations of gdT cells, for example, with ratio of CD69+ to CD69− at 0:1, 1:2, 1:1, 2:1, and 1:0. The mixed populations can then be subjected to marker profile analysis and cytotoxicity assay as described in sections above.

### 5.4.12 Tumor Killing by Ctrl-gdT Cells and ACE-gdT-CD20 Cells in Mouse Models

Twelve-week-old female SCID-Beige mice (BioLasco Taiwan Co., Ltd.) were intravenously injected with 1×10⁵ CD20-expressing Raji/Luc cells in 100 μL of serum-free medium on Day 0 and separated into three groups: vehicle group, control-gdT group, and ACE-gdT-CD20 group. ACE-gdT-CD20 (rituximab-conjugated gdT) cell populations and Ctrl-gdT cell populations prepared as described in Section 5.4.3 were used as effector cells.

1×10⁷ effector cells (ACE-gdT-CD20 cells or Ctrl-gdT cell) were intravenously injected into mice in the control group and the ACE-gdT-CD20 group, respectively. Mice in the vehicle group were injected with the same volume of serum-free medium. The same treatment was repeated on Day 3, 7 and 10. The bioluminescence of each mouse was monitored by IVIS in vivo imaging system (e.g., PerkinElmer).

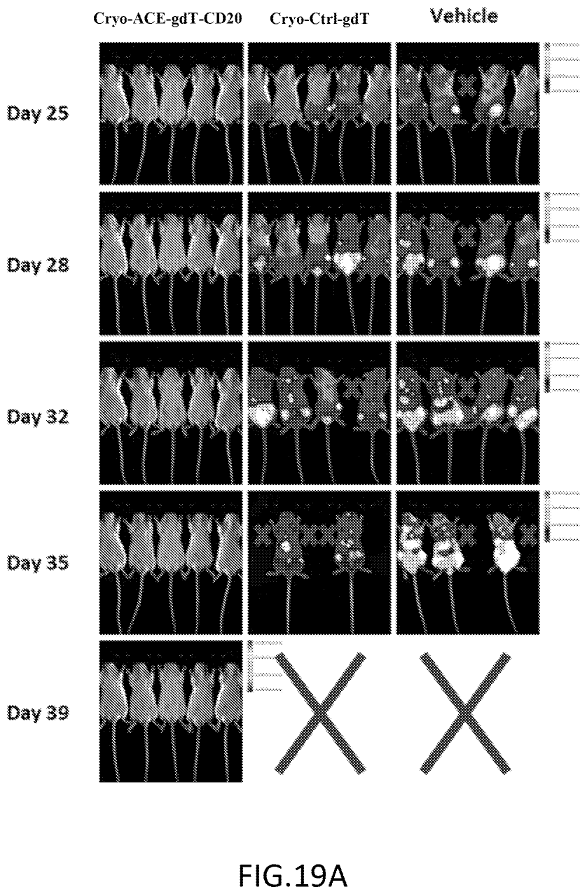

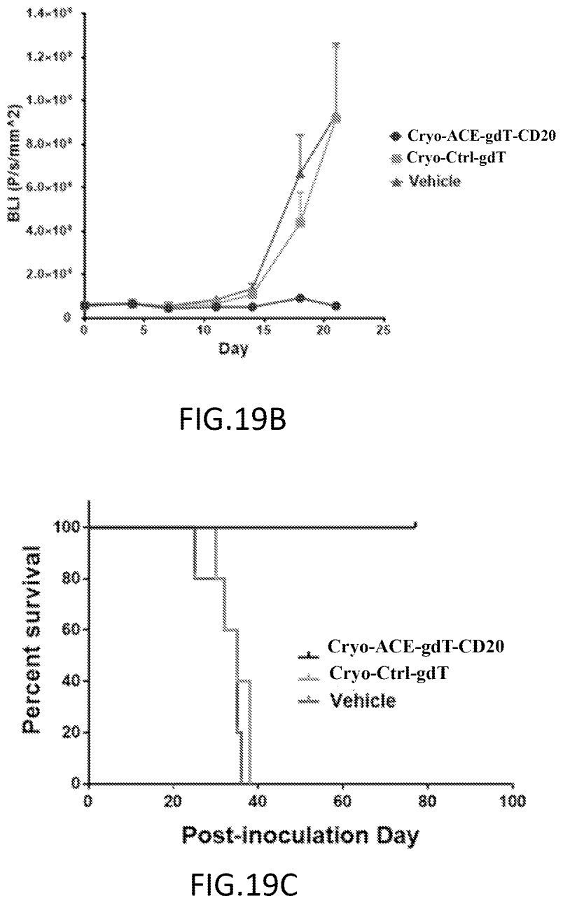

As shown in FIGS. 19A-19C, ACE-gdT-CD20 cell population showed potent anti-tumor activity and suppressed the tumor burden throughout the treatment. Mice administered with ACE-gdT-CD20 showed significantly improved survival compared to the other two groups.

5.4.13 Tumor Killing by CD69+gdT Cells in Mouse Model with Liquid Tumor.

CD69+gdT cells and CD69− gdT cells described in Section 5.4.11 are mixed to prepare compositions having different amounts of CD69+cells. The tumor killing activities of these cell populations are measured in mouse model.

One-time administration: 5×10⁵ luciferase-expressing target cells (Raji) are intravenously injected into each of the 35 female immune compromised NSG mice (Jackson Laboratory) or SCID-Beige mice (BioLasco Taiwan Co., Ltd.) on Day 0. The mice are divided into 7 groups and administered with the different amounts of CD69+Ctrl-gdT cells as the effector cells. Group 1: 2×10⁶; Group 2: 5×10⁶; Group 3: 1×10⁷; Group 4: 2×10⁷; Group 5: 3×10⁷; Group 6: 5×10⁷; Group 7: 0 (medium only)

Luminescence is detected by in vivo imaging system (e.g., AMI HTX (Spectral Imaging); IVIS (PerkimElmer)) on Day 0, 3, 8, 11, 18, 25 and 32. The bioluminescence images of mice are expected to show dose-dependent tumor reduction in mice administered with CD69+Ctrl-gdT cells.

Multiple administrations: Same target cells are used as described above; various amounts of effector cells (CD69+Ctrl-gdT) are used following the treatment plan below.

| | cells per | Number of | Total injected |
|---|---|---|---|
| Group | injection | injections | cells |
| 1 | 7.5 × 10⁶ | 2 (Days 0 and 3) | 1.5 × 10⁷ |
| 2 | 7.5 × 10⁶ | 4 (Days 0, 3, 7, and 10) | 3 × 10⁷ |
| 3 | 7.5 × 10⁶ | 8 (Days 0, 3, 7, 10, 14, | 6 × 10⁷ |
| | | 17, 21 and 24) | |
| 4 | 1.5 × 10⁷ | 1 (Day 0) | 1.5 × 10⁷ |
| 5 | 3 × 10⁷ | 1 (Day 0) | 3 × 10⁷ |
| 6 | 6 × 10⁷ | 1 (Day 0) | 6 × 10⁷ |
| 7 | 0 (medium only) | 8 (Days 0, 3, 7, 10, 14, | 0 |
| | | 17, 21 and 24) | |

Luminescence is detected by in vivo imaging (e.g., AMI HTX (Spectral Imaging); IVIS (PerkimElmer)) on Days 0, 3, 8, 11, 15, 18, 22, 25, 32, 39, 46, 53 and 60. The bioluminescence images of mice are expected to show dose-dependent tumor reduction in mice administered with CD69+Ctrl-gdT cells, and potent anti-tumor activities are expected if a total of at least 1.5×10⁷ CD69+Ctrl-gdT cells are injected. When the unit dose is low (e.g., 7.5×10⁶ in Groups 1-3), it is expected that multiple injections are needed to achieve significant therapeutic effects. When the unit dose is high (e.g., 6×10⁷ in Group 6), potent anti-tumor activities are expected even with only one injection. Low number of injections can help with patient compliance.

5.4.14 Tumor Killing by CD69+gdT Cells in Mouse Model with Solid Tumor

The studies described in Section 5.4.13 (both single administration and multiple administrations) are repeated using SK-OV-3 cells (CELL BIOLABS Inc) as the target cells.

The bioluminescence images of mice are expected to show dose-dependent tumor reduction in mice administered with CD69+Ctrl-gdT cells, and potent anti-tumor activities are expected if a total of at least 1.5×10⁷ CD69+Ctrl-gdT cells are injected. When the unit dose is low (e.g., 7.5×10⁶ in Groups 1-3), it is expected that multiple injections are needed to achieve significant therapeutic effects. When the unit dose is high (e.g., 6×10⁷ in Group 6), potent anti-tumor activities are expected even with only one injection. Low number of injections can help with patient compliance.

5.4.15 Prepare gdT Cells with NK-Like Properties with Different Supplements

The methods for preparing cell populations enriched in gdT cells with NK-like properties as described in Section 5.4.1 are repeated wherein the 5 vol % HPL in culture media is replaced with (1) HPL, (2) human AB serum, or (3) fetal calf serum (FCS) at different concentrations (1 vol %, 5 vol %, or 20 vol % for each supplement) in parallel. Cell numbers are monitored during the culture period and the marker profile analyses and the cytotoxicity assays described in the sections above are performed. Greater expansion of gdT cells and molecule profiles showing the greater NK-cytotoxicity are expected to be observed in the HPL groups.

The foregoing descriptions are merely the preferred embodiments of the present invention and are not intended to limit the scope of the patent application of the present invention. Therefore, any alteration or modification that does not depart from the spirits disclosed herein should be included within the scope of the patent application of the present invention.

All publications mentioned herein are hereby incorporated by reference in their entirety for the purpose of describing and disclosing, for example, the compositions and methodologies that are disclosed herein, which might be used in connection with the presently described inventions. The publications discussed herein are provided solely for their disclosure prior to the filing date of the present application. Nothing herein is to be construed as an admission that the inventors described herein are not entitled to antedate such disclosure by virtue of prior invention or for any other reason.

### 6. REFERENCE TO SEQUENCE LISTING SUBMITTED ELECTRONICALLY

This application incorporates by reference a Sequence Listing with this application as an ASCII text file entitled “GDT_ST25.TXT” created on Apr. 9, 2022 and having a size of 33,393 bytes.

1. A method of manufacturing a cell population enriched in gamma delta T (gdT) cells, comprising culturing a source cell population comprising gdT cells in a medium supplemented with (i) a phosphoantigen, (ii) a cytokine, and (iii) human platelet lysate (“HPL”).

2. The method of claim 1, wherein the cell population is not contacted with a feeder cell or tumor cell during the culture.

3. The method of claim 1 that does not include positively selecting for gdT cells.

4. The method of claim 1, wherein the cell population is cultured for 3 to 40 days, 4 to 40 days, 5 to 40 days, 6 to 40 days, 7 to 40 days, 10 to 40 days, 10 to 30 days, 6 to 20 days, 12 to 20 days, or 14 to 18 days.

5. The method of claim 1, further comprising depleting alpha beta T (abT) cells.

6. The method of claim 5, wherein the abT cells are depleted around the half-time of the culture.

7. The method of claim 5, wherein the cells are cultured for 14 to 18 days and the abT cells are depleted between Day 4 and Day 10.

8. The method of claim 1, wherein the cytokine is replenished during the culture.

9. The method of claim 8, wherein the cytokine is replenished once per week, twice per week, three times per week, every other day, or daily.

10. The method of claim 1, wherein the cytokine is interleukin-2 (IL-2), interleukin-4 (IL-4), interleukin-6 (IL-6), interleukin-7 (IL-7), interleukin-8 (IL-8), interleukin-9 (IL-9), interleukin-12 (IL-12), interleukin-15 (IL-15), interleukin-18 (IL-18), interleukin-21 (IL-21), interleukin-33 (IL-33), or any combination thereof.

11. The method of claim 10, wherein the cytokine is IL-2.

12. The method of claim 1, wherein the cytokine is supplemented at a concentration of 200-3000 IU/mL.

13. The method of claim 1, wherein the phosphoantigen is not replenished during the culture.

14. The method of claim 1, wherein the phosphoantigen is a bisphosphonate selected from the group consisting of clodronate, etidronate, alendronate, pamidronate, zoledronate (zoledronic acid), neridronate, ibandronate, and pamidronate.

15. The method of claim 14, wherein the phosphoantigen is zoledronate.

16. The method of claim 1, wherein the phosphoantigen is selected from the group consisting of bromohydrin pyrophosphate (BrHPP), 4-hydroxy-but-2-enyl pyrophosphate (HMBPP), isopentenyl pyrophosphate (IPP), and dimethylallyl pyrophosphate (DMAPP).

17. The method of claim 1, wherein the phosphoantigen is supplemented at a concentration of 0.1-20 μM.

18. The method of claim 1, wherein the HPL is supplemented at a concentration of 1-20 vol %.

19. The method of claim 1, wherein the medium comprises glucose at a concentration of 600-5000 mg/L.

20. The method claim 1, wherein the medium is a serum-free medium.

21. The method of claim 1, wherein the cell population is cultured in a device containing an air-permeable surface.

22. The method of claim 21, wherein the device is a G-Rex device.

23. The method of claim 1, wherein the source cell population comprises peripheral blood mononuclear cells (PBMCs), bone marrow, umbilical cord blood, or a combination thereof.

24. The method of claim 23, wherein the source cell population comprises PBMCs.

25. The method of claim 24, further comprising obtaining the PBMCs from peripheral blood.

26. The method of claim 1, wherein the gdT cells in the source cell population are expanded for at least 1,000 fold during the culture.

27. The method of claim 1, wherein at least 75% of the resulting cell population are gdT cells.

28. The method of claim 1, further comprising adding a targeting moiety to the surface of the cells in the resulting cell population.

29. The method of claim 28, wherein the targeting moiety is complexed to the cell surface via the interaction between a first linker conjugated to the targeting moiety and a second linker conjugated to the cell surface.

30. The method of claim 28, wherein the targeting moiety is exogenously expressed.

31. The method of claim 1, further comprising cryopreserving the cell population after the culture.

32. A population of cells obtained by the method of claim 1.

33. A population of cells comprising at least 70% gdT cells, wherein (1) the gdT cells express at least 400 DNAM-1 molecules per cell on average; (2) at least 30% of the gdT cells are CD69⁺; or both (1) and (2).

34. The population of cells of claim 33, wherein the gdT cells express at least 500, at least 1000, at least 2000, or at least 3000 DNAM-1 molecules per cell on average.

35. The cell population of claim 33, wherein at least 40%, at least 50%, at least 55%, at least 60%, at least 65%, at least 70%, at least 75%, or at least 80% of the gdT cells are CD69+.

36. The cell population of claim 33, wherein at least 30%, at least 40%, at least 50%, at least 60%, at least 70%, or at least 80% of the gdT cells are terminally differentiated effector (TDEM) cells.

37. The cell population of claim 33, comprising at least 1×10⁶, at least 5×10⁶, at least 1×10⁷, at least 5×10⁷, at least 1×10⁸, at least 5×10⁸, at least 1×10⁹, at least 5×10⁹, at least 1×10¹⁰, at least 5×10¹⁰, or at least 1×10¹¹ gdT cells.

38. The cell population of claim 33, wherein the cell population has not been positively selected for gdT cells.

39. The cell population of claim 33, where the cell population has been cultured for 20 days or less since the source cell population from which the cell population is derived or obtained from a single donor.

40. The cell population of claim 33, wherein:
(1) the gdT cells express at least 400 CD56 molecules per cell on average;
(2) the gdT cells express at least 400 CD16 molecules per cell on average;
(3) the gdT cells express at least 400 NKG2D molecules per cell on average;
(4) the gdT cells express at least 400 CD107a molecules per cell on average;
(5) the gdT cells express at most 2800 PD-1 molecules per cell on average;
(6) the gdT cells express at least 5000 DNAM-1 molecules per cell on average;
(7) the gdT cells express at least 400 CD69 molecules per cell on average; or
(8) the gdT cells express at least 100 Granzyme B molecules per cell on average;
or any combination thereof.

41. The cell population of claim 33, wherein at least 30% of the gdT cells are Vδ2 T cells.

42. The cell population of claim 33, wherein at least 10% of the gdT cells comprise a targeting moiety complexed to the cell surface.

43. The cell population of claim 42, wherein the targeting moiety is not a nucleic acid.

44. The cell population of claim 42, wherein the targeting moiety is an antibody or antigen binding unit that specifically binds to a biological marker on a target cell.

45. The cell population of claim 44, wherein the biological marker is a tumor antigen.

46. The cell population of claim 44, wherein the gdT cells express a chimeric antigen receptor (CAR) or a T cell receptor (TCR) that comprises the antibody or antigen binding fragment.

47. The cell population of claim 42, wherein the targeting moiety is not produced by the gdT cells.

48. The cell population of claim 42, wherein the targeting moiety is complexed to the cell surface via the interaction between a first linker conjugated to the targeting moiety and a second linker conjugated to the cell surface.

49. The cell population of claim 48, wherein the first linker is a first polynucleotide, and the second linker is a second polynucleotide.

50. The cell population of claim 49, wherein (1) the first polynucleotide has 4 to 500 nucleotides, (2) the second polynucleotide has 4 to 500 nucleotides, or both (1) and (2).

51. The cell population of claim 32 that is cryopreserved.

52. A pharmaceutical composition comprising the cell population of claim 32 and a pharmaceutically acceptable carrier.

53. The cell population of claim 32, that can maintain its therapeutic potency after being stored at or below 0° C. for at least one week, at least two weeks, at least 1 month, at least 3 months, or at least 6 months.

54. Use of the cell population or the pharmaceutical composition of claim 32 in an adoptive immunotherapy.

55. Use of the cell population or the pharmaceutical composition of claim 32 in the treatment of a disease or disorder.

56. A method of treating a disease or disorder in a subject in need thereof, comprising administering the cell population or the pharmaceutical composition of claim 32 to the subject.

57. The use of claim 55, wherein the disease or disorder is tumor or cancer.

58. The use of claim 55, wherein the disease or disorder is an autoimmune disease, a neuronal disease, a hematopoietic cell-related disease, metabolic syndrome, a pathogenic disease, HIV or other viral infection, fungal infection, protozoan infection, or bacterial infection.

59. The method of claim 56, wherein the subject is human.


SEQUENCE LISTING (40 sequences)

SEQ ID NO: 1 - DNA, 20 bp, artificial sequence
  linker
cacacacacacacacacaca

SEQ ID NO: 2 - DNA, 20 bp, artificial sequence
  linker
tcatacgactcactctaggg

SEQ ID NO: 3 - DNA, 23 bp, artificial sequence
  linker
agttaccatgacgtcaatttcag

SEQ ID NO: 4 - DNA, 20 bp, artificial sequence
  linker
tgtgtgtgtgtgtgtgtgtg

SEQ ID NO: 5 - DNA, 20 bp, artificial sequence
  linker
ccctagagtgagtcgtatga

SEQ ID NO: 6 - DNA, 23 bp, artificial sequence
  linker
ctgaaattgacgtcatggtaact

SEQ ID NO: 7 - DNA, 20 bp, artificial sequence
  linker
aaaaaaaaaaaaaaaaaaaa

SEQ ID NO: 8 - DNA, 20 bp, artificial sequence
  linker
tttttttttttttttttttt

SEQ ID NO: 9 - DNA, 20 bp, artificial sequence
  linker
actgactgactgactgactg

SEQ ID NO: 10 - DNA, 20 bp, artificial sequence
  linker
cagtcagtcagtcagtcagt

SEQ ID NO: 11 - DNA, 20 bp, artificial sequence
  linker
gtaacgatccagctgtcact

SEQ ID NO: 12 - DNA, 20 bp, artificial sequence
  linker
agtgacagctggatcgttac

SEQ ID NO: 13 - DNA, 20 bp, artificial sequence
  linker
actgatggtaatctgcacct

SEQ ID NO: 14 - DNA, 20 bp, artificial sequence
  linker
aggtgcagattaccatcagt

SEQ ID NO: 15 - DNA, 40 bp, artificial sequence
  linker
aaaaaaaaaaaaaaaaaaaaaaaaaaaaaaaaaaaaaaaa

SEQ ID NO: 16 - DNA, 40 bp, artificial sequence
  linker
tttttttttttttttttttttttttttttttttttttttt

SEQ ID NO: 17 - DNA, 40 bp, artificial sequence
  linker
actgactgactgactgactgactgactgactgactgactg

SEQ ID NO: 18 - DNA, 40 bp, artificial sequence
  linker
cagtcagtcagtcagtcagtcagtcagtcagtcagtcagt

SEQ ID NO: 19 - DNA, 40 bp, artificial sequence
  linker
tgtgtgtgtgtgtgtgtgtgtgtgtgtgtgtgtgtgtgtg

SEQ ID NO: 20 - DNA, 40 bp, artificial sequence
  linker
cacacacacacacacacacacacacacacacacacacaca

SEQ ID NO: 21 - DNA, 40 bp, artificial sequence
  linker
gtaacgatccagctgtcactgtaacgatccagctgtcact

SEQ ID NO: 22 - DNA, 40 bp, artificial sequence
  linker
agtgacagctggatcgttacagtgacagctggatcgttac

SEQ ID NO: 23 - DNA, 40 bp, artificial sequence
  linker
tcatacgactcactctagggtcatacgactcactctaggg

SEQ ID NO: 24 - DNA, 40 bp, artificial sequence
  linker
ccctagagtgagtcgtatgaccctagagtgagtcgtatga

SEQ ID NO: 25 - DNA, 40 bp, artificial sequence
  linker
actgatggtaatctgcacctactgatggtaatctgcacct

SEQ ID NO: 26 - DNA, 40 bp, artificial sequence
  linker
aggtgcagattaccatcagtaggtgcagattaccatcagt

SEQ ID NO: 27 - protein, 164 aa, artificial sequence
  human CD3 zeta
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 28 - protein, 220 aa, artificial sequence
  human CD28
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 29 - protein, 255 aa, artificial sequence
  human 4-1BB
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 30 - protein, 277 aa, artificial sequence
  human OX40
MCVGARRLGRGPCAALLLLGLGLSTVTGLHCVGDTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRAVAAILGLGLVLGLLGPLAILLALYLLRRDQRLPPDAHKPPGGGSFRTPIQEEQADAHSTLAKI

SEQ ID NO: 31 - protein, 199 aa, artificial sequence
  human ICOS
MKSGLWYFFLFCLRIKVLTGEINGSANYEMFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYESQLCCQLKFWLPIGCAAFVVVCILGCILICWLTKKKYSSSVHDPNGEYMFMRAVNTAKKSRLTDVTL

SEQ ID NO: 32 - protein, 93 aa, artificial sequence
  human DAP10
MIHLGHILFLLLLPVAAAQTTPGERSSLPAFYPGTSGSCSGCGSLSLPLLAGLVAADAVASLLIVGAVFLCARPRRSPAQEDGKVYINMPGRG

SEQ ID NO: 33 - protein, 260 aa, artificial sequence
  human CD27
MARPHPWWLCVLGTLVGLSATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDPLPNPSLTARSSQALSPHPQPTHLPYVSEMLEARTAGHMQTLADFRQLPARTLSTHWPPQRSLCSSDFIRILVIFSGMFLVFTLAGALFLHQRRKYRSNKGESPVEPAEPCHYSCPREEEGSTIPIQEDYRKPEPACSP

SEQ ID NO: 34 - protein, 595 aa, artificial sequence
  human CD30
MRVLLAALGLLFLGALRAFPQDRPFEDTCHGNPSHYYDKAVRRCCYRCPMGLFPTQQCPQRPTDCRKQCEPDYYLDEADRCTACVTCSRDDLVEKTPCAWNSSRVCECRPGMFCSTSAVNSCARCFFHSVCPAGMIVKFPGTAQKNTVCEPASPGVSPACASPENCKEPSSGTIPQAKPTPVSPATSSASTMPVRGGTRLAQEAASKLTRAPDSPSSVGRPSSDPGLSPTQPCPEGSGDCRKQCEPDYYLDEAGRCTACVSCSRDDLVEKTPCAWNSSRTCECRPGMICATSATNSCARCVPYPICAAETVTKPQDMAEKDTTFEAPPLGTQPDCNPTPENGEAPASTSPTQSLLVDSQASKTLPIPTSAPVALSSTGKPVLDAGPVLFWVILVLVVVVGSSAFLLCHRRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 35 - protein, 277 aa, artificial sequence
  human CD40
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRALVVIPIIFGILFAILLVLVFIKKVAKKPTNKAPHPKQEPQEINFPDDLPGSNTAAPVQETLHGCQPVTQEDGKESRISVQERQ

SEQ ID NO: 36 - protein, 21 aa, artificial sequence
  signal peptide of human CD8
MALPVTALLLPLALLLHAARP

SEQ ID NO: 37 - protein, 15 aa, artificial sequence
  linker
GGGGSGGGGSGGGGS

SEQ ID NO: 38 - protein, 235 aa, artificial sequence
  human CD8
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 39 - protein, 458 aa, artificial sequence
  human CD4
MNRGVPFRHLLLVLQLALLPAATQGKKVVLGKKGDTVELTCTASQKKSIQFHWKNSNQIKILGNQGSFLTKGPSKLNDRADSRRSLWDQGNFPLIIKNLKIEDSDTYICEVEDQKEEVQLLVFGLTANSDTHLLQGQSLTLTLESPPGSSPSVQCRSPRGKNIQGGKTLSVSQLELQDSGTWTCTVLQNQKKVEFKIDIVVLAFQKASSIVYKKEGEQVEFSFPLAFTVEKLTGSGELWWQAERASSSKSWITFDLKNKEVSVKRVTQDPKLQMGKKLPLHLTLPQALPQYAGSGNLTLALEAKTGKLHQEVNLVVMRATQLQKNLTCEVWGPTSPKLMLSLKLENKEAKVSKREKAVWVLNPEAGMWQCLLSDSGQVLLESNIKVLPTWSTPVQPMALIVLGGVAGLLLFIGLGIFFCVRCRHRRRQAERMSQIKRLLSEKKTCQCPHRFQKTCSPI

SEQ ID NO: 40 - protein, 86 aa, artificial sequence
  human Fc receptor gamma chain
MIPAVVLLLLLLVEQAAALGEPQLCYILDAILFLYGIVLTLLYCRLKIQVRKAAITSYEKSDGVYTGLSTRNQETYETLKHEKPPQ